(19) United States
(12) Patent Application Publication
(10) Pub. No.: US 2018/0179210 A1
(43) Pub. Date: Jun. 28, 2018

(54) Inhibitors of the TEC Kinase Enzyme Family

(71) Applicant: Pharmascience Inc., Montréal (CA)

(72) Inventors: Alain Laurent, Montréal (CA); Yannick Rose, Montréal (CA); Stephen J. Morris, Montréal (CA)

(73) Assignee: Pharmascience Inc., Montréal, QC (CA)

(21) Appl. No.: 15/577,136

(22) PCT Filed: May 27, 2016
(86) PCT No.: PCT/CA2016/050606

§ 371 Date: Nov. 27, 2017

(30) Foreign Application Priority Data
May 27, 2015 (CA) 2.892.548
Sep. 29, 2015 (CA) 2.906.760

(51) Int. Cl. C07D 487/04; A61K 31/519; A61K 45/06; A61K 47/54; C07D 401/14; A61K 31/506; C07D 403/14; C07D 403/12; C07D 473/34; A61K 31/522

(52) U.S. Cl. CPC C07D 487/04; A61K 31/519; A61K 45/06; A61K 47/545; A61K 31/522; A61K 31/506; C07D 403/14; C07D 403/12; C07D 473/34; C07D 401/14

(57) ABSTRACT

The present invention relates to a novel family of kinases inhibitors. Compounds of this class have been found to have inhibitory activity against members of the TEC kinase family, particularly BTK. The present invention is directed to a compound of Formula I or pharmaceutically acceptable salt, solvate, solvate of salt, stereoisomer, tautomer, isotope, prodrug, complex or biologically active metabolite thereof, for use in therapy.

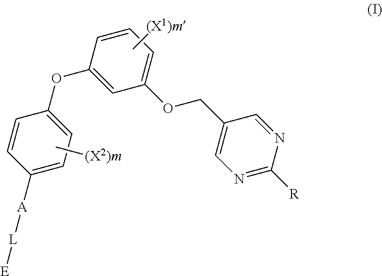

### FIELD OF INVENTION

The present invention relates to a novel family of protein kinase inhibitors, pharmacological compositions that contain them and uses of the inhibitors to treat or prevent diseases, disorders and conditions associated with kinase function.

### BACKGROUND OF THE INVENTION

Protein kinases are a large group of intracellular and transmembrane signaling proteins in eukaryotic cells (Manning G. et al, (2002) Science, 298: 1912-1934). These enzymes are responsible for transfer of the terminal (gamma) phosphate from ATP to specific amino acid residues of target proteins. Phosphorylation of specific amino acid residues in target proteins can modulate their activity leading to profound changes in cellular signaling and metabolism. Protein kinases can be found in the cell membrane, cytosol and organelles such as the nucleus and are responsible for mediating multiple cellular functions including metabolism, cellular growth and differentiation, cellular signaling, modulation of immune responses, and cell death. Serine kinases specifically phosphorylate serine or threonine residues in target proteins. Similarly, tyrosine kinases, including tyrosine receptor kinases, phosphorylate tyrosine residues in target proteins. Tyrosine kinase families include: TEC, Src, Abl, Jak, Csk, Fak, Syk, Fer, Ack and the receptor tyrosine kinase subfamilies including ErbB, FGFR, VEGFR, RET and Eph. Subclass I of the receptor tyrosine kinase superfamily consists of the ErbB receptors and comprises four members: ErbB1 (also called epidermal growth factor receptor (EGFR)), ErbB2, ErbB3 and ErbB4.

Kinases exert control on key biological processes related to health and disease. Furthermore, aberrant activation or excessive expression of various protein kinases are implicated in the mechanism of multiple diseases and disorders characterized by aberrant cellular responses, benign and malignant proliferation, as well as diseases resulting from inappropriate activation of the immune system (Kyttaris V C, Drug Des Devel Ther, 2012, 6:245-50 and Fabbro D. et al. Methods Mol Biol, 2012, 795:1-34). Thus, inhibitors of select kinases or kinase families are expected to be useful in the treatment of cancer, vascular disease, pain, neurological diseases, autoimmune diseases, and inflammatory conditions including, but not limited to: solid tumors, hematological malignancies, thrombus, stroke, Alzheimer's disease, arthritis, Sjogren's syndrome, graft versus host disease, lupus erythematosus, psoriasis, colitis, illeitis, multiple sclerosis, uveitis, coronary artery vasculopathy, systemic sclerosis, atherosclerosis, asthma, transplant rejection, allergy, dermatomyositis, pemphigus, and the like.

Tec kinases are a family of non-receptor tyrosine kinases predominantly, but not exclusively, expressed in cells of hematopoietic origin (Bradshaw J M. Cell Signal. 2010,22:1175-84). The Tec family includes TEC, Bruton's tyrosine kinase (BTK), inducible T-cell kinase (ITK), resting lymphocyte kinase (RLK/TXK), and bone marrow-expressed kinase (BMX/ETK).

BTK is important in B-cell receptor signaling and regulation of B-cell development and activation (Khan, W. N. et al. Immunity, 1995,3:283-299 and Satterthwaite A B et al. Immunol. Rev. 2000, 175: 120-127). Additionally, BTK is important in Fc receptor signaling due to immune complex deposition (Ellmeier W. et al. FEBS J. 2011, 278:1990-2000). Mutation of the gene encoding BTK in humans leads to X-linked agammaglobulinemia which is characterized by reduced immune function, including impaired maturation of B cells, decreased levels of immunoglobulin and peripheral B cells, diminished T-cell independent immune response (Rosen F S et al., N Engl. J. Med., 1995, 333:431-440; and Lindvall J M et al. Immunol. Rev. 2005, 203:200-215). BTK is activated by Src-family kinases and phosphorylates PLC gamma leading to effects on B-cell function and survival. Additionally, BTK is important for cellular function of mast cells, macrophage and neutrophils suggesting that BTK inhibition would be effective in treatment of diseases mediated by these and related cells including inflammation, bone disorders, and allergic disease (Kawakami Y. et al., J Leukoc Biol. 1999; 65(3):286-90).

BTK inhibition is also important in survival of lymphoma cells (Herman S E M. Blood, 2011, 117:6287-6289) suggesting that inhibition of BTK may be useful in the treatment of lymphomas and other cancers (Uckun F M, Int Rev Immunol. 2008; 27(1-2):43-69). Stromal cells within solid tumors include tumor associated lymphocytes and myeloid cells express TEC family kinases, particularly BTK (Stiff A. et al. Cancer Res. 76; 2125-2136). Regulatory B and T lymphocytes, myeloid derived suppressor cells (and tissue resident macrophages, dendritic cells and mast cells may provide stromal support and reduce innate and adaptive immune surveillance against transformed cells. Thus, inhibition of TEC family kinases, particularly BTK, is expected be beneficial in the treatment of solid tumors. As such, inhibitors of BTK and related kinases are of great interest as anti-inflammatory as well as anti-cancer agents. BTK is also important for platelet function and thrombus formation suggesting that BTK-selective inhibitors may prove to be useful antithrombotic agents (Liu J. Blood, 2006, 108:2596-603).

In addition BTK is expressed in HIV infected T-cells and treatment with BTK inhibitors sensitizes infected cells to apoptotic death and results in decreased virus production (Guendel I et al. J Neurovirol. 2015; 21:257-75). Accordingly, BTK inhibitors may be useful in the treatment of HIV-AIDS and other viral infections.

BMX, another Tec family member which has roles in inflammation, cardiovascular disease, and cancer (Cenni B. et al. Int Rev Immunol. 2012, 31: 166-173) is also important for self-renewal and tumerogenic potential of glioblastoma stem cells (Guryanova O A et al. Cancer Cell Cancer Cell 2011, 19:498-511). As such, BMX inhibitors may be useful in the treatment of various diseases including cancer, cardiovascular disease and inflammation.

### SUMMARY OF THE INVENTION

The present invention relates to a novel family of kinases inhibitors. Compounds of this class have been found to have inhibitory activity against members of the TEC kinase family, particularly BTK.

One aspect of the present invention is directed to a compound of Formula I:

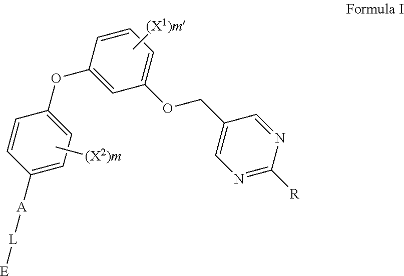

or a pharmaceutically acceptable salt, solvate, solvate of salt, stereoisomer, tautomer, isotope, prodrug, complex or biologically active metabolite thereof,

wherein

X¹ and X² are independently selected from hydrogen and halogen;

m is an integer from 0 to 4;

m′ is an integer from 0 to 4;

R is selected from hydrogen and methyl;

A is selected from:
1)

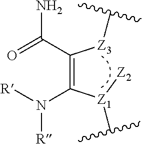

the dashed line is independently an optional bond;
R′ and R″ are independently selected from hydrogen, substituted or unsubstituted alkyl, substituted or unsubstituted heteroalkyl, substituted or unsubstituted cycloalkyl, substituted or unsubstituted heterocyclyl, substituted or unsubstituted aryl, substituted or unsubstituted heteroaryl, substituted or unsubstituted aralkyl or substituted or unsubstituted heteroaralkyl;
Z₁ and Z₃ are independently selected from C or N;
Z₂ is selected from N or CR¹; wherein R¹ is selected from hydrogen, halogen, substituted or unsubstituted alkyl, substituted or unsubstituted heteroalkyl, substituted or unsubstituted cycloalkyl, substituted or unsubstituted heterocyclyl, substituted or unsubstituted aryl, substituted or unsubstituted heteroaryl, substituted or unsubstituted aralkyl, or substituted or unsubstituted heteroaralkyl;
and
provided that at least one and no more than two of Z₁, Z₂ and Z₃ are simultaneously N; or
2)

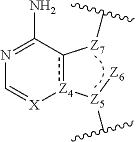

wherein the dashed lines are independently an optional bond;
Z₄, Z₅, and Z₇ are independently selected from C or N;
Z₆ is selected from N, C(O) or CR¹;
X is selected from N or CH;
provided that at least one and no more than two of Z₄, Z₅, Z₆ and Z₇ are simultaneously N; and
R¹ is selected from the group consisting of hydrogen, halogen, substituted or unsubstituted alkyl, substituted or unsubstituted heteroalkyl, substituted or unsubstituted cycloalkyl, substituted or unsubstituted heterocyclyl, substituted or unsubstituted aryl, substituted or unsubstituted heteroaryl, substituted or unsubstituted aralkyl, and substituted or unsubstituted heteroaralkyl;

L is independently selected from substituted or unsubstituted alkyl, substituted or unsubstituted heteroalkyl, substituted or unsubstituted cycloalkyl, substituted or unsubstituted heterocyclyl, substituted or unsubstituted aryl, substituted or unsubstituted heteroaryl, substituted or unsubstituted aralkyl, substituted or unsubstituted heteroaralkyl,

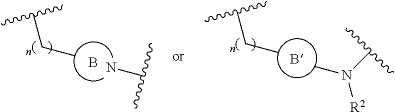

wherein
B is substituted or unsubstituted 3- to 8-membered nitrogen containing heterocyclic ring;
B′ is substituted or unsubstituted 3- to 8-membered cycloalkyl ring;
n is an integer from 0 to 1; and R² is selected from hydrogen and C₁₋₆ alkyl;

E is selected from the group consisting of:

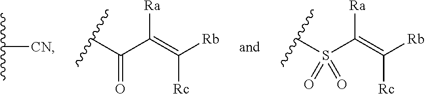

wherein
Ra, Rb and Rc are independently selected from hydrogen, halogen, —CN, substituted or unsubstituted alkyl, substituted or unsubstituted heteroalkyl, substituted or unsubstituted cycloalkyl, or substituted or unsubstituted heterocyclyl; or
Ra and Rb taken together with the carbon atoms to which they are attached form a 3- to 8-membered substituted or unsubstituted cycloalkyl ring, or form a 3-to 8-membered substituted or unsubstituted heterocyclic ring, and Rc is selected as above; or
Rb and Rc taken together with the carbon atom to which they are attached form a 3- to 8-membered substituted or unsubstituted cycloalkyl ring , or form a 3- to 8-membered heterocyclyc ring, and Ra is selected as above; or
Ra and Rb taken together with the carbon atoms to which they are attached form a triple bond and Rc is selected as above;

wherein A-L-E is

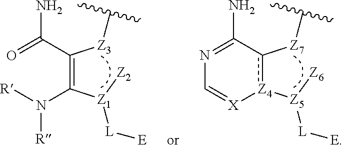

The compound of Formula I may just be the simple compound of Formula I in one embodiment.

The invention also encompasses compounds of Formula I which are solvate of salts, stereoisomers, tautomers, isotopically substituted variants, prodrugs, complexes or biologically active metabolites of the compounds represented by Formula I. Thus one aspect of the present invention is directed to a compound of Formula I:

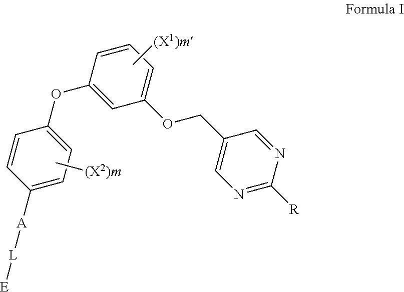

or a pharmaceutically acceptable salt, solvate, solvate of salt, stereoisomer, tautomer, isotope, prodrug, complex or biologically active metabolite thereof, wherein all of the substituents A, L, E, R, X¹, X² and m and m′ are as defined above.

An embodiment includes compounds of Formula I, wherein A is

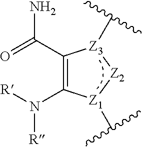

wherein the dashed line is independently an optional bond;

R′ and R″ are independently selected from hydrogen, substituted or unsubstituted alkyl, substituted or unsubstituted heteroalkyl, substituted or unsubstituted cycloalkyl, substituted or unsubstituted heterocyclyl, substituted or unsubstituted aryl, substituted or unsubstituted heteroaryl, substituted or unsubstituted aralkyl, or substituted or unsubstituted heteroaralkyl;

Z, and Z₃ are independently selected from C or N;

Z₂ is selected from N or CR¹; wherein R¹ is selected from hydrogen, halogen, substituted or unsubstituted alkyl, substituted or unsubstituted heteroalkyl, substituted or unsubstituted cycloalkyl, substituted or unsubstituted heterocyclyl, substituted or unsubstituted aryl, substituted or unsubstituted heteroaryl, substituted or unsubstituted aralkyl, or substituted or unsubstituted heteroaralkyl; and

provided that at least one and no more than two of Z₁, Z₂ and Z₃ are simultaneously N.

The present invention includes compounds of Formula I, wherein A is selected from the group consisting of:

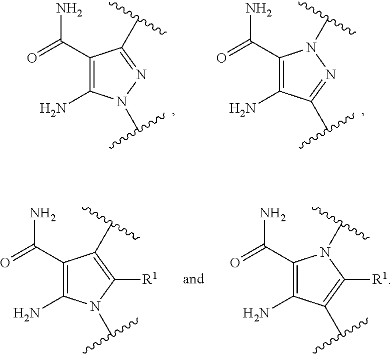

wherein R¹ is selected from hydrogen, halogen, substituted or unsubstituted alkyl, substituted or unsubstituted heteroalkyl, substituted or unsubstituted cycloalkyl, substituted or unsubstituted heterocyclyl, substituted or unsubstituted aryl, substituted or unsubstituted heteroaryl, substituted or unsubstituted aralkyl, or substituted or unsubstituted heteroaralkyl.

In an alternate embodiment the invention includes compounds of Formula I, wherein A is

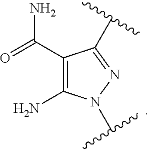 [SMILES: *c1nn(*)c(N)c1C(N)=O]

In an alternate embodiment the invention includes compounds of Formula I, wherein A is

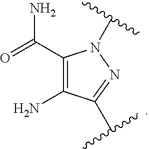 [SMILES: *c1nn(*)c(C(N)=O)c1N]

In an alternate embodiment the invention includes compounds of Formula I, wherein A is

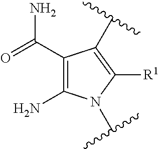

and R¹ is hydrogen.

In an alternate embodiment the invention includes compounds of Formula I, wherein A is

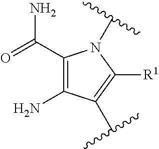

and R¹ is hydrogen.

An embodiment includes compounds of Formula I, wherein A is

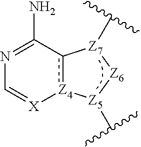

wherein the dashed lines are independently an optional bond;
Z₄, Z₅, and Z₇ are independently selected from C or N;
Z₆ is selected from N, C(O) or CR¹;
X is selected from N or CH; and
R¹ is selected from the group consisting of hydrogen, halogen, substituted or unsubstituted alkyl, substituted or unsubstituted heteroalkyl, substituted or unsubstituted cycloalkyl, substituted or unsubstituted heterocyclyl, substituted or unsubstituted aryl, substituted or unsubstituted heteroaryl, substituted or unsubstituted aralkyl, and substituted or unsubstituted heteroaralkyl;

provided that at least one and no more than two of Z₄, Z₅, Z₆ and Z₇ are simultaneously N.

The present invention includes compounds of Formula I, wherein A is selected from the group consisting of:

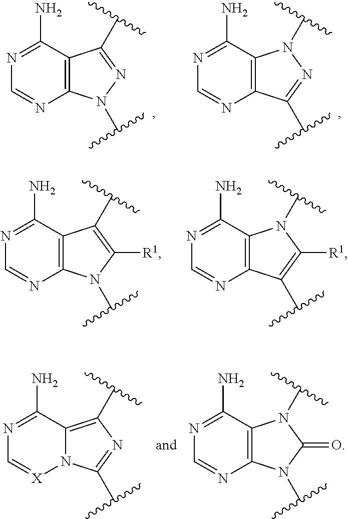

wherein
R¹ is selected from hydrogen, halogen, substituted or unsubstituted alkyl, substituted or unsubstituted heteroalkyl, substituted or unsubstituted cycloalkyl, substituted or unsubstituted heterocyclyl, substituted or unsubstituted aryl, substituted or unsubstituted heteroaryl, substituted or unsubstituted aralkyl, or substituted or unsubstituted heteroaralkyl;
X is selected from N or CH.

In an alternate embodiment the invention includes compounds of Formula I, wherein A is

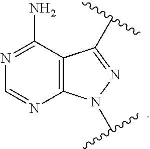 [SMILES: *c1nn(*)c2ncnc(N)c12]

In an alternate embodiment the invention includes compounds of Formula I, wherein A is

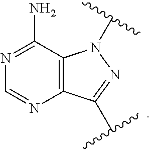 [SMILES: *c1nn(*)c2c(N)ncnc12]

In an alternate embodiment the invention includes compounds of Formula I, wherein A is

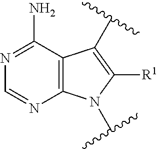

and R¹ is hydrogen.

In an alternate embodiment the invention includes compounds of Formula I, wherein A is

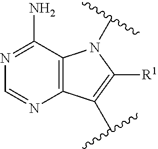

and R¹ is hydrogen.

In an alternate embodiment the invention includes compounds of Formula I, wherein A is

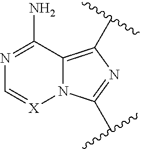

### and X is N.

In an alternate embodiment the invention includes compounds of Formula I, wherein A is

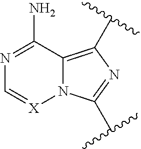

### and X is CH.

In an alternate embodiment the invention includes compounds of Formula I, wherein A is

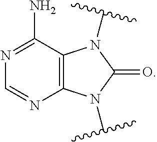 [SMILES: *n1c(=O)n(*)c2c(N)ncnc21]

In an alternate embodiment the invention includes compounds of Formula I, wherein R is methyl.

In an alternate embodiment the invention includes compounds of Formula I, wherein X¹ is F.

In an alternate embodiment the invention includes compounds of Formula I wherein X¹ is F and m′=1.

In an alternate embodiment the invention includes compounds of Formula I where X² is hydrogen.

In an embodiment the invention includes compounds of Formula I, wherein R is methyl, X¹ is F, m′=1, and X² is hydrogen.

The present invention includes compounds of Formula I, wherein L is selected from:

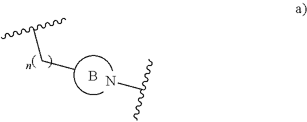

wherein B is substituted or unsubstituted 3- to 8-membered nitrogen containing heterocyclic ring;
n is an integer from 0 to 1; and

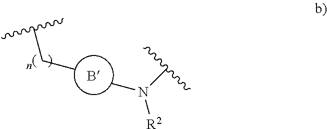

wherein B′ is substituted or unsubstituted 3- to 8-membered cycloalkyl ring;
n is an integer from 0 to 1; and
R² is selected from hydrogen and C₁₋₆ alkyl.

In an embodiment of the present invention includes compounds of Formula I, wherein L is independently selected from substituted or unsubstituted C₁₋₆ alkyl chain, substituted or unsubstituted heteroalkyl chain of 2 to 6 atoms, substituted or unsubstituted 3- to 8-membered cycloalkyl ring, substituted or unsubstituted 3- to 8-membered heterocyclyl ring, substituted or unsubstituted 5-, 6- and 7-membered aryl ring, substituted or unsubstituted 5-, 6- and 7-membered heteroaryl ring, substituted or unsubstituted aralkyl, substituted or unsubstituted heteroaralkyl.

The present invention includes compounds of Formula I, wherein L is selected from substituted or unsubstituted 3- to 8-membered cycloalkyl ring, substituted or unsubstituted 3- to 8-membered heterocyclyl ring, C₁₋₆ alkyl-(3- to 8-membered)heterocyclyl ring, C₁₋₆ alkyl-(3- to 8-membered) cycloalkyl ring —N(R²)—, or (3- to 8- membered)cycloalkyl ring-N(R²)—.

An embodiment of the present invention includes compounds of Formula I where L is:

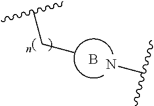

wherein B is substituted or unsubstituted 3- to 8-membered nitrogen containing heterocyclic ring, and n is an integer from 0 to 1.

Another embodiment of the present invention includes compounds of Formula I, wherein L is:

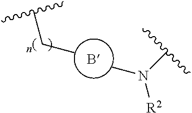

wherein B′ is substituted or unsubstituted 3- to 8-membered cycloalkyl ring; n is an integer from 0 to 1 and R² is selected from hydrogen and C₁₋₆ alkyl.

An embodiment of the present invention includes compounds of Formula I, wherein L-E is selected from the group consisting of:

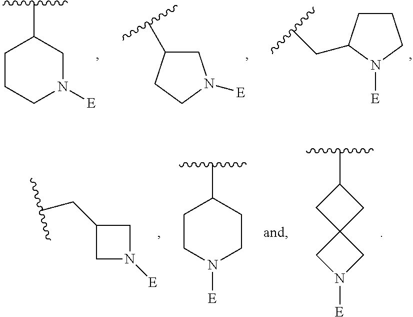

An embodiment of the present invention includes compounds of Formula I, wherein L-E is selected from the group consisting of:

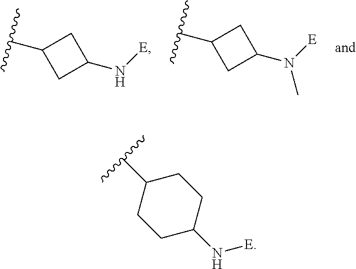

An embodiment of the present invention includes compounds of Formula I, wherein L-E is selected from the group consisting of:

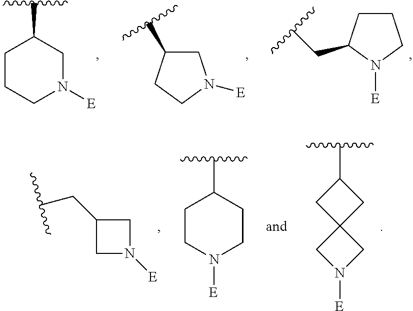

An embodiment of the present invention includes compounds of Formula I, wherein L-E is selected from the group consisting of:

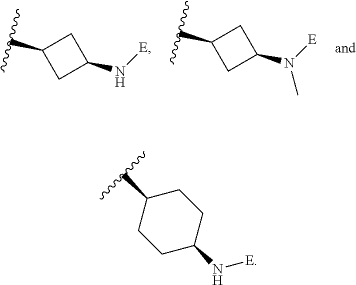

An embodiment includes compounds of Formula I, wherein E is —CN.

An embodiment includes compounds of Formula I, wherein E is selected from the group consisting of:

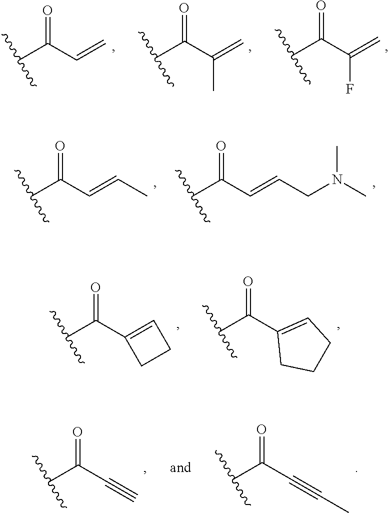

An embodiment includes compounds of Formula I, wherein E is

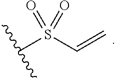 [SMILES: *S(=O)(=O)C=C]

In an embodiment of the present invention L-E is

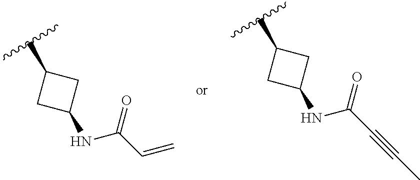

In an embodiment the invention includes compounds of Formula I, wherein Formula I is Formula I-1:

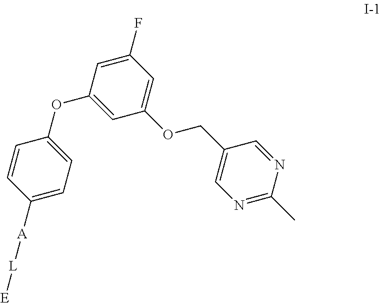

wherein A, L and E are defined above.

An additional embodiment of the present invention includes compounds of Formula I-1,

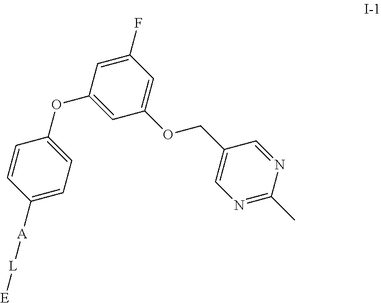

wherein A is selected from the group consisting of

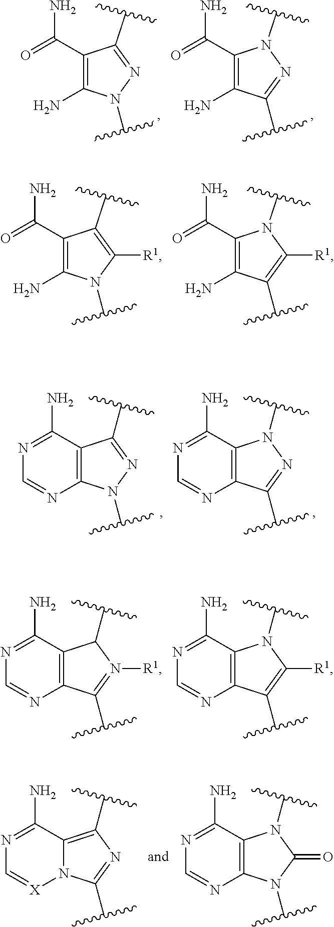

provided
R¹ is hydrogen and Xis N or CH; and

L-E is selected from the group consisting of

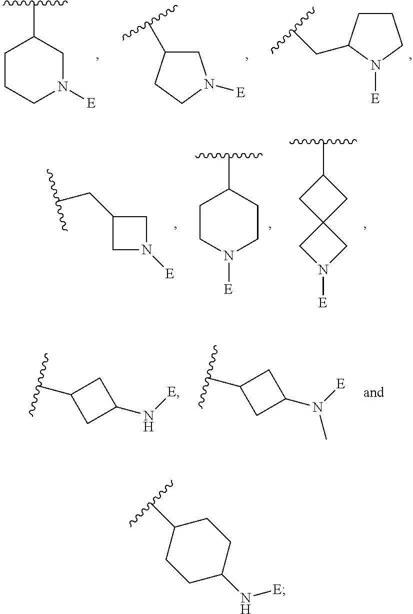

provided E is selected from the group consisting of:

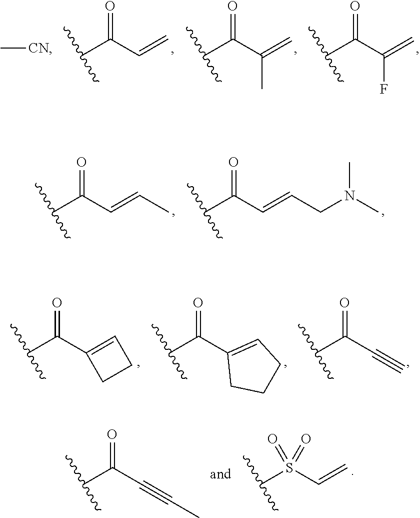

An embodiment of the present invention includes a compound having the chemical structure of Formula II, wherein Formula II is selected from the group comprising:

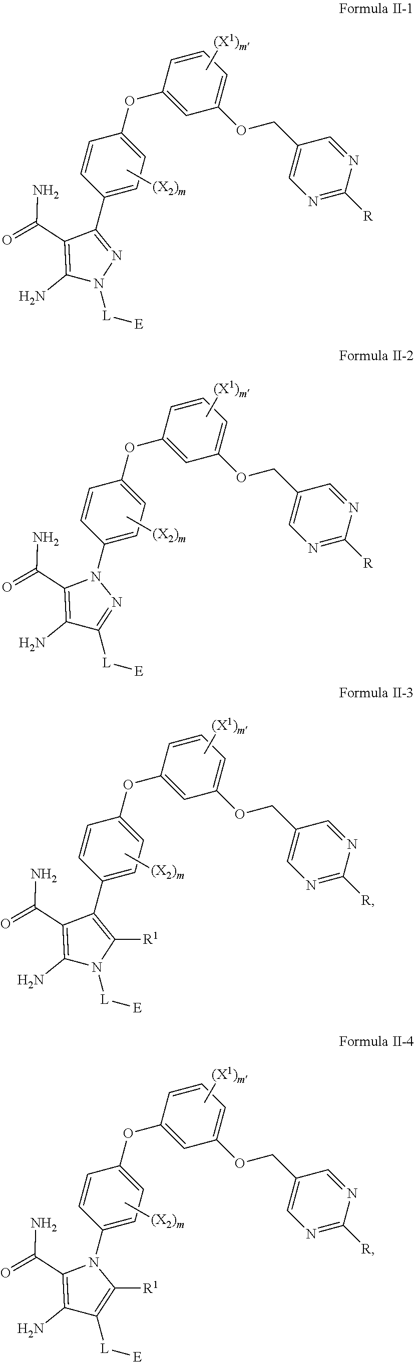

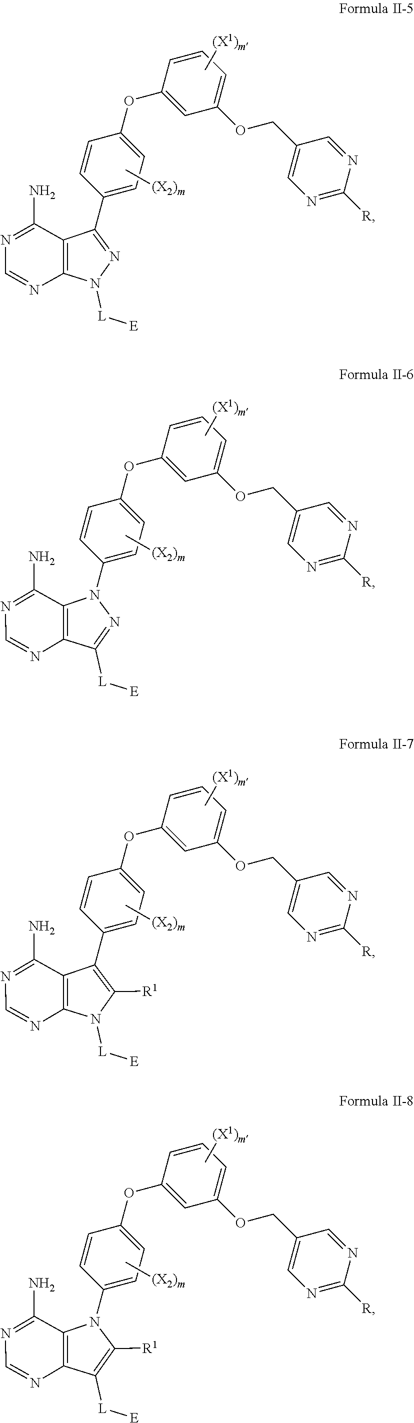

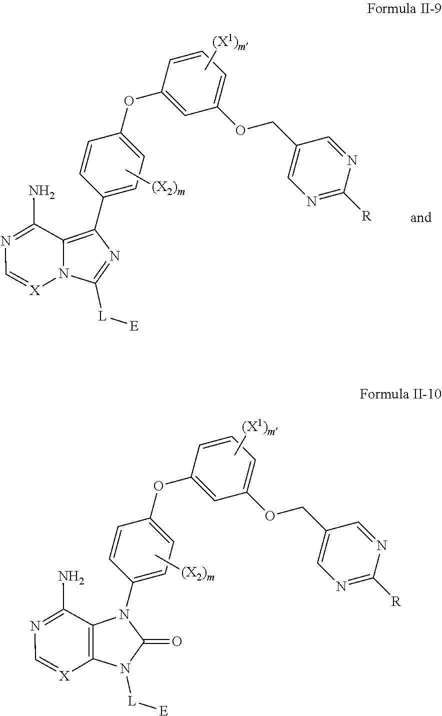

or a pharmaceutically acceptable salt, solvate, solvate of salt, stereoisomer, tautomer, isotope, prodrug, complex or biologically active metabolite thereof,

wherein:
X¹ and X² are independently selected from hydrogen and halogen;
m is an integer from 0 to 4;
m′ is an integer from 0 to 4;
R is selected from hydrogen and methyl;

L is selected from:

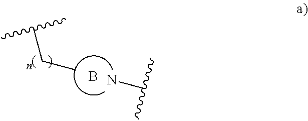

wherein B is substituted or unsubstituted 3- to 8-membered nitrogen containing heterocyclic ring;
n is an integer from 0 to 1; and

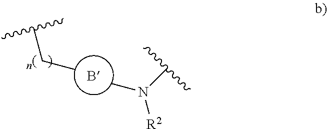

wherein B′ is substituted or unsubstituted 3- to 8-membered cycloalkyl ring;
n is an integer from 0 to 1; and R² is selected from hydrogen and methyl.

E is selected from the group consisting of:

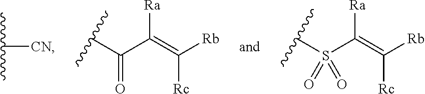

wherein
Ra, Rb and Rc are independently selected from hydrogen, halogen, —CN, substituted or unsubstituted alkyl, substituted or unsubstituted heteroalkyl, substituted or unsubstituted cycloalkyl, or substituted or unsubstituted heterocyclyl; or
Ra and Rb taken together with the carbon atoms to which they are attached form a 3- to 8-membered substituted or unsubstituted cycloalkyl ring, or form a 3-to 8-membered substituted or unsubstituted heterocyclic ring, and Rc is selected as above; or
Rb and Rc taken together with the carbon atom to which they are attached form a 3- to 8-membered substituted or unsubstituted cycloalkyl ring, or form a 3- to 8-membered heterocyclyc ring, and Ra is selected as above; or
Ra and Rb taken together with the carbon atoms to which they are attached form a triple bond, and Rc is selected as above.

The compound of Formula II is preferably the compound represented by Formula II or a pharmaceutically acceptable salt or solvate thereof. It may just be the simple compound of Formula II in one embodiment.

An embodiment of the present invention includes compounds of Formula II (preferably Formula II-5) or a pharmaceutically acceptable salt, solvate, solvate of salt, stereoisomer, tautomer, isotope, prodrug, complex or biologically active metabolite thereof, wherein L-E is selected from the group consisting of:

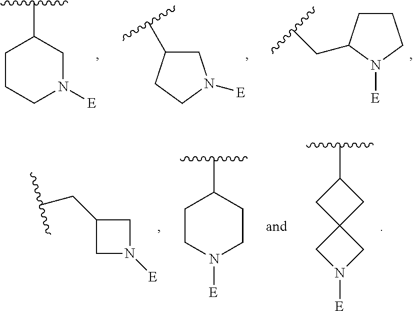

An embodiment of the present invention includes compounds of Formula II (preferably Formula II-5) or a pharmaceutically acceptable salt, solvate, solvate of salt, stereoisomer, tautomer, isotope, prodrug, complex or biologically active metabolite thereof, wherein L-E is selected from the group consisting of:

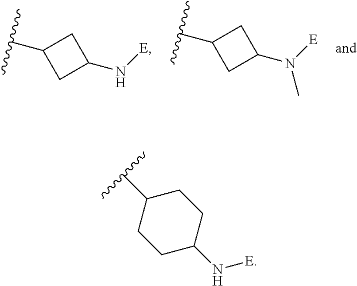

A preferred embodiment includes compounds of Formula II (preferably Formula II-5) or a pharmaceutically acceptable salt, solvate, solvate of salt, stereoisomer, tautomer, isotope, prodrug, complex or biologically active metabolite thereof, wherein L-E is selected from the group consisting of:

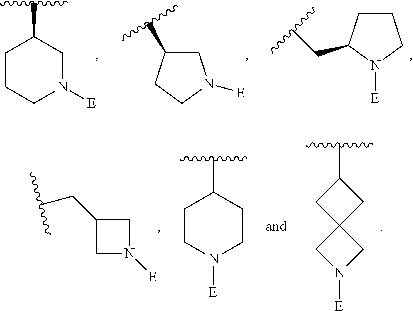

Another preferred embodiment includes compounds of Formula II (preferably Formula II-5) or a pharmaceutically acceptable salt, solvate, solvate of salt, stereoisomer, tautomer, isotope, prodrug, complex or biologically active metabolite thereof,

wherein L-E is selected from:

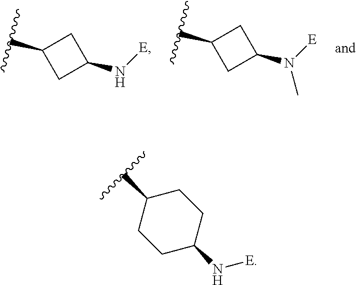

In an embodiment of the present invention L-E is

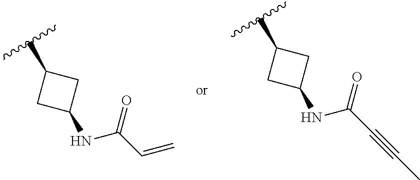

A preferred embodiment includes compounds of Formula II (preferably Formula II-5) or a pharmaceutically acceptable salt, solvate, solvate of salt, stereoisomer, tautomer, isotope, prodrug, complex or biologically active metabolite thereof, wherein E is —CN.

An embodiment includes compounds of Formula II (preferably Formula II-5) or a pharmaceutically acceptable salt, solvate, solvate of salt, stereoisomer, tautomer, isotope, prodrug, complex or biologically active metabolite thereof, wherein E is selected from the group consisting of:

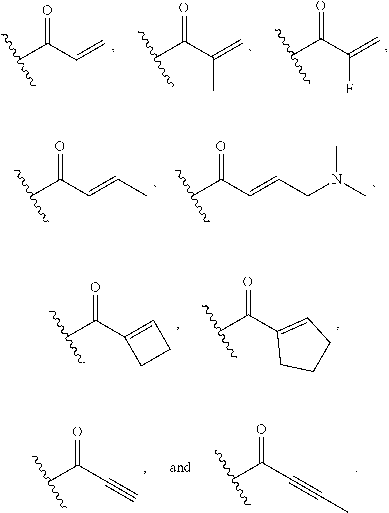

An embodiment of the present invention includes compounds of Formula II (preferably Formula II-5) or a pharmaceutically acceptable salt, solvate, solvate of salt, stereoisomer, tautomer, isotope, prodrug, complex or biologically active metabolite thereof, wherein E is

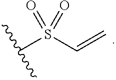 [SMILES: *S(=O)(=O)C=C]

A preferred embodiment of the present invention includes a compound having the chemical structure of Formula II-5

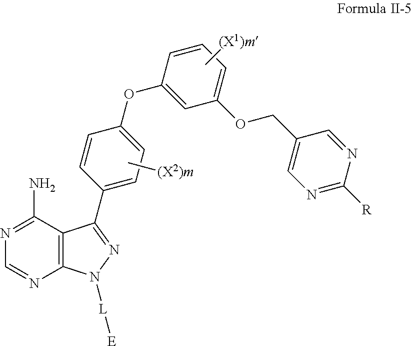

or a pharmaceutically acceptable salt, solvate, solvate of salt, stereoisomer, tautomer, isotope, prodrug, complex or biologically active metabolite thereof, wherein:
X¹ and X² are independently selected from hydrogen and halogen;
m is an integer from 0 to 4;
m′ is an integer from 0 to 4;
R is selected from hydrogen and methyl;

L is:

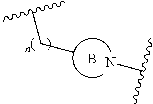

wherein B is substituted or unsubstituted 3- to 8-membered nitrogen containing heterocyclic ring; and n is an integer from 0 to 1; or

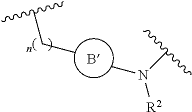

wherein B′ is substituted or unsubstituted 3- to 8-membered cycloalkyl ring; n is an integer from 0 to 1; and
R² is selected from hydrogen and methyl.

E is selected from the group selected from consisting of:

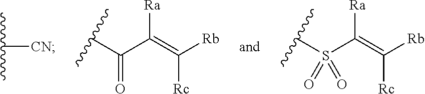

wherein
Ra, Rb and Rc are independently selected from hydrogen, halogen, —CN, substituted or unsubstituted alkyl, substituted or unsubstituted heteroalkyl, substituted or unsubstituted cycloalkyl, or substituted or unsubstituted heterocyclyl; or
Ra and Rb taken together with the carbon atoms to which they are attached form a 3- to 8-membered substituted or unsubstituted cycloalkyl ring, or form a 3-to 8-membered substituted or unsubstituted heterocyclic ring, and Rc is selected as above; or
Rb and Rc taken together with the carbon atom to which they are attached form a 3- to 8-membered substituted or unsubstituted cycloalkyl ring ,or form a 3-to 8-membered heterocyclic ring, and Ra is selected as above; or
Ra and Rb taken together with the carbon atoms to which they are attached form a triple bond, and Rc is selected as above.

An embodiment of the present invention includes compounds of Formula II-5 or a pharmaceutically acceptable salt, solvate, solvate of salt, stereoisomer, tautomer, isotope, prodrug, complex or biologically active metabolite thereof, wherein L-E is selected from the group consisting of:

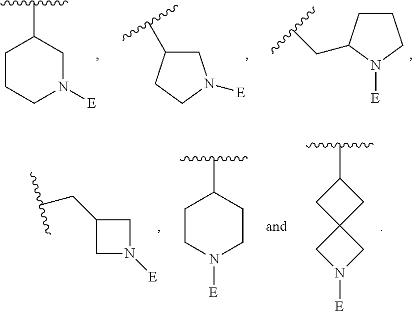

An embodiment of the present invention includes compounds of Formula II-5 or a pharmaceutically acceptable salt, solvate, solvate of salt, stereoisomer, tautomer, isotope, prodrug, complex or biologically active metabolite thereof, wherein L-E is selected from the group consisting of:

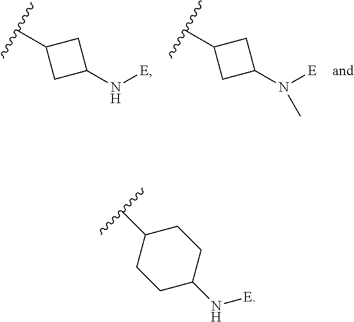

An embodiment of the present invention include compounds of Formula II-5 or a pharmaceutically acceptable salt, solvate, solvate of salt, stereoisomer, tautomer, isotope, prodrug, complex or biologically active metabolite thereof, wherein L-E is selected from the group consisting of:

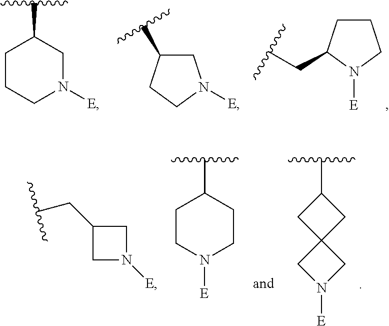

Another preferred embodiment includes compounds of Formula II-5 or a pharmaceutically acceptable salt, solvate, solvate of salt, stereoisomer, tautomer, isotope, prodrug, complex or biologically active metabolite thereof, wherein L-E is:

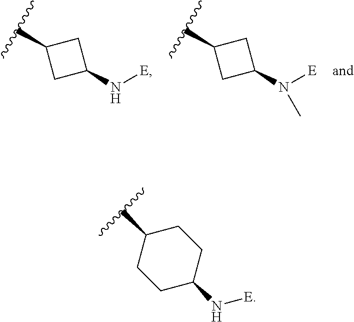

A preferred embodiment includes compounds of Formula II-5 or a pharmaceutically acceptable salt, solvate, solvate of salt, stereoisomer, tautomer, isotope, prodrug, complex or biologically active metabolite thereof, wherein E is —CN.

A preferred embodiment includes compounds of Formula II-5 or a pharmaceutically acceptable salt, solvate, solvate of salt, stereoisomer, tautomer, isotope, prodrug, complex or biologically active metabolite thereof, wherein E is selected from the group consisting of:

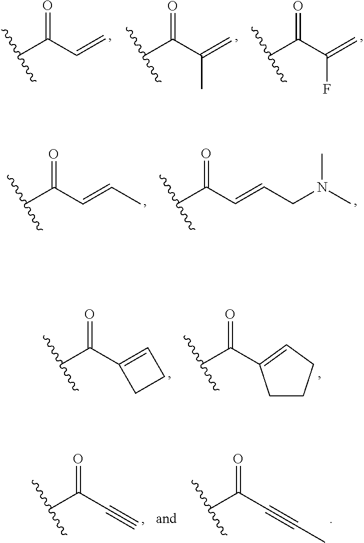

A preferred embodiment includes compounds of Formula II-5 or a pharmaceutically acceptable salt, solvate, solvate of salt, stereoisomer, tautomer, isotope, prodrug, complex or biologically active metabolite thereof, wherein E is

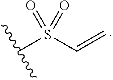 [SMILES: *S(=O)(=O)C=C]

A preferred embodiment includes compounds of Formula II-5 or a pharmaceutically acceptable salt, solvate, solvate of salt, stereoisomer, tautomer, isotope, prodrug, complex or biologically active metabolite thereof, wherein L-E is

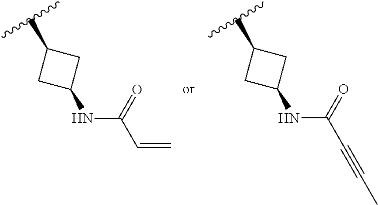

In an embodiment of the present invention a compound of Formula I (including Formula I-1) or Formula II (including compounds of Formula II-1 to II-10), or a pharmaceutically acceptable salt, solvate, solvate of salt, stereoisomer, tautomer, isotope, prodrug, complex or biologically active metabolite thereof, covalently binds to BTK, inhibiting the tyrosine kinase. In an alternate embodiment the compounds of the present invention form a covalent bond with BTK.

In further embodiment a compound of the present invention irreversibly inhibits the BTK to which it is covalently bound.

In an alternative embodiment, a compound of the present invention forms a covalent bond with a cysteine residue on BTK.

Another aspect of the present invention provides a pharmaceutical composition comprising a compound of Formula I (including Formula I-1) or Formula II (including compounds of Formula II-1 to II-10), or a pharmaceutically acceptable salt, solvate, solvate of salt, stereoisomer, tautomer, isotope, prodrug, complex or biologically active metabolite thereof, and at least one pharmaceutically acceptable carrier, diluent or excipient.

The pharmaceutical composition of the present invention is for use in prevention or treatment of cancer, autoimmune diseases, allergic diseases, inflammatory diseases, graft-versus-host disease, thromboembolic diseases, neurological disorders, viral infections, bone-related diseases or combinations thereof.

In an embodiment of the present invention a compound of Formula I (including Formula I-1) or Formula II (including Formula II-1 to II-10), or a pharmaceutically acceptable salt, solvate, solvate of salt, stereoisomer, tautomer, isotope, prodrug, complex or biologically active metabolite thereof suitable for use in therapy, wherein a subject is suffering of a disease, disorder or condition in which one or more TEC kinase family member, or BTK kinase activity is implicated.

In an embodiment of the present invention a compound of Formula I (including Formula I-1) or Formula II (including Formula II-1 to II-10), or a pharmaceutically acceptable salt, solvate, solvate of salt, stereoisomer, tautomer, isotope, prodrug, complex or biologically active metabolite thereof, is for use in the treatment or prevention of cancer, autoimmune diseases, allergic diseases, inflammatory diseases, neurological disorders, or viral infection in combination therapy.

In an embodiment of the present invention a compound of Formula I (including Formula I-1) or Formula II (including Formula II-1 to II-10), or a pharmaceutically acceptable salt, solvate, solvate of salt, stereoisomer, tautomer, isotope, prodrug, complex or biologically active metabolite thereof, is for use in therapy, further comprising at least one additional active pharmaceutical ingredient for the treatment or prevention of cancer, autoimmune diseases, allergic diseases, inflammatory diseases, neurological disorders or viral infection in combination therapy. The additional active pharmaceutical ingredient is selected from the group consisting of: steroids, leukotriene antagonists, anti-histamines, anti-cancer, anti-viral, anti-biotic agents, protein kinase inhibitors, immune modulators, checkpoint inhibitors and a combinations thereof, and wherein additional active pharmaceutical ingredient is administered together with the compounds of Formula I (including Formula I-1) or Formula II (including Formula II-1 to II-10) or a pharmaceutically acceptable salt, solvate, solvate of salt, stereoisomer, tautomer, isotope, prodrug, complex or biologically active metabolite thereof, as a single dosage form, or separately as part of a multiple dosage form.

In another aspect, the present invention relates to a compound of the invention as defined herein, or a pharmaceutically acceptable salt or solvate thereof, or a pharmaceutical composition as defined herein, for use in therapy or prevention of disease.

Compounds of the present invention, in any aspect or embodiment may be used in the treatment or prevention of cancer, autoimmune diseases selected from: rheumatoid arthritis, juvenile rheumatoid arthritis, osteoarthritis, ankylosing spondylitis, psoriatic arthritis, psoriasis vulgaris, pemphigus vulgaris, bullous pemphigoid, Sjogren's syndrome, systemic lupus erythromatosus, discoid SLE, lupus nephritis, antiphospholipidosis, whipple, dermatomyositis, polymyositis, autoimmune thrombocytopenia, idiopathic thrombocytopenia purpura, thrombotic thrombocytopenia purpura, autoimmune (cold) agglutinin disease, autoimmune hemolytic anemia, cryoglobulinemia, autoimmune vasculitis, ANCA-associated vasculitis, scleroderma, systemic sclerosis, multiple sclerosis, chronic focal encephalitis, Guillian-Barre syndrome, chronic fatigue syndrome, mononucleosis, neuromyelitis optica, autoimmune uveitis, Grave's disease, thyroid associated opthalmopathy, granulomatosis with microscopic polyangitis, Wegeners granulomatosis, idiopathic pulmonary fibrosis, sarcoidosis, idiopathic membranous nephropathy, IgA nephropathy, glomerulos clerosis , pancreatitis , type I diabetes or type II diabetes, allergic diseases, inflammatory diseases, neurological disorders or viral infection in combination therapy.

In another aspect, the present invention relates to a compound of the invention as defined herein, or a pharmaceutically acceptable salt or solvate thereof, or a pharmaceutical composition as defined herein, for use in the treatment of subjects suffering from a protein kinase mediated diseases or conditions.

Another aspect of the present invention provides a use of the compound of Formula I (including Formula I-1) or Formula II (including Formula II-1 to II-10) or a pharmaceutically acceptable salt or solvate thereof as an inhibitor of protein kinase, more particularly, as an inhibitor of BTK. In an embodiment the use is ex vivo, for example in vitro, such as an in vitro assay.

In another aspect, the present invention relates to the use of a compound of the invention as defined herein, or a pharmaceutically acceptable salt or solvate thereof, in the manufacture of a medicament for use in subjects for the treatment or prevention of protein kinase mediated diseases or conditions, for the treatment of cancer, autoimmune diseases, allergic diseases, inflammatory diseases, graft-versus-host disease, thromboembolic diseases, neurological disorders, viral infections, bone-related diseases or combinations thereof.

In another aspect, the present invention relates to a method of treating or prevention of a disease or condition associated with protein kinase activity, said method comprising administering to a subject a therapeutically effective amount of a compound of the invention as defined herein, or a pharmaceutically acceptable salt or solvate thereof, or a pharmaceutical composition as defined herein.

Another aspect of the present invention provides a compound, or a pharmaceutically acceptable salt or solvate thereof, or a pharmaceutical composition as defined herein, for use in the treatment of or prevention of diseases that involve BTK and\or other TEC kinases, i.e. diseases that involve B cells, T-cells and/or mast cells, for example, cancer, autoimmune diseases, allergic diseases, inflammatory diseases, graft-versus-host disease, thromboembolic diseases, bone-related diseases and the like.

A further aspect of the present invention provides the use of a compound, or a pharmaceutically acceptable salt or solvate thereof, in the manufacture of a medicament for use in the treatment or prevention of diseases that involve BTK and\or other TEC kinases, i.e. diseases that involve B cells, T-cells and mast cells, for example, cancer, autoimmune diseases, allergic diseases, inflammatory diseases, graft-versus-host disease, thromboembolic diseases, bone-related diseases and the like.

In another aspect, the present invention provides a method of treating or preventing a disease or condition, said method comprising administering to a subject a therapeutically effective amount of a compound of Formula I (including Formula I-1) or Formula II (including Formula II-1 to II-10), or a pharmaceutically acceptable salt or solvate thereof, or a pharmaceutical composition as defined herein. In a particular embodiment, the disease or conditions include allergic diseases, autoimmune diseases, inflammatory diseases, thromboembolic diseases, bone-related diseases, cancer, graft-versus-host disease, and the like.

Another aspect of the present invention provides a method of modulating kinase function, the method comprising contacting a cell with a compound of Formula I (including Formula I-1) or Formula II (including Formula II-1 to II-10) of the present invention in an amount sufficient to modulate the enzymatic activity of BTK, thereby modulating the kinase function. The method may be ex vivo, for example in vitro.

Another aspect of the present invention provides a method of inhibiting cell proliferation or survival in vitro or in vivo, said method comprising contacting a cell with an effective amount of a compound as defined herein, or a pharmaceutically acceptable salt or solvate thereof.

In one embodiment the present invention provides a method of producing a protein kinase inhibitory effect in a cell or tissue, said method comprising contacting the cell or tissue with an effective amount of a compound of Formula I (including Formula I-1) or Formula II (including Formula II-1 to II-10), or a pharmaceutically acceptable salt or solvate thereof.

In other embodiment, the present invention provides a method of producing a protein kinase inhibitory effect in vivo, said method comprising administering to a subject an effective amount of a compound, or a pharmaceutically acceptable salt or solvate thereof.

Another aspect of the present invention provides a method of modulating the target kinase function, comprising:
a) contacting a cell or a protein kinase with a compound of the present invention in an amount sufficient to modulate the target kinase function, thereby;
b) modulating the target kinase activity and signaling.

In yet another aspect, provided herein are methods of treating a disease treatable by inhibition of protein kinase in a patient which comprises administering to the patient a pharmaceutical composition comprising a compound disclosed herein and or a pharmaceutically acceptable salt thereof in a therapeutically effective amount and one or more pharmaceutically acceptable excipients. In one embodiment of this aspect the patient suffers from a disease or disorder that can be treated by kinase inhibition. The compound disclosed herein of Formula I (including Formula I-1) or Formula II (including Formula II-1 to II-10) and/or pharmaceutically acceptable salt thereof, can inhibit one or more kinases members of the TEC family of non-receptor protein kinases, including but not limited to ITK, BLK, BMX, BTK, JAK3, and/or TEC.

In another aspect the present invention provides a pharmaceutical combination comprising a compound of the present invention and at least one additional active pharmaceutical ingredient for the treatment or prevention of cancer, autoimmune diseases, allergic diseases, inflammatory diseases or viral infection in combination therapy.

In one embodiment the present invention provides a method of treatment wherein further comprising administering of a therapeutically effective amount of at least one additional active pharmaceutical ingredient for the treatment of cancer, autoimmune diseases, allergic diseases, inflammatory diseases, neurological disorders or viral infection in combination therapy. The additional active pharmaceutical ingredient is administered together with the compounds of Formula I or II as a single dosage form or separately as part of a multiple dosage form. The additional active pharmaceutical ingredient is selected from the group comprising: steroids, leukotriene antagonists, anti-histamines, anti-cancer, anti-viral, anti-biotic agents, protein kinase inhibitors or combinations thereof.

The administration of a compound of the present invention may be by any appropriate means known in the field, including systemic and localized administration. Prior to administration, the compounds may be formulated as compositions suitable for pharmaceutical or clinical use. Such compositions may comprise appropriate carriers or excipients, such as those for topical, inhalation, or systemic administration. The compound of the present invention may be administered alone or in combination with one or more pharmaceutically acceptable active for the treatment or prevention of a protein kinase mediated condition.

The present invention further provides a method of synthesizing a compound, or a pharmaceutically acceptable salt or solvate thereof, as defined herein.

Another aspect of the present invention provides a probe, the probe comprising a compound of Formula I labeled with a detectable label or an affinity tag. In other words, the probe comprises a residue of a compound of Formula I (including Formula I-1) or Formula II (including Formula II-1 to II-10) or a pharmaceutically acceptable salt or solvate thereof, covalently conjugated to a detectable label. Such detectable labels include, but are not limited to, a fluorescent moiety, a chemiluminescent moiety, a paramagnetic contrast agent, a metal chelate, a radioactive isotope-containing moiety and biotin.

All publications, patent applications, patents and other references mentioned herein are incorporated by references in their entirety.

Other features, objects, and advantages of the invention(s) disclosed herein will be apparent from the description and drawings, and from the claims.

### BRIEF DESCRIPTION OF THE DRAWINGS

FIG. 1. Is a structure of the reference compound for kinetic dilution and Arthus assays, it is disclosed in WO 2013/177668.

FIG. 2. Are example BTK inhibition curves obtained with compounds of the present invention.

FIG. 3. Are example dilution kinetic BTK results consistent with covalent inhibition of BTK by a compound of the present invention.

FIG. 4. Are example inhibition of IgM-mediated splenic cell proliferation results consistent with inhibition of BTK in immune cells.

FIG. 5. Are example inhibition of survival of TMD-8 lymphoma cells consistent with inhibition of BTK in lymphoma cells.

FIG. 6. Is a diagram demonstrating Arthus assay results. In vivo activity of the compounds of the present invention on the Fc receptor activation of immune complex acute vasculitis in the Arthus reverse passive anaphylaxis assay in rodents.

### DETAILED DESCRIPTION OF THE INVENTION

The present invention relates to novel kinase inhibitors. These compounds are found to have activity as inhibitors of protein kinases including members of the TEC kinase family including BTK, TEC, ITK/EMT/TSK, BMX and TXK/RLK. Most particularly, compounds of the present invention inhibit BTK enzyme and BTK-dependent cellular functions.

The term “compound” refers also to its pharmaceutically acceptable salt, solvate, solvate of salt, stereoisomer, tautomer, isotope, prodrug, complex or biologically active metabolite thereof.

In an embodiment of the present invention a compound of Formula I or Formula II, including compounds of Formula I-1 and Formula II-1 to II-10, or a pharmaceutically acceptable salt or solvate thereof, covalently binds to BTK, inhibiting the tyrosine kinase. In an alternate embodiment the compounds of the present invention form a covalent bond with the activated form of BTK. In further embodiment a compound of the present invention irreversibly inhibits the BTK to which it is covalently bound. In an alternative embodiment, a compound of the present invention forms a covalent bond with a cysteine residue on BTK.

Compounds of the present invention may be formulated into a pharmaceutical composition which comprises an effective amount of a compound of the instant invention with a pharmaceutically acceptable diluent or carrier.

The term “pharmaceutically effective amount” refers to any amount of the composition for use in humans or animals that is effective in treating or preventing a disease or condition associated with protein kinase activity.

Pharmaceutical Compositions

According to the present invention there is provided a pharmaceutical composition which comprises a compound of Formula I (including Formula I-1) or Formula II (including Formula II-1 to II-10) or a pharmaceutically acceptable salt or solvate thereof, in association with at least one pharmaceutically acceptable excipient, diluent or carrier.

The pharmaceutical compositions may be in a conventional pharmaceutical form suitable for oral administration (e.g., tablets, capsules, granules, powders and syrups), parenteral administration (e.g., injections (intravenous, intramuscular, or subcutaneous)), drop infusion preparations, inhalation, eye lotion, topical administration (e.g., ointment, cream), or suppositories. Regardless of the route of administration selected, the compounds may be formulated into pharmaceutically acceptable dosage forms by conventional methods known to those skilled in the art.

The phrase “pharmaceutically acceptable” is employed herein to refer to those ligands, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically acceptable carrier” as used herein means a pharmaceutically acceptable material, composition, or vehicle, such as a liquid or solid filler, diluent, excipient, solvent or encapsulating material. Each carrier must be acceptable in the sense of being compatible with the other ingredients of the formulation, including the active ingredient, and not injurious or harmful to the patient. Some examples of materials which can serve as pharmaceutically acceptable carriers include: (1) sugars, such as lactose, glucose, and sucrose; (2) starches, such as corn starch, potato starch, and substituted or unsubstituted β-cyclodextrin; (3) cellulose, and its derivatives, such as sodium carboxymethyl cellulose, ethyl cellulose, and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil, and soybean oil; (10) glycols, such as propylene glycol; (11) polyols, such as glycerin, sorbitol, mannitol, and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations. For oral formulations, “pharmaceutically acceptable carrier” such as cellulose, calcium silicate, corn starch, lactose, sucrose, dextrose, calcium phosphate, stearic acid, magnesium stearate, calcium stearate, gelatin, talc, surfactants, suspending agents, emulsifiers, diluents, and others may be used. For injectable formulations, “pharmaceutically acceptable carrier” such as water, saline, glucose solution, glucose solution analogs, alcohols, glycols, ethers (e.g., polyethylene glycol 400), oils, fatty acids, fatty acid esters, glycerides, surfactants, suspending agents, emulsifiers, and others may be used.

The term “pharmaceutically acceptable salt” refers to the relatively non-toxic, inorganic and organic acid addition salts of the compound(s). These salts can be prepared in situ during the final isolation and purification of the compound(s), or by separately reacting a purified compound(s) in its free base form with a suitable organic or inorganic acid, and isolating the salt thus formed. Representative salts include the hydrobromide, hydrochloride, sulfate, bisulfate, phosphate, nitrate, acetate, valerate, oleate, palmitate, stearate, laurate, benzoate, lactate, phosphate, tosylate, citrate, maleate, fumarate, succinate, tartrate, naphthylate, mesylate, glucoheptonate, lactobionate, laurylsulphonate salts, and amino acid salts, and the like (See, for example, Berge et al. (1977) “Pharmaceutical Salts”, J. Pharm. Sci. 66: 1-19).

The term “subject” or “patient” means a human or an animal subject for treatment.

The term “combination” within the meaning of this invention includes the simultaneous, sequential or separate use of the components or ingredients.

The pharmaceutical compositions of the present invention may be obtained by conventional procedures using conventional pharmaceutical excipients, well known in the art.

In other cases, the compounds of the present invention may contain one or more acidic functional groups and, thus, are capable of forming pharmaceutically acceptable salts with pharmaceutically acceptable bases. The term “pharmaceutically acceptable salts” in these instances refers to the relatively non-toxic inorganic and organic base addition salts of a compound(s). These salts can likewise be prepared in situ during the final isolation and purification of the compound(s), or by separately reacting the purified compound(s) in its free acid form with a suitable base, such as the hydroxide, carbonate, or bicarbonate of a pharmaceutically acceptable metal cation, with ammonia, or with a pharmaceutically acceptable organic primary, secondary, or tertiary amine. Representative alkali or alkaline earth salts include the lithium, sodium, potassium, calcium, magnesium, and aluminum salts, and the like. Representative organic amines useful for the formation of base addition salts include ethylamine, diethylamine, ethylenediamine, ethanolamine, diethanolamine, piperazine, and the like (see, for example, Berge et al., supra).

As used herein, the term “affinity tag” means a ligand or group, linked either to a compound of the present invention or to a protein kinase domain, that allows the conjugate to be extracted from a solution.

The term “spirocycle”, as used herein, refers to bicyclic rings system connected through just one atom. The rings can be different or identical. The connecting atom, also called spiroatom, is preferably a quaternary carbon. Spirocycle may be optionally substituted with one or more substituents as defined herein.

The term “alkyl”, as used herein, refers to a saturated hydrocarbon chain. Alkyl chains may be straight or branched. Alkyl chains may be optionally substituted with one or more substituents as defined herein. Representative alkyl groups include methyl, ethyl, propyl, (n-propyl and isopropyl) butyl (n-butyl, t-butyl and isobutyl), pentyl (n-pentyl and isopentyl), hexyl and the like. In certain preferred embodiments, alkyl substituents are lower alkyl groups, e.g., having from 1 to 6 carbon atoms.

The term “alkenyl”, as used herein, refers to an unsaturated hydrocarbon chain analogous in length and possible substitution to the “alkyl” described above, but that contain at least one double bond. Representative alkenyl groups include vinyl, propen-2-yl, crotyl, isopenten-2-yl, 1,3-butadien-2-yl, 2,4-pentadienyl, and 1,4-pentadien-3-yl. In certain preferred embodiments, alkenyl substituents are lower alkenyl groups, e.g., having from 2 to 6 carbon atoms.

The term “alkynyl”, as used herein, refers to an unsaturated hydrocarbon chain analogous in length and possible substitution to the “alkyl” described above, but that contain at least one triple bond. Representative alkynyl groups include ethynyl, 1- and 3-propynyl, and 3-butynyl. In certain preferred embodiments, alkynyl substituents are lower alkyl groups, e.g., having from 2 to 6 carbon atoms.

The term, “alkylene”, as used herein, refers to an alkyl group with two open valencies.

The term “heteroalkyl”, as used herein, refers to a saturated or partially saturated chain containing one to four heteroatoms selected from the group consisting of O, N and S, and wherein the nitrogen and sulfur atoms may optionally be oxidized and the nitrogen atom may optionally be quaternized. Heteroalkyl chains may be straight or branched. Heteroalkyl chains may be optionally substituted with one or more substituents as defined herein. The heteroatom(s) O, N and S may be placed at any interior position of the heteroalkyl group. Up to two heteroatoms may be consecutive.

The term “cycloalkyl”, as used herein, alternatively “carbocycle” and “carbocyclyl” refers to a saturated or partially saturated non-aromatic ring, more preferably 3- to 8-membered ring, in which each atom of the ring is carbon or; refers to a spirocycle where each ring is a saturated or partially saturated hydrocarbon ring and the Spiro atom is carbon. Cycloalkyl rings may be optionally substituted with one or more substituents as defined herein. The term “cycloalkyl”, “carbocycle” or “carbocyclyl” also include polycyclic ring systems having two or more cyclic rings in which two or more carbons are common to two adjoining rings wherein at least one of the rings is cycloalkyl, e.g., the other cyclic rings can be aryls, heteroaryls, and/or heterocyclyls. Representative cycloalkyl rings include cyclopropyl, cyclobutyl, cyclopentyl, cyclohexyl, 1-cyclohexenyl, 3-cyclohexen-1-yl, cycloheptyl, tetrahydronaphthyl, indanyl, adamantly and the like.

The term “heterocyclyl” alternatively “heterocyclic” and “heterocycloalkyl”, as used herein, refers to non-aromatic ring structures, more preferably 3- to 8-membered rings, whose ring structures include one to four heteroatoms or; refers to a spirocycle where the bicyclic rings system contains 1 to 4 heteroatoms. Heterocyclyl rings may be optionally substituted with one or more substituents as defined herein. The term “heterocyclyl” or “heterocyclic” also include polycyclic ring systems having two or more cyclic rings in which two or more carbons are common to two adjoining rings wherein at least one of the rings is heterocyclic, e.g., the other cyclic rings can be cycloalkyls, aryls and/or heteroaryls. Heterocyclyl groups include, for example, tetrahydrofuran, piperidine, piperazine, pyrrolidine, morpholine, lactones, lactams and the like.

The term “aryl”, as used herein, refers to 5-, 6-, and 7-membered aromatic rings in which each atom of the ring is carbon. Aryl rings may be optionally substituted with one or more substituents as defined herein. The term “aryl” also includes polycyclic ring systems having two or more cyclic rings in which two or more carbons are common to two adjoining rings wherein at least one of the rings is aryl, e.g., the other cyclic rings can be cycloalkyls, heteroaryls, and/or heterocyclyls. Aryl groups include, for example, benzene, naphthalene, phenanthrene, anthracene and the like.

The term “heteroaryl”, as used herein, refers to 5-, 6-, and 7-membered aromatic rings whose ring structures include one to four heteroatoms. Heteroaryl rings may be optionally substituted with one or more substituents as defined herein. The term “heteroaryl” also includes polycyclic ring systems having two or more cyclic rings in which two or more carbons are common to two adjoining rings wherein at least one of the rings is heteroaryl, e.g., the other cyclic rings can be cycloalkyls, aryls and/or heterocyclyls. Heteroaryl groups include, for example, pyrrole, furan, thiophene, imidazole, isoxazole, oxazole, thiazole, triazole, pyrazole, pyridine, pyrazine, pyridazine and pyrimidine, and the like.

The terms “polycyclyl” alternatively “polycyclic”, as used herein, refer to two or more rings (e.g., cycloalkyls, aryls, heteroaryls, and/or heterocyclyls) in which two or more carbons are common to two adjoining rings, e.g., the rings are “fused rings”. Polycyclyl rings may be optionally substituted with one or more substituents as defined herein.

The term “aralkyl”, as used herein, refers to an alkyl group substituted with an aryl group, for example —(CH₂)ₚ—Ar and p is an integer from 1 to 8 and Ar may be selected from any suitable aryl ring system, for example phenyl or napthyl. For example “aralkyl” may be benzyl.

The term “heteroaralkyl”, as used herein, refers to an alkyl group substituted with a heteroaryl group, for example —(CH₂)ₚ-Het wherein p is an integer from 1 to 8 and Het is any suitable heteroaryl ring system, such as those discussed in the above paragraphs.

The term “alkoxy”, as used herein, refers to an alkyl ether substituent, wherein the term alkyl is as defined above. Representative alkoxy groups include methoxy, ethoxy, propoxy, tert-butoxy and the like.

The term “ether”, as used herein, refers to an oxy group bridging two moieties linked at carbon atoms.

The term “alkoxyalkyl”, as used herein, refers to an alkyl group substituted with an alkoxy group, thereby forming ether.

The term “halo” or “halogen”, as used herein, refers to fluorine, chlorine, bromine and iodine.

The term “heteroatom”, as used herein, refers to an atom of any element other than carbon or hydrogen. Preferred heteroatoms are nitrogen, oxygen, and sulfur.

The term “hydrocarbon”, as used herein, refers to a group consisting entirely of carbon and hydrogen.

The term, “haloalkyl”, as used herein, refers to an alkyl substituent wherein one or more hydrogens are replaced by a halogen.

The term “carbonyl”, as used herein, when alone includes formyl —CH(O) and in combination is a —C(O) group.

The term “carboxyl”, alternatively “carboxy”, as used herein, refers to —C(O)OH or the corresponding “carboxylate” anion, such as in a carboxylic acid salt.

The term “acyl”, as used herein, refers to —C(O)R wherein R is alkyl, heteroalkyl, haloalkyl, cycloalkyl, heterocyclyl, aryl or heteroaryl as defined above. Representative acyl groups include acetyl, trifluoroacethyl, benzoyl, and the like.

The term “alkoxycarbonyl”, as used herein, refers to —C(O)OR wherein R is alkyl as defined above. Representative alkoxycarbonyl groups include methoxycarbonyl, ethoxycarbonyl, and the like.

The term “alkylthio”, as used herein, refers to a thioether —SR wherein R is alkyl as defined above. Representative alkylthio groups include methylthio, ethylthio and the like.

The term “sulfonate”, as used herein, refers to a salt or ester of a sulfonic acid —OSO₂R wherein R is alkyl, heteroalkyl, haloalkyl, cycloalkyl, heterocyclyl, aryl or heteroaryl as defined above. Representative sulfonate groups include mesylate, besylate, tosylate, and the like.

The term “sulfonyl”, as used herein, refers to —SO₂R wherein R is alkyl, heteroalkyl, haloalkyl, cycloalkyl, heterocyclyl, aryl or heteroaryl as defined above. Representative sulfonate groups include methylsufonyl, ethylsulfonyl, and the like.

The term “sulfamoyl”, as used herein, refers to —SO₂NH₂.

The term “sulfonamido”, as used herein, refers to —S(O)₂NRR′ wherein R and R′ are independently selected from alkyl, heteroalkyl, haloalkyl, cycloalkyl, heterocyclyl, aryl or heteroaryl as defined above. R and R′ may combine to form a heterocyclic ring.

The term “amino”, as used herein, refers to —NRR′ wherein R and R′ are independently selected from hydrogen, alkyl, heteroalkyl, haloalkyl, cycloalkyl, heterocyclyl, aryl or heteroaryl as defined above. R and R′ may combine to form a heterocyclic ring.

The term “amido” alternatively “amide”, as used herein, refers to —C(O)NRR′ wherein R and R′ are independently slected from hydrogen, alkyl, heteroalkyl, haloalkyl, cycloalkyl, heterocyclyl, aryl or heteroaryl as defined above. R and R′ may combine to form an heterocyclyl ring.

The term “substituted” refers to moieties having substituents replacing hydrogen on one or more atoms of the backbone. It will be understood that “substitution” or “substituted with” includes the implicit proviso that such substitution is in accordance with permitted valence of the substituted atom and the substituent, and that the substitution results in a stable compound, e.g., which does not spontaneously undergo transformation such as by rearrangement, cyclization, elimination, etc. As used herein, the term “substituted” is contemplated to include all permissible substituents of organic compounds. The permissible substituents can be one or more and the same or different for appropriate organic compounds. For purposes of this invention, the heteroatoms such as nitrogen may have hydrogen substituents and/or any permissible substituents of organic compounds described herein which satisfy the valences of the heteroatoms.

Substituents can include, for example, an alkyl, an alkenyl, an alkynyl, a haloalkyl, a heteroalkyl, a cycloalkyl, a heterocyclyl, an aryl, a heteroaryl, a halogen, a hydroxyl, a carbonyl , carboxyl, an alkoxycarbonyl, a formyl, or an acyl, a thiocarbonyl (such as a thioester, a thioacetate, or a thioformate), an alkoxy, a phosphoryl, a phosphate, a phosphonate, a phosphinate, an amino, an amido, an amidine, an imine, a cyano, a nitro, an azido, a sulfhydryl, an alkylthio, a sulfate, a sulfonate, a sulfamoyl, a sulfonamido, a sulfonyl. It will be understood by those skilled in the art that the substituents can themselves be substituted, if appropriate.

As used herein, the term “probe” means a compound of the invention which is labeled with either a detectable label or an affinity tag, and which is capable of binding, either covalently or non-covalently, to a protein kinase domain. When, for example, the probe is non-covalently bound, it may be displaced by a test compound. When, for example, the probe is bound covalently, it may be used to form cross-linked adducts, which may be quantified and inhibited by a test compound.

The term “prodrug” denotes a compound that is a drug precursor which, upon administration to a subject, is converted within the body into a compound of Formula I (including Formula I-1) or Formula II (including compounds of Formula II-1 to II-10) or a pharmaceutically acceptable salt or solvate thereof. Prodrugs of compounds of Formula I (including Formula I-1), Formula II, including compounds of Formula II-1 to II-10, or pharmaceutically acceptable salts or solvates thereof are within the scope of this disclosure.

Compounds of the present invention also include all isotopes of atoms present in the intermediates and/or final compounds. Isotopes include those atoms having the same atomic number but different mass numbers. For example, isotopes of hydrogen include deuterium and tritium.

Compounds of the present invention can be administered together with another drug as a concomitant medication to: (1) supplement and/or enhance the preventive and/or therapeutic effect of the either agent; (2) improve the kinetics/absorption, or reduce the dose of the either agent; and/or (3) mitigate side effects of either agent.

The concomitant medication that contains the other drug and the compound of the present invention can be administered as a formulation that combines both components therein or as a separate drug product. Administration thereof as a separate drug product includes both administration at the same time or administration at a different time.

Specific Abbreviations Used
AIDS Acquired Immune Deficiency Syndrome
ATP Adenosine Triphosphate
BLK B lymphocyte kinase
BMX Bone marrow-expressed kinase
BTK Bruton's Tyrosine Kinase
DMSO Dimethyl sulfoxide
EDTA Ethylenediaminetetraacetic acid
FCS Fetal Calf Serum
HIV Human Immunodeficiency Virus
HBV Hepatitis B virus
JAK3 Janus Kinase
ITK Interleukin-2 inducible T-cell kinase
NK/T-cell Natural killer T-cell
PBMC Peripheral blood mononuclear cells
PBS Phosphate buffered saline
RPMI Roswell Park Memorial Institute medium
RLK/TXK Resting Lymphocyte Kinase
TEC Tyrosine-protein Kinase
MS mass spectrometry
ml milliliter
μl microliter
mmol millimole
THF tetrahydrofuran
DMF dimethylformamide
DME Dimethoxyethane
NMP N-Methylpyrrolidone
MeOH methanol
EtOH ethanol
AcOH acetic acid
Na₂SO₃ Sodium sulfite
Cs₂CO₃ cesium carbonate
K₂CO₃ potassium carbonate
NH₄OH ammonium hydroxide
NaOH Sodium hydroxide
TEA triethylamine
DIPEA diisopropylethylamine
NaHCO₃ sodium bicarbonate
Boc₂O Di-tert-butyl dicarbonate
MgSO₄ magnesium sulfate
BrCN cyanogen bromide
TFA trifluoroacetic acid
HCl Hydrogen chloride
HATU (1-[Bis(dimethylamino)methylene]-1H-1,2,3-triazolo[4,5-b]pyridinium 3-oxid hexafluorophosphate)
EDC N-(3-Dimethylaminopropyl)-N′-ethylcarbodiimide
HOBt 1-Hydroxybenzotriazole
PPh₃ triphenyl phosphine
DIAD diisopropyl azodicarboxylate
CuI copper (I) iodide
NIS N-iodosuccinimide
NBS N-bromosuccinimide
PdCl₂(dppf) [1,1′-Bis(diphenylphosphino)ferrocene]dichloropalladium(II)
Pd(OAc₂) palladium (II) acetate
XPhos 2-Dicyclohexylphosphino-2′,4′,6′-triisopropylbiphenyl

General Synthetic Methods

In the description of the synthetic methods described below and in the referenced synthetic methods that are used to prepare the starting materials, it is to be understood that all proposed reaction conditions, including choice of solvent, reaction atmosphere, reaction temperature, duration of the experiment and workup procedures, can be selected by a person skilled in the art.

The following section describes general synthetic method(s) which may be useful in the preparation of compounds of the instant invention.

Compounds of Formula II-5 were prepared from commercially available starting materials as shown in Schemes A, B, C, D, E, F, G and H.

Intermediates A3 and B6 were prepared from commercially available starting material as shown in Schemes A and B.

Intermediates 1-c and 1-c′, 2-f, 3-b to 16-b were prepared as shown in Schemes 1 to 16 and Intermediates 26-c, 27-a, 28-b and 29-b were prepared as shown in Schemes 26 to 29.

Intermediate A3 is obtained in a 2 steps sequence starting from commercially available starting material A1. Halogenation of Al provides intermediate A2, amination of intermediate A2 provides intermediate A3. In an alternative method, amination of intermediate Al provide intermediate A2′, halogenation of intermediate A2′ provide intermediate A3.

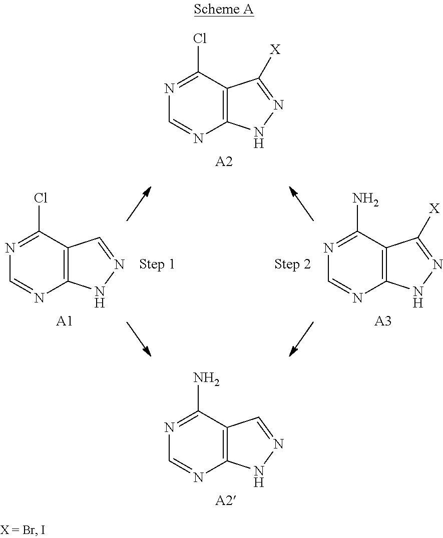

Metal-catalyzed nucleophilic aromatic substitution between commercially available starting materials aryl halide B1 and alcohol B2 provides ether intermediate B3. A second Ullmann reaction between intermediate B3 and commercially available starting material phenol derivative B4 provides intermediate B5. A metal-catalysed cross coupling reaction of halogen intermediate B5 with a tetraalkoxydiboron or dialkoxyhydroborane provides arylboronates intermediates of formula B6 (Ra′ and Rb′ are C₁-C₆ alkyl or Ra′ and Rb′ combine to form a cyclic boronic ester), the corresponding aryl boronic acids can be further obtained by hydrolysis (Ra′ and Rb′ are hydrogen).

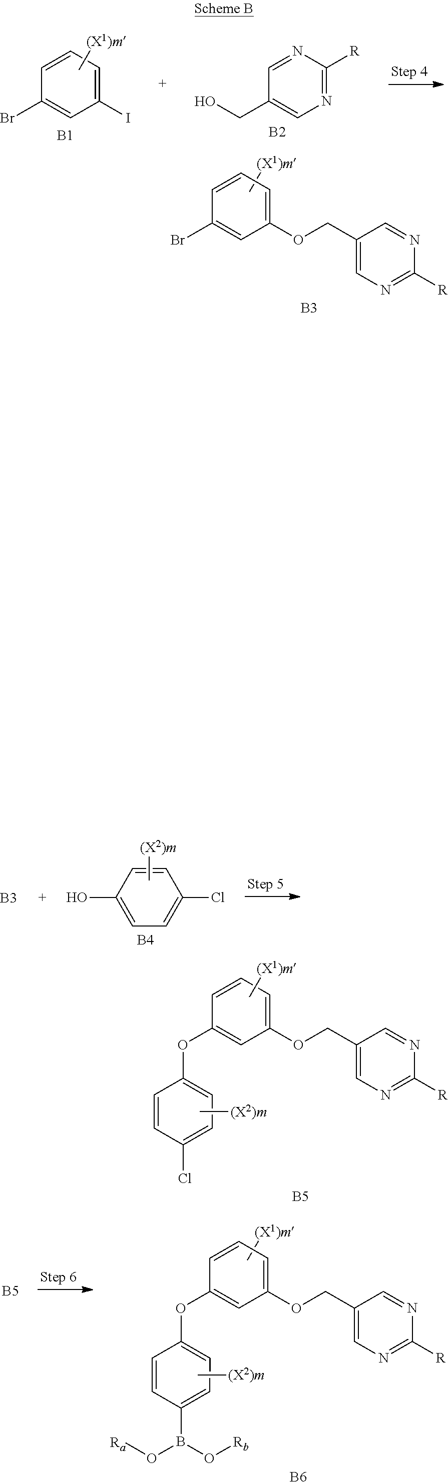

Compound of Formula II, where L is

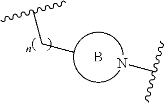

were prepared from commercially available starting materials as shown in Schemes C, D and E.

Intermediate A3 is coupled to intermediate C1 via Mitsunobu reaction to give intermediate C2. P is an appropriate amine protective group.

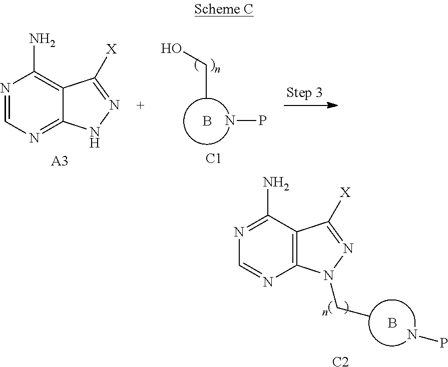

Metal catalyst cross coupling reaction of intermediate of formula C2 with a boronic acid or boronate ester of formula B6 under Suzuki coupling reaction conditions provide intermediate Dl. Deprotection of intermediate D1 provides intermediate D2.

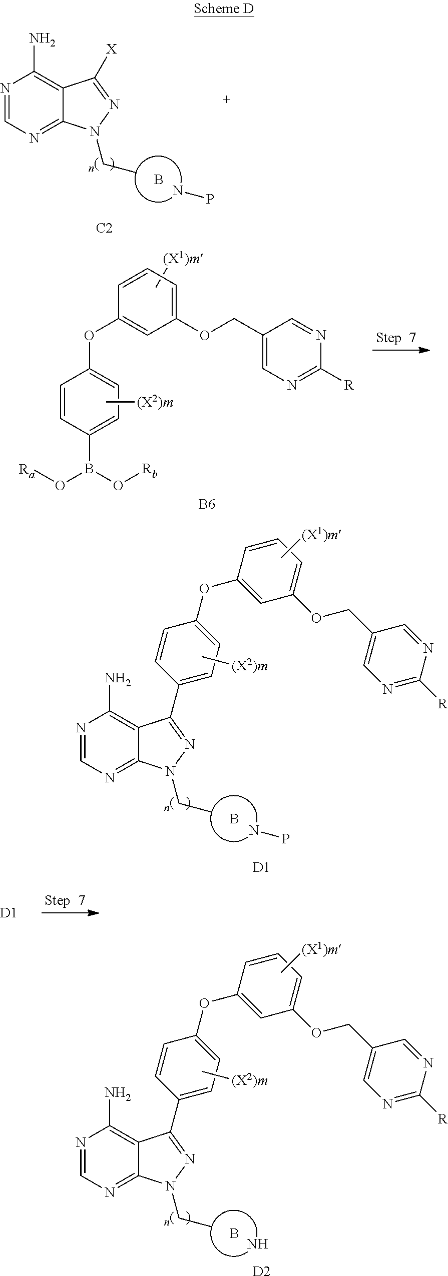

Compounds of Formula II are obtained from intermediate D2 by acylation, sulfonylation or by reacting intermediate D2 with cyanogen bromide.

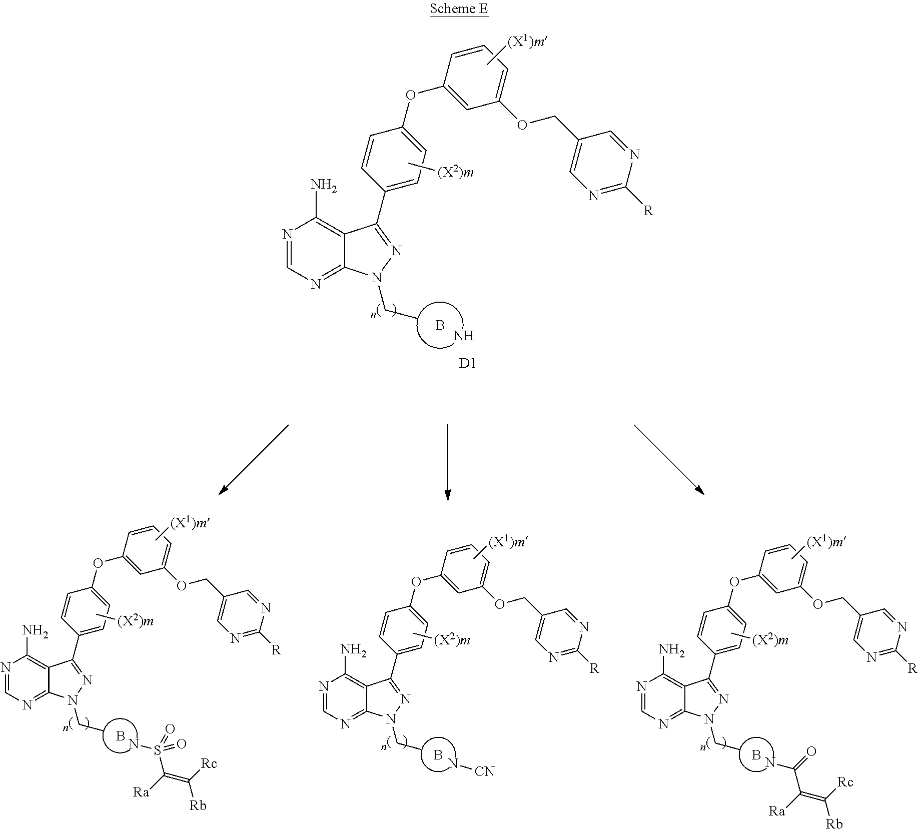

Compound of Formula II, where L is

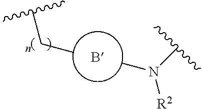

were prepared from commercially available starting materials as shown in Schemes F, G and H.

Intermediate F1 is coupled to intermediate A3 via Mitsunobu reaction to give intermediate F2. P is an appropriate amine protective group.

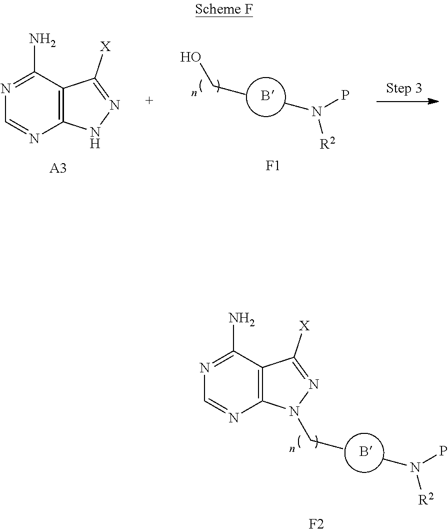

Metal catalyst cross coupling reaction of intermediate of formula F2 with a boronic acid or boronate ester of formula B6 under Suzuki coupling reaction conditions provide intermediate G1. Deprotection of intermediate G1 provides intermediate G2.

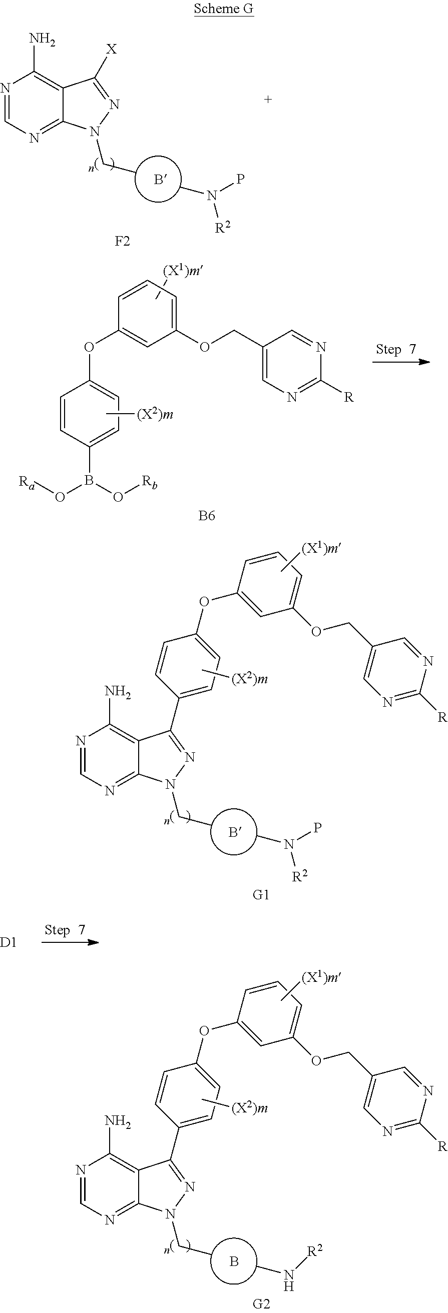

Compounds of Formula II are obtained from intermediate G2 by acylation, sulfonylation or by reacting intermediate D2 with cyanogen bromide.

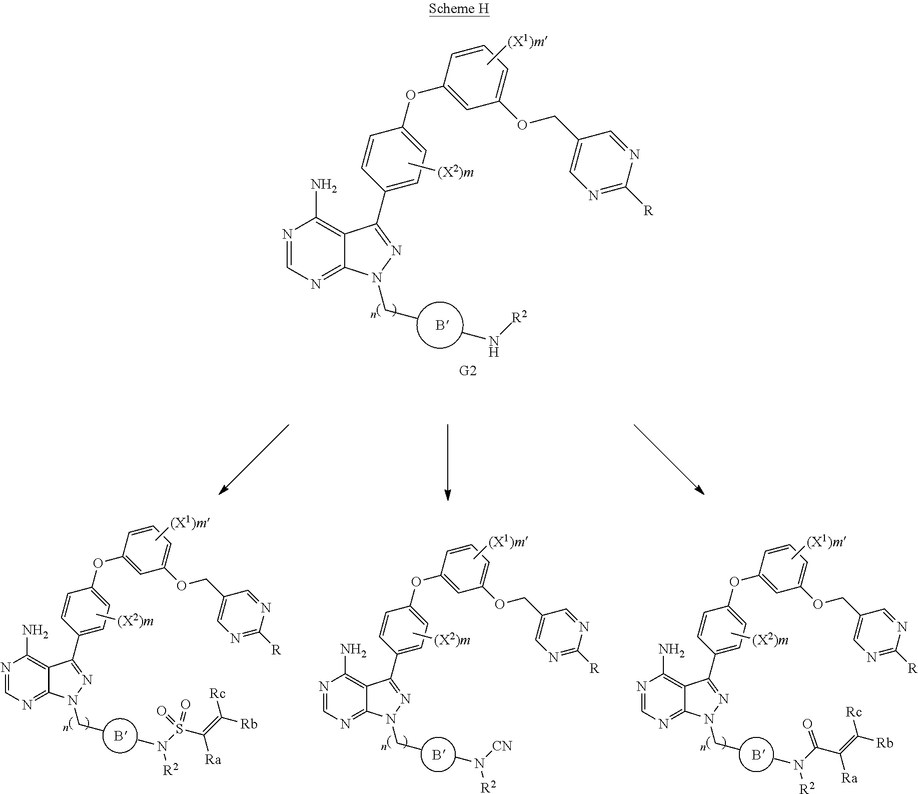

The following synthetic methods are intended to be representative of the chemistry used to prepare compound of Formula II of the present invention and are not intended to be limiting.

Intermediates 1-c and 1-c′, 2-f, 3-b to 16-b were prepared as shown in Schemes 1 to 16.

Synthesis of Intermediates 1-c and 1-c′

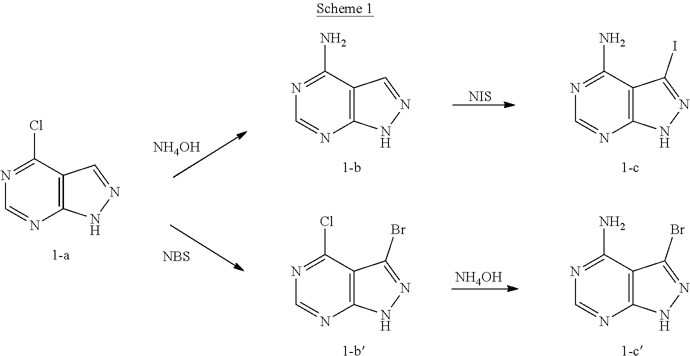

Step 1:

Intermediate 1-b

To a solution of intermediate 1-a (20.0 g, 129.0 mmol) in 2-propanol (90 ml) was added ammonium hydroxide (126 ml). The reaction was heated in a pressure vessel at 95° C. overnight then cooled to room temperature. Volatiles were removed under reduced pressure. The residue was triturated in water; a precipitate formed and was collected by filtration to provide intermediate 1-b as a white solid.

Intermediate 1-b′

To a solution of intermediate 1-a (10.0 g, 64.7 mmol) in DMF (162 ml) was added slowly added NBS (12.7 g, 71.2 mmol)). The reaction was stirred for 15 minutes at 0° C. and at room temperature overnight. Water was added; a precipitate formed and was collected by filtration then dried under vacuum to provide intermediate 1-b′ as a white solid.

Step 2:

Intermediate 1-c

To a solution of intermediate 1-b (14.2 g, 105.0 mmol) in DMF (120 ml) was added N-iodosuccinimide (35.5 g, 158.0 mmol) and the reaction was heated at 55° C. overnight and then cooled to room temperature. A saturated aqueous solution of Na₂SO₃ was added, a precipitate formed and was collected by filtration, washed with a saturated aqueous solution of Na₂SO₃ and then dried under vacuum to provide intermediate 1-c as a white solid.

Intermediate 1-c′

To a solution of intermediate 1-b′ (6.0 g, 25.7 mmol) in 2-propanol (36.0 ml) was added ammonium hydroxide (50.0 ml). The reaction was heated in a pressure vessel at 95° C. overnight and then cooled to room temperature. Volatiles were removed under reduced pressure. The residue was triturated in water; a precipitate formed and was collected by filtration, dried under vacuum to provide intermediate 1-c′ as a white solid.

Synthesis of intermediate 2-f:

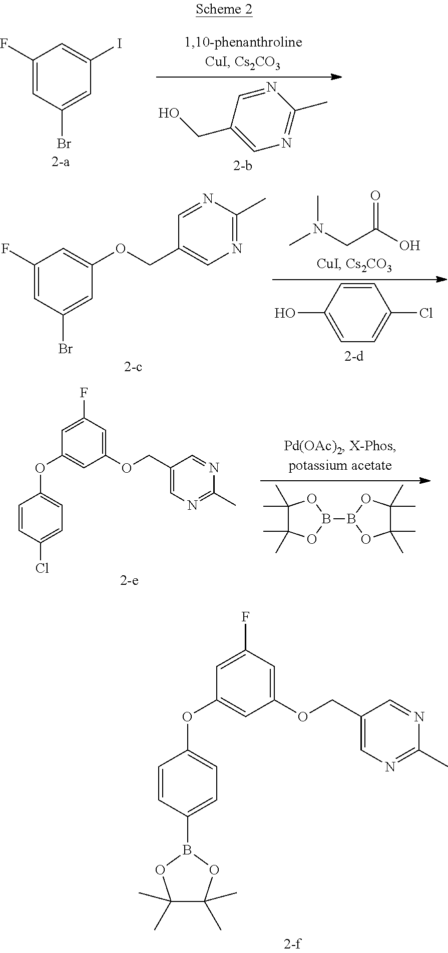

Step 1:

Intermediate 2-c

To a solution of 1-bromo-3-fluoro-5-iodobenzene 2-a (5.0 g, 16.62 mmol) in toluene (8.3 ml) was added (2-methylpyrimidin-5-yl)methanol 2-b (2.3 g, 18.3 mmol), 1,10-phenanthroline (599 mg, 3.3 mmol), copper (I) iodide (316 mg, 1.6 mmol) and cesium carbonate (7.6 g, 23.3 mmol). The reaction was stirred at 110° C. for 2 days and then cooled to room temperature, diluted with ethyl acetate and filtered over celite. A saturated aqueous solution of ammonium chloride was added to the filtrate, the organic layer was separated, and the aqueous phase was extracted twice with ethyl acetate. The combined organic extracts were washed with brine, dried over MgSO₄, filtered and concentrated under reduced pressure. Purification by silica gel chromatography provided intermediate 2-c as a beige solid.

Step 2:

Intermediate 2-e

A solution of intermediate 2-c (1.5 g, 5.0 mmol), 4-chlorophenol 2-d (681 mg, 5.3 mmol), N,N-dimethylglycine (1.5 g, 15.1 mmol), cesium carbonate (8.2 g, 25.2 mmol) and copper (I) iodide (961 mg, 5.0 mmol) in 1,4-dioxane (14.4 ml) was heated at 110° C. for 2 days and then cooled to room temperature. Ethyl acetate was added and the reaction was adsorbed on silica gel. Purification by silica gel chromatography provided intermediate 2-e as a colorless oil.

Step 3:

Intermediate 2-f

A degassed solution of intermediate 2-e (800 mg, 3.3 mmol), Palladium(II) acetate (37 mg, 0.17 mmol), potassium acetate (979 mg, 0.17 mmol) and X-Phos (158 mg, 0.33 mmol) was heated in a pressure vessel at 110° C. overnight and then cooled to room temperature. A saturated aqueous solution of ammonium chloride and ethyl acetate were added, the organic layer was separated, washed with brine, dried over MgSO₄, filtered and concentrated under reduced pressure. Purification by silica gel chromatography provided intermediate 2-f as a yellow solid.

Synthesis of Intermediate 3-b:

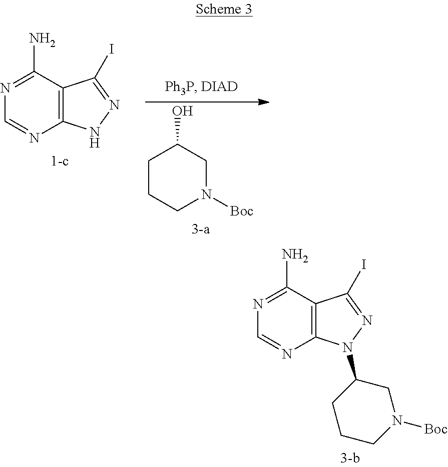 [SMILES: Nc1ncnc2[nH]nc(I)c12 ; CC(C)(C)OC(=O)N1CCC[C@H](O)C1 ; CC(C)(C)OC(=O)N1CCC[C@@H](n2nc(I)c3c(N)ncnc32)C1]

To a solution of intermediate 1-c (2.0 g, 7.7 mmol), intermediate 3-a (3.1 g, 15.3 mmol) and triphenylphosphine polymer-bound (7.7 g, ˜3 mmol/g triphenyl phosphine loading) in THF cooled to 0° C. was added DIAD (4.5 ml, 23.0 mmol) dropwise. After the addition was completed, the reaction was heated for 2 hours at 60° C. and then cooled to room temperature. The reaction was filtered and the filtrate was adsorbed on silica gel. Purification by silica gel chromatography provided intermediate 3-b as a white solid.

Synthesis of Intermediate 4-b:

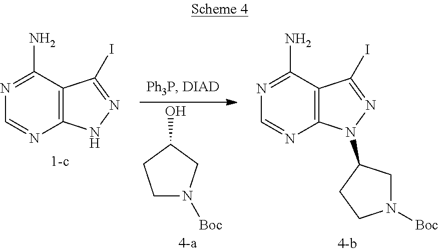 [SMILES: Nc1ncnc2[nH]nc(I)c12 ; CC(C)(C)OC(=O)N1CC[C@H](O)C1 ; CC(C)(C)OC(=O)N1CC[C@@H](n2nc(I)c3c(N)ncnc32)C1]

To a solution of intermediate 1-c (1.4 g, 5.3 mmol), intermediate 4-a (1.5 g, 8.0 mmol) and triphenylphosphine polymer-bound (3.6 g, ˜3 mmol/g triphenyl phosphine loading) in THF cooled to 0° C. was added DIAD (2.1 ml, 10.7 mmol) dropwise. After the addition was completed, the reaction was heated for 2 hours at 60° C. and then cooled to room temperature. The reaction was filtered and the filtrate was adsorbed on silica gel. Purification by silica gel chromatography provided intermediate 4-b as a white solid.

Synthesis of Intermediate 5-b:

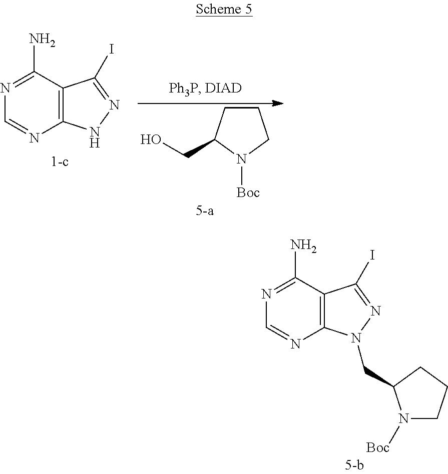 [SMILES: Nc1ncnc2[nH]nc(I)c12 ; CC(C)(C)OC(=O)N1CCC[C@@H]1CO ; CC(C)(C)OC(=O)N1CCC[C@@H]1Cn1nc(I)c2c(N)ncnc21]

To a solution of intermediate 1-c (1.0 g, 3.8 mmol), intermediate 5-a (850 mg, 4.2 mmol) and triphenylphosphine polymer-bound (1.7 g, ˜3 mmol/g triphenyl phosphine loading) in THF cooled to 0° C. was added DIAD (968 μl, 4.2 mmol) dropwise. After the addition was completed, the reaction was stirred at room temperature overnight. The reaction was filtered and the filtrate was adsorbed on silica gel. Purification by silica gel chromatography provided intermediate 5-b as a white solid.

Synthesis of Intermediate 6-b:

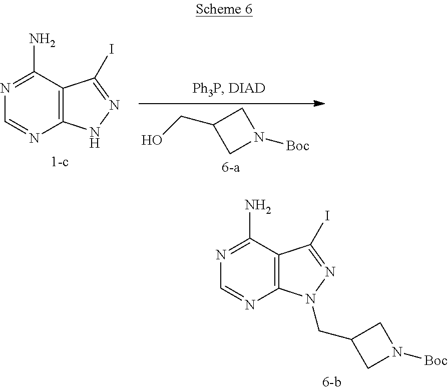 [SMILES: Nc1ncnc2[nH]nc(I)c12 ; CC(C)(C)OC(=O)N1CC(CO)C1 ; CC(C)(C)OC(=O)N1CC(Cn2nc(I)c3c(N)ncnc32)C1]

To a solution of intermediate 1-c (750 mg, 2.9 mmol), intermediate 6-a (538 mg, 2.9 mmol) and triphenylphosphine polymer-bound (2.8 g, ˜3 mmol/g triphenyl phosphine loading) in THF cooled to 0° C. was added DIAD (1.7 ml, 8.6 mmol) dropwise. After the addition was completed, the reaction was stirred at room temperature overnight. The reaction was filtered and the filtrate was adsorbed on silica gel. Purification by silica gel chromatography provided intermediate 6-b as a white solid.

Synthesis of Intermediate 7-b:

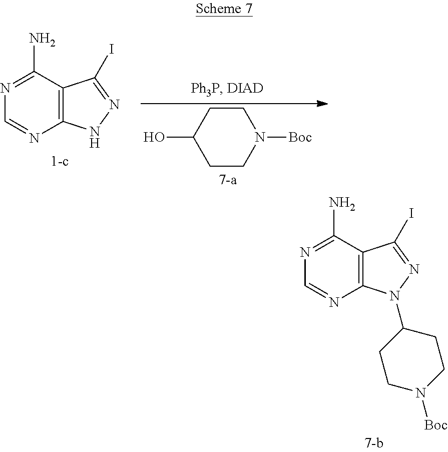 [SMILES: Nc1ncnc2[nH]nc(I)c12 ; CC(C)(C)OC(=O)N1CCC(O)CC1 ; CC(C)(C)OC(=O)N1CCC(n2nc(I)c3c(N)ncnc32)CC1]

To a solution of intermediate 1-c (750 mg, 2.9 mmol), intermediate 7-a (578 mg, 2.9 mmol) and triphenylphosphine polymer-bound (2.8 g, ˜3 mmol/g triphenyl phosphine loading) in THF cooled to 0° C. was added DIAD (1.7 ml, 8.6 mmol) dropwise. After the addition was completed, the reaction was stirred at room temperature overnight. The reaction was filtered and the filtrate was adsorbed on silica gel. Purification by silica gel chromatography provided intermediate 7-b as a white solid.

Synthesis of Intermediate 8-b:

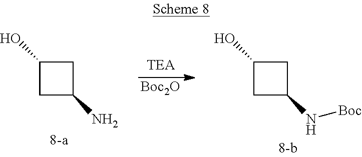 [SMILES: "Boc2O" ; N[C@H]1C[C@H](O)C1 ; CC(C)(C)OC(=O)N[C@H]1C[C@H](O)C1]

To a solution of in intermediate 26-a.HCl (10.0 g, 70.6 mmol) in ethanol (35 ml) were sequentially added TEA (35.0 ml) and Boc₂O (20.0 g, 31.3 mmol) and the reaction was stirred overnight at room temperature. Volatiles were removed under reduced pressure, ethyl acetate and water were added to the residue, the organic layer was separated, washed with a saturated aqueous solution of NaHCO₃ and brine, dried over MgSO₄, filtered and concentrated under reduced pressure to provide intermediate 8-b as a white solid.

Synthesis of Intermediate 9-b:

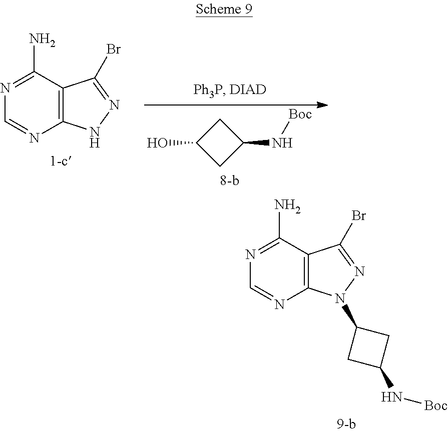 [SMILES: Nc1ncnc2[nH]nc(Br)c12 ; CC(C)(C)OC(=O)N[C@H]1C[C@H](O)C1 ; CC(C)(C)OC(=O)N[C@H]1C[C@@H](n2nc(Br)c3c(N)ncnc32)C1]

To a solution of intermediate 1-c′ (750 mg, 2.9 mmol), intermediate 8-b (1.75 g, 9.3 mmol) and triphenylphosphine polymer-bound (3.1 g, ˜3 mmol/g triphenyl phosphine loading) in THF cooled to 0° C. was added DIAD (1.8 ml, 9.3 mmol) dropwise. After the addition was completed, the reaction was stirred at room temperature overnight. The reaction was filtered and the filtrate was adsorbed on silica gel. Purification by silica gel chromatography provided intermediate 9-b as a white solid.

Synthesis of Intermediate 10-b:

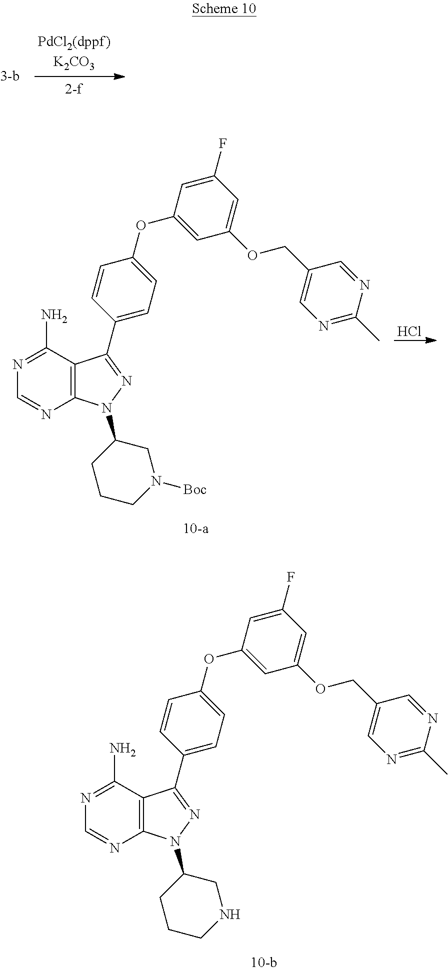 [SMILES: Cc1ncc(COc2cc(F)cc(Oc3ccc(-c4nn([C@@H]5CCCN(C(=O)OC(C)(C)C)C5)c5ncnc(N)c45)cc3)c2)cn1 ; Cl ; Cc1ncc(COc2cc(F)cc(Oc3ccc(-c4nn([C@@H]5CCCNC5)c5ncnc(N)c45)cc3)c2)cn1]

Step 1:Intermediate 10-a

To a degassed solution of intermediate 3-b (1.1 g, 2.8 mmol), intermediate 2-f (1.3 g, 2.9 mmol) and potassium carbonate (1.1 g, 8.3 mmol) in DME (14.8 ml) and water (3.7 ml) was added PdCl₂(dppf) (₂₀₃ mg, 0.3 mmol) and the reaction was heated in a pressure vessel at 105° C. for 2 hours and then cooled to room temperature. Ethyl acetate was added and the reaction was filtered over celite. A saturated aqueous solution of ammonium chloride was added to the filtrate, the organic layer was separated, washed with brine, dried over MgSO₄, filtered and concentrated under reduced pressure. Purification by silica gel chromatography provided intermediate 10-a as a white solid.

Step 2:Intermediate 10-b

To a solution of intermediate 10-a (1.5 g, 2.4 mmol) in 1,4-dioxane (10 ml) and methanol (1 ml) cooled to 0° C. was added a solution of 4N HCl in 1,4-dioxane (2.9 ml, 96 mmol). After the addition was completed the reaction was stirred for 1 hour at room temperature. Diethyl ether was added, a precipitate formed and was collected by filtration to provide intermediate 10-b.3HCl as a yellow solid.

Synthesis of Intermediate 11-b:

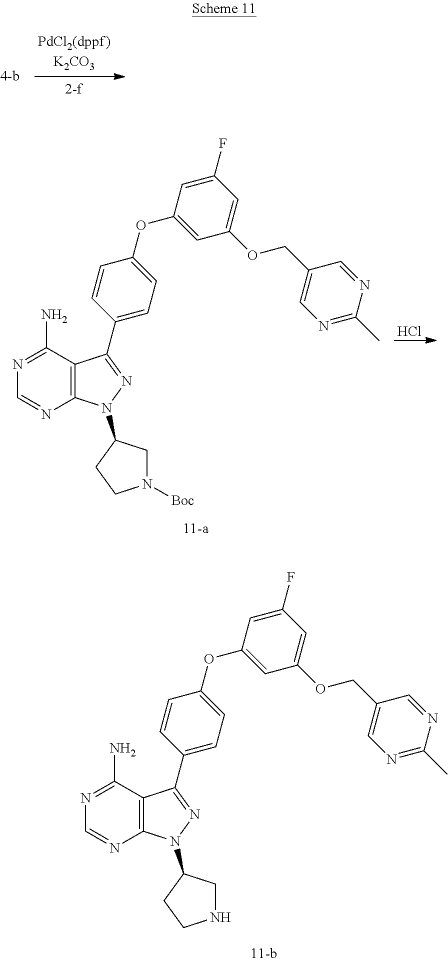 [SMILES: Cc1ncc(COc2cc(F)cc(Oc3ccc(-c4nn([C@@H]5CCN(C(=O)OC(C)(C)C)C5)c5ncnc(N)c45)cc3)c2)cn1 ; Cl ; Cc1ncc(COc2cc(F)cc(Oc3ccc(-c4nn([C@@H]5CCNC5)c5ncnc(N)c45)cc3)c2)cn1]

Step 1:Intermediate 11-a

To a degassed solution of intermediate 4-b (1.1 g, 2.5 mmol), intermediate 2-f (1.1 g, 2.6 mmol) and potassium carbonate (1.0 g, 7.5 mmol) in DME (13.4 ml) and water (3.3 ml) was added PdCl₂(dppf) (184 mg, 0.2 mmol) and the reaction was heated in a pressure vessel at 105° C. overnight and then cooled to room temperature. Ethyl acetate was added and the reaction was filtered over celite. A saturated aqueous solution of ammonium chloride was added to the filtrate, the organic layer was separated, washed with brine, dried over MgSO₄, filtered and concentrated under reduced pressure. Purification by silica gel chromatography provided intermediate 11-a as a white foam.

Step 2:Intermediate 11-b

To a solution of intermediate 11-a (1.5 g, 2.4 mmol) in 1,4-dioxane (10 ml) and methanol (1 ml) cooled to 0° C. was added a solution of 4N HCl in 1,4-dioxane (2.9 ml, 96 mmol). After the addition was completed the reaction was stirred for 1 hour at room temperature. Diethyl ether was added, a precipitate formed and was collected by filtration to provide intermediate 11-b.3HCl as a yellow solid. Intermediate 11-b.3HCl was suspended in dichloromethane and a saturated aqueous solution of NaHCO₃ was slowly added. After stirring for 15 minutes the organic layer was separated, washed with a saturated aqueous solution of NaHCO₃ and brine, dried over MgSO₄, filtered and concentrated under reduced pressure to provide intermediate 11-b as a yellow solid.

Synthesis of Intermediate 12-b:

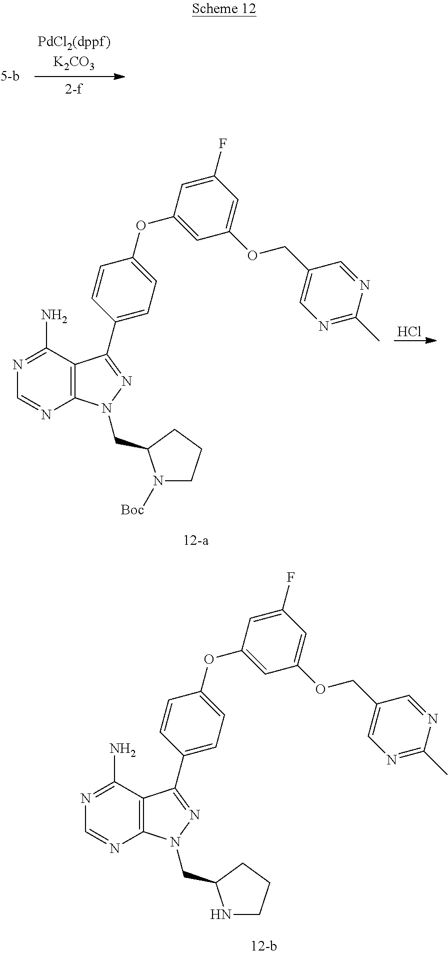 [SMILES: Cc1ncc(COc2cc(F)cc(Oc3ccc(-c4nn(C[C@H]5CCCN5C(=O)OC(C)(C)C)c5ncnc(N)c45)cc3)c2)cn1 ; Cl ; Cc1ncc(COc2cc(F)cc(Oc3ccc(-c4nn(C[C@H]5CCCN5)c5ncnc(N)c45)cc3)c2)cn1]

Step 1:

Intermediate 12-a

To a degassed solution of intermediate 5-b (1.7 g, 3.8 mmol), intermediate 2-f (1.7 g, 4.0 mmol) and potassium carbonate (1.6 g, 11.5 mmol) in DME (20.4 ml) and water (5.1 ml) was added PdCl₂(dppf) (₂₈₀ mg, 0.4 mmol) and the reaction was heated in a pressure vessel at 105° C. for 2 hours and then cooled to room temperature. Ethyl acetate was added and the reaction was filtered over celite. A saturated aqueous solution of ammonium chloride was added to the filtrate, the organic layer was separated, washed with brine, dried over MgSO₄, filtered and concentrated under reduced pressure. Purification by silica gel chromatography provided intermediate 12-a as a white foam.

Step 2:

Intermediate 12-b

To a solution of intermediate 12-a (2.0 g, 3.2 mmol) in 1,4-dioxane (10 ml) and methanol (1 ml) cooled to 0° C. was added a solution of 4N HCl in 1,4-dioxane (3.4 ml, 128 mmol). After the addition was completed the reaction was stirred for 1 hour at 0° C. Diethyl ether was added, a precipitate formed and was collected by filtration to provide intermediate 12-b.3HCl as a yellow solid.

Synthesis of Intermediate 13-b:

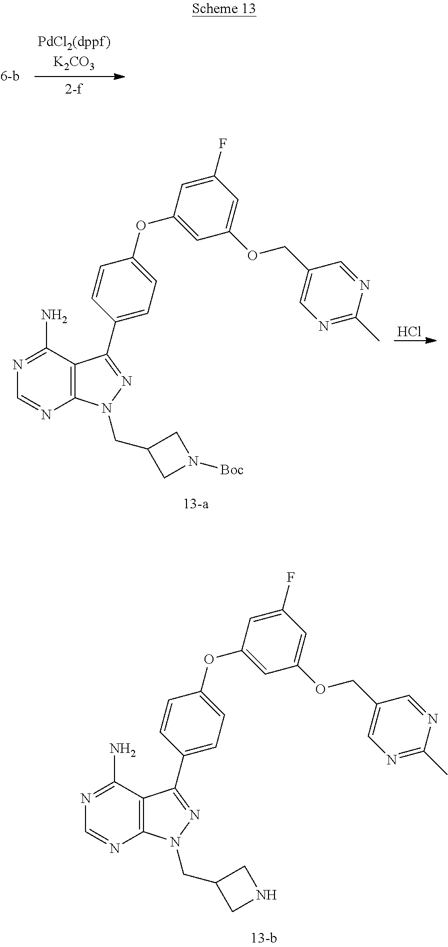 [SMILES: Cc1ncc(COc2cc(F)cc(Oc3ccc(-c4nn(CC5CN(C(=O)OC(C)(C)C)C5)c5ncnc(N)c45)cc3)c2)cn1 ; Cl ; Cc1ncc(COc2cc(F)cc(Oc3ccc(-c4nn(CC5CNC5)c5ncnc(N)c45)cc3)c2)cn1]

Step 1:

Intermediate 13-a

To a degassed solution of intermediate 6-b (360 mg, 0.8 mmol), intermediate 2-f (402 mg, 0.9 mmol) and potassium carbonate (347 mg, 2.5 mmol) in DME (4.5 ml) and water (1.1 ml) was added PdCl₂(dppf) (₆₁ mg, 0.08 mmol) and the reaction was heated in a pressure vessel at 105° C. overnight and then cooled to room temperature. Ethyl acetate was added and the reaction was filtered over celite. A saturated aqueous solution of ammonium chloride was added to the filtrate, the organic layer was separated, washed with brine, dried over MgSO₄, filtered and concentrated under reduced pressure. Purification by silica gel chromatography provided intermediate 13-a as a white foam.

Step 2:

Intermediate 13-b

To a solution of intermediate 13-a (513 mg, 0.8 mmol) in 1,4-dioxane (5 ml) and methanol (1 ml) cooled to 0° C. was added a solution of 4N HCl in 1,4-dioxane (4.2 ml, 16.7 mmol). After the addition was completed the reaction was stirred for 1 hour at 0° C. Volatiles were removed under reduced pressure. Diethyl ether was added, a precipitate formed and was collected by filtration to provide intermediate 13-b.3HCl as a white solid.

Synthesis of Intermediate 14-b:

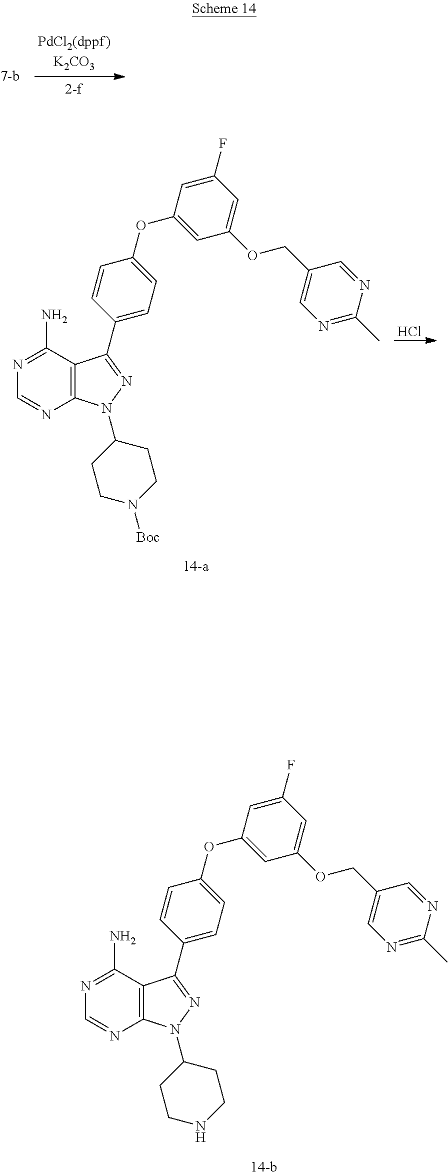 [SMILES: Cc1ncc(COc2cc(F)cc(Oc3ccc(-c4nn(C5CCN(C(=O)OC(C)(C)C)CC5)c5ncnc(N)c45)cc3)c2)cn1 ; Cl ; Cc1ncc(COc2cc(F)cc(Oc3ccc(-c4nn(C5CCNCC5)c5ncnc(N)c45)cc3)c2)cn1]

Step 1:

Intermediate 14-a

To a degassed solution of intermediate 7-b (370 mg, 0.8 mmol), intermediate 2-f (400 mg, 0.9 mmol) and potassium carbonate (345 mg, 2.5 mmol) in DME (4.5 ml) and water (1.1 ml) was added PdCl₂(dppf) (₆₁ mg, 0.08 mmol) and the reaction was heated in a pressure vessel at 105° C. overnight and then cooled to room temperature. Ethyl acetate was added and the reaction was filtered over celite. A saturated aqueous solution of ammonium chloride was added to the filtrate, the organic layer was separated, washed with brine, dried over MgSO₄, filtered and concentrated under reduced pressure. Purification by silica gel chromatography provided intermediate 14-a as a white foam.

Step 2:

Intermediate 14-b

To a solution of intermediate 14-a (522 mg, 0.8 mmol) in 1,4-dioxane (5 ml) and methanol (1 ml) cooled to 0° C. was added a solution of 4N HCl in 1,4-dioxane (4.2 ml, 16.7 mmol). After the addition was completed the reaction was stirred for 1 hour at 0° C. Volatiles were removed under reduced pressure. Diethyl ether was added, a precipitate formed and was collected by filtration to provide intermediate 14-b.3HCl as a white solid.

Synthesis of Intermediate 15-b:

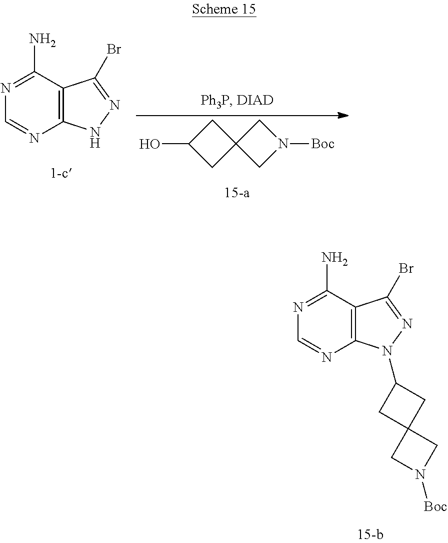 [SMILES: Nc1ncnc2[nH]nc(Br)c12 ; CC(C)(C)OC(=O)N1CC2(CC(O)C2)C1 ; CC(C)(C)OC(=O)N1CC2(CC(n3nc(Br)c4c(N)ncnc43)C2)C1]

To a solution of intermediate 1-c′ (300 mg, 1.4 mmol), intermediate 29-a (344 mg, 1.6 mmol) and triphenylphosphine (404 mg, 1.5 mmol) in THF cooled to 0° C. was added DIAD (300 μl, 1.5 mmol) dropwise. After the addition was completed, the reaction was stirred at room temperature overnight. A saturated aqueous solution of ammonium chloride and ethyl acetate were added, the organic layer was separated, washed with brine, dried over MgSO₄, filtered and concentrated under reduced pressure. Purification by silica gel chromatography provided intermediate 15-b as a yellow solid.

Synthesis of Intermediate 16-b:

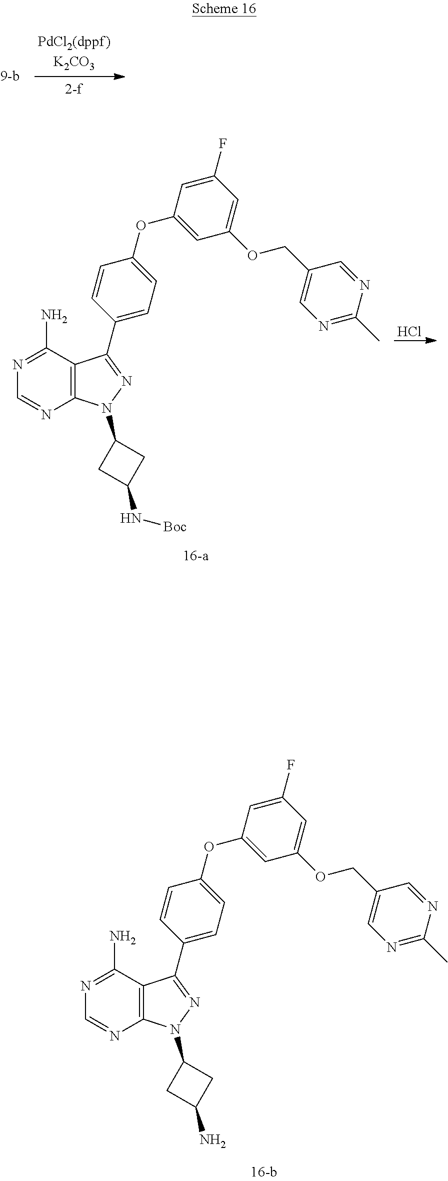 [SMILES: Cc1ncc(COc2cc(F)cc(Oc3ccc(-c4nn([C@H]5C[C@@H](NC(=O)OC(C)(C)C)C5)c5ncnc(N)c45)cc3)c2)cn1 ; Cl ; Cc1ncc(COc2cc(F)cc(Oc3ccc(-c4nn([C@H]5C[C@@H](N)C5)c5ncnc(N)c45)cc3)c2)cn1]

Step 1:

Intermediate 16-a

To a degassed solution of intermediate 9-b (1.0 g, 2.6 mmol), intermediate 2-f (1.25 g, 2.9 mmol) and potassium carbonate (1.1 mg, 7.8 mmol) in DME (13.9 ml) and water (3.5 ml) was added PdCl₂(dppf) (191 mg, 0.26 mmol) and the reaction was heated in a pressure vessel at 105° C. overnight and then cooled to room temperature. Ethyl acetate was added and the reaction was filtered over celite. A saturated aqueous solution of ammonium chloride was added to the filtrate, the organic layer was separated, washed with brine, dried over MgSO₄, filtered and concentrated under reduced pressure. Purification by silica gel chromatography provided intermediate 16-a as a white foam.

Step 2:

Intermediate 16-b

To a solution of intermediate 16-a (1.1 g, 1.8 mmol) in 1,4-dioxane (10 ml) and methanol (1 ml) cooled to 0° C. was added a solution of 4N HCl in 1,4-dioxane (2.2 ml, 71.8 mmol). After the addition was completed the reaction was stirred for 1 hour at room temperature. Diethyl ether was added, a precipitate formed and was collected by filtration to provide intermediate 16-b.3HCl as a yellow solid.

Compounds 1, 7, 10, 14, 15, 16, 17, 19, 20 were prepared as shown in Schemes 17 to 25.

Synthesis of Compound 1:

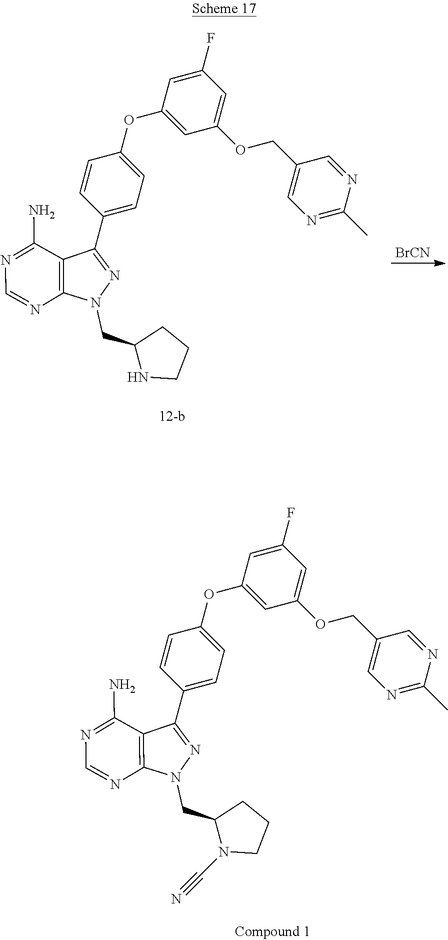

To a solution of intermediate 12-b (100 mg, 0.16 mmol) in dicloromethane (1.6 ml) were sequentially added TEA (219 μl, 1.6 mmol) and cyanic bromide (20 mg, 0.19 mmol) and the reaction was stirred for 2 hours at room temperature. Volatiles were removed under reduced pressure. Purification by silica gel chromatography provided Compound 1 as a beige solid.

Compound 8 and Compound 33 was prepared in a similar manner to Compound 1 starting from intermediate 10-b and 14-b respectively.

Synthesis of Compound 14:

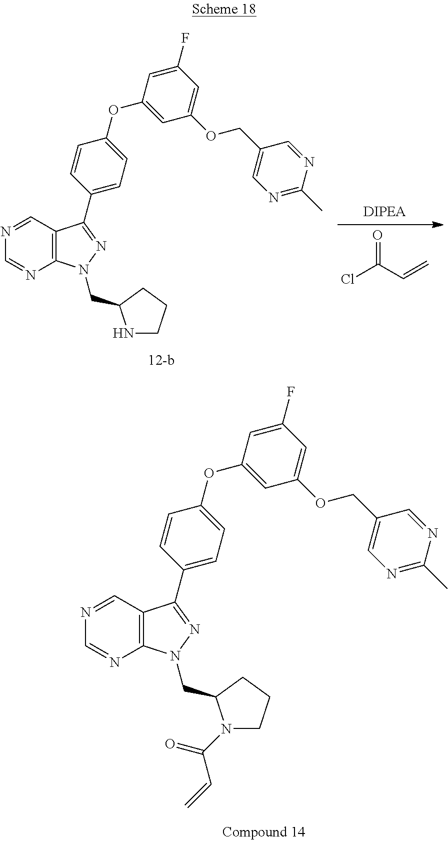

To a solution of intermediate 12-b (100 mg, 0.16 mmol) in NMP (1.6 ml) were sequentially added DIPEA (137 μl, 0.78 mmol) and acryloyl chloride (17 mg, 0.19 mmol) and the reaction was stirred for 2 hours at room temperature. A saturated aqueous solution of ammonium chloride and dichloromethane were added, the organic layer was separated, washed with brine, dried over MgSO₄, filtered and concentrated under reduced pressure. Purification by silica gel chromatography provided Compound 14 as a white solid.

Compounds 12, 22, 23, 25, 27, 31 and 34 were prepared in a similar manner to Compound 14 starting from intermediate 10-b, 11-b, 13-b, 14-b, 16-b, 28-b.3HCl and 29b3TFA respectively.

Synthesis of Compound 16:

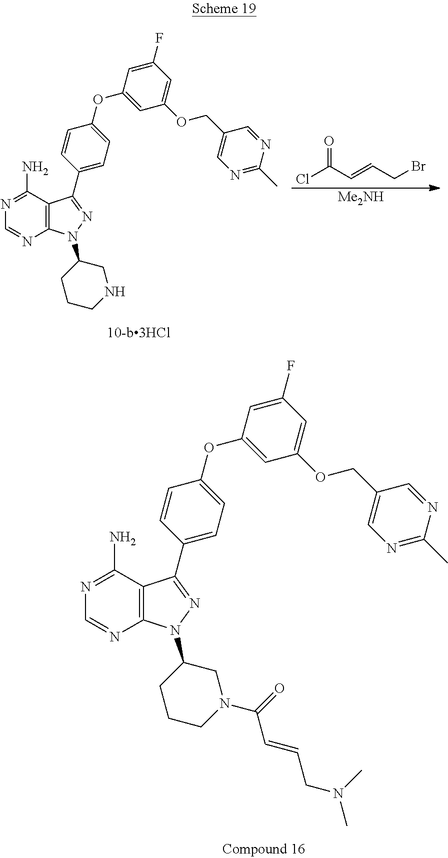

To a solution of intermediate 10-b3HCl (43 mg, 0.26 mmol) in NMP (2.0 ml) cooled to 0° C. were sequentially added DIPEA (206 μl, 0.78 mmol) and a solution of (E)-4-bromobut-2-enoyl chloride in dichloromethane and the reaction was stirred for 1 hour. A 1.0 M solution of dimethylamine (2.3 ml, 2.3 mmol) was added and the mixture was then stirred at room temperature overnight. Volatiles were removed under reduced pressure and the residue was absorbed on silica gel. Purification by silica gel chromatography provided Compound 16 as a white solid.

Preparation of (E)-4-bromobut-2-enoyl chloride:

To a solution of (E)-4-bromobut-2-enoic acid (43 mg, 0.26 mmol) in dichloromethane was added oxalyl chloride (41 μl, 0.47 mmol) and DMF (183 μl, 2.4 mmol) and the solution was stirred at room temperature for 1 hour. Volatiles were removed under reduced pressure and the residue was dissolved in dichloromethane.

Compound 9 was prepared in a similar manner to Compound 16 starting from intermediate 12-b.

Synthesis of Compound 20:

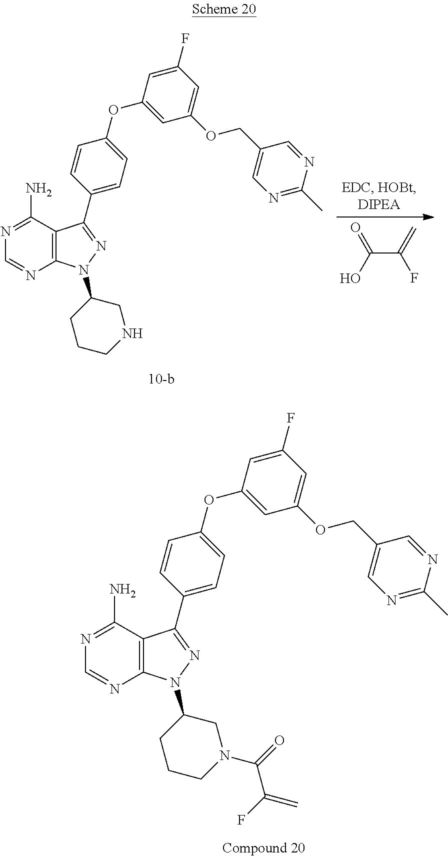

To a solution of intermediate 10-b (100 mg, 0.19 mmol) in DMF (2 ml) were sequentially added DIPEA (165 μl, 0.95 mmol), EDC (55 mg, 0.28 mmol), HOBt (44 mg, 0.28 mmol) and 2-fluoroacrylic acid (21 mg, 0.23 mmol) and the reaction was stirred overnight at room temperature. A saturated aqueous solution of ammonium chloride and ethyl acetate were added, the organic layer was separated, washed with brine, dried over MgSO₄, filtered and concentrated under reduced pressure. Purification by silica gel chromatography provided Compound 20 as a white solid.

Compounds 6 and 18 were prepared in a similar manner to Compound 20 starting from intermediate 12-b and 11-b respectively.

Synthesis of Compound 19:

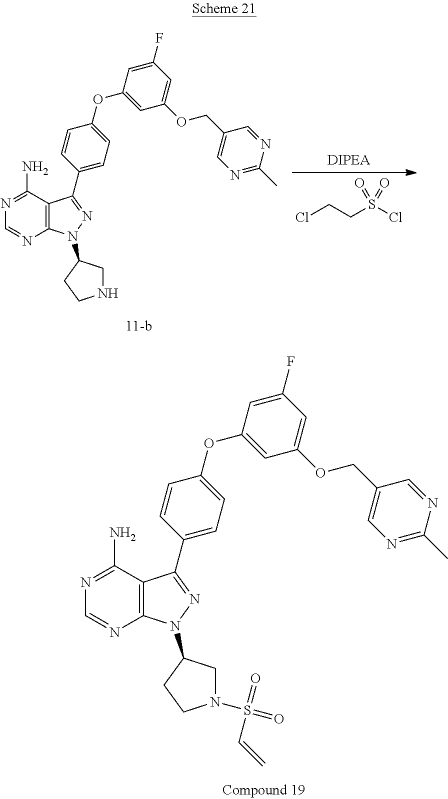 [SMILES: Cc1ncc(COc2cc(F)cc(Oc3ccc(-c4nn([C@@H]5CCNC5)c5ncnc(N)c45)cc3)c2)cn1 ; O=S(=O)(Cl)CCCl ; C=CS(=O)(=O)N1CC[C@@H](n2nc(-c3ccc(Oc4cc(F)cc(OCc5cnc(C)nc5)c4)cc3)c3c(N)ncnc32)C1]

To a solution of intermediate 11-b (100 mg, 0.19 mmol) in dichloromethane (2 ml) were sequentially added DIPEA (170 μl, 0.97 mmol) and 2-cloroethanesulfonyl chloride (23 μl, 0.21 mmol) at 0° C. and the reaction was then stirred overnight at room temperature. A saturated aqueous solution of ammonium chloride and dichloromethane were added, the organic layer was separated, washed with brine, dried over MgSO₄, filtered and concentrated under reduced pressure. Purification by silica gel chromatography provided Compound 19 as a white solid.

Compound 21 was prepared in a similar manner to Compound 19 starting from intermediate 10-b.

Synthesis of Compound 17:

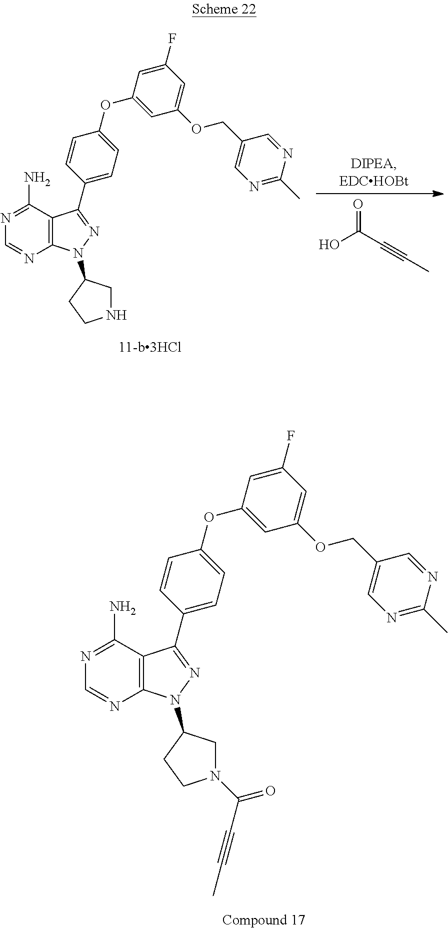

To a solution of intermediate 11-b3HCl (500 mg, 0.8 mmol) in DMF (10 ml) were sequentially added but-2-ynoic acid (86 mg, 1.0 mmol), EDC (246 mg, 1.3 mmol), HOBt (196 mg, 1.3 mmol) and DIPEA (744 μl, 4.3 mmol) and the reaction was then stirred at room temperature for 2 hours. A saturated aqueous solution of ammonium chloride and ethyl acetate were added, the organic layer was separated, washed with brine, dried over MgSO₄, filtered and concentrated under reduced pressure. Purification by silica gel chromatography provided Compound 17 as a white solid.

Compounds 5, 11, 24, 26, 28 and 32 were prepared in a similar manner to Compound 17 starting from intermediate 12-b, 10-b, 13-b, 14-b, 16-b, and 28-b.3HCl respectively.

Synthesis of Compound 10:

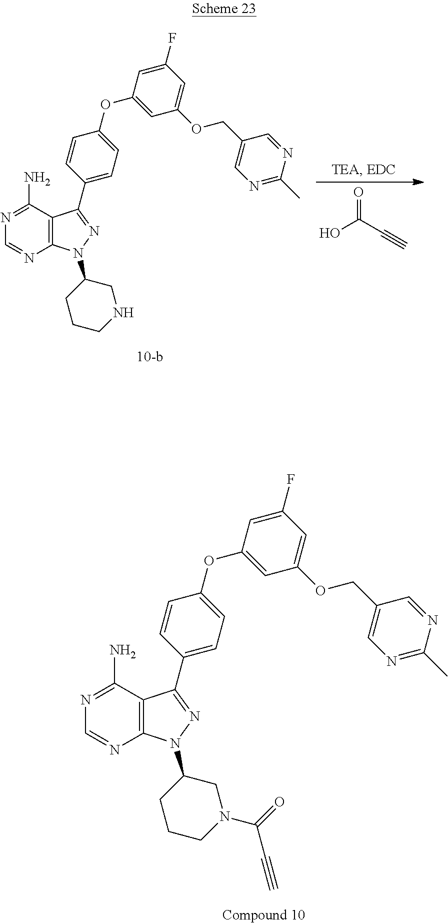

To a solution of intermediate 10-b (65 mg, 0.1 mmol) in dichloromethane (1.0 ml) were sequentially added TEA (142 μl, 1.0 mmol), EDC (29 mg, 0.15 mmol) and propiolic acid (10.7 mg, 0.15 mmol) and the reaction was then stirred at room temperature overnight. Volatiles were removed under reduced pressure and the residue was absorbed on silica gel. Purification by silica gel chromatography provided Compound 10 as a white solid.

Compound 13 was prepared in a similar manner to Compound 10 starting from intermediate 12-b.

Synthesis of Compound 15:

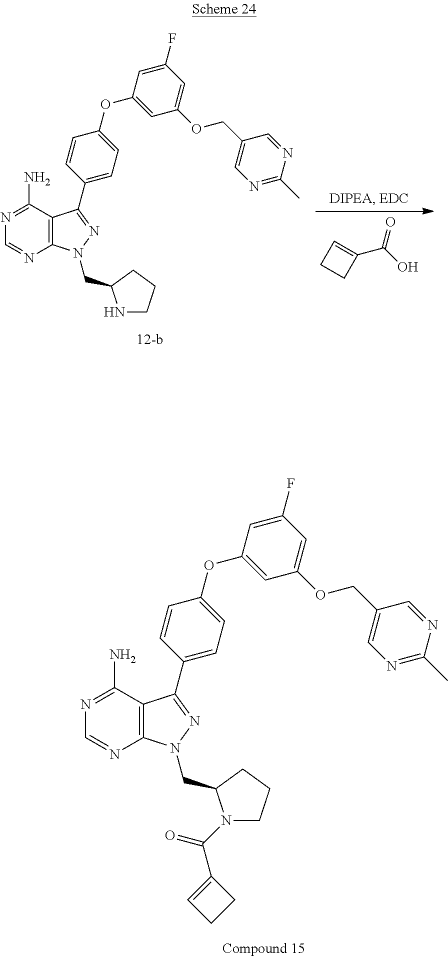 [SMILES: Cc1ncc(COc2cc(F)cc(Oc3ccc(-c4nn(C[C@H]5CCCN5)c5ncnc(N)c45)cc3)c2)cn1 ; O=C(O)C1=CCC1 ; Cc1ncc(COc2cc(F)cc(Oc3ccc(-c4nn(C[C@H]5CCCN5C(=O)C5=CCC5)c5ncnc(N)c45)cc3)c2)cn1]

To a solution of intermediate 12-b (80 mg, 0.12 mmol) in dichloromethane (1.3 ml) were sequentially added DIPEA (109 μl, 0.6 mmol), EDC (24 mg, 0.12 mmol) and cyclobut-1-enecarboxylic acid (19 mg, 0.18 mmol) and the reaction was then stirred at room temperature overnight. Brine and dichloromethane were added; the organic layer was separated, dried over MgSO₄, filtered and concentrated under reduced pressure. Purification by silica gel chromatography provided Compound 15 as a white solid.

Compounds 2 and 3 were prepared in a similar manner to Compound 15 starting from intermediate 12-b and cyclopent-1-enecarboxylic acid, cyclopent-1-enecarboxylic acid respectively.

Synthesis of Compound 7:

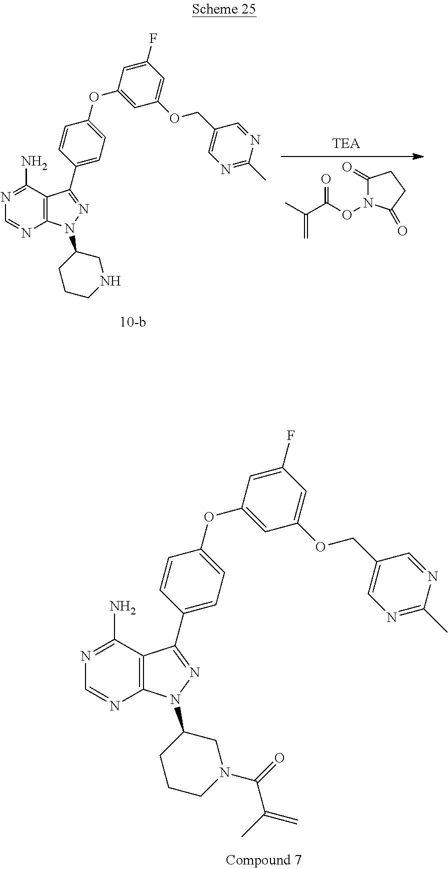

To a solution of intermediate 10-b (80 mg, 0.12 mmol) in DMF (1.3 ml) were sequentially added TEA (219 μl, 1.6 mmol) and 2,5-dioxopyrrolidin-1-yl methacrylate (35 mg, 0.18 mmol) and the reaction was then stirred at room temperature overnight. Brine and ethyl acetate were added; the organic layer was separated, dried over MgSO₄, filtered and concentrated under reduced pressure. Purification by silica gel chromatography provided Compound 7 as a beige solid.

Compound 4 was prepared in a similar manner to Compound 7 starting from intermediate 12-b.

Intermediates 26-c, 27-a, 28-b and 29-b were prepared as shown in Schemes 26 to 29.

Synthesis of Intermediates 26-c

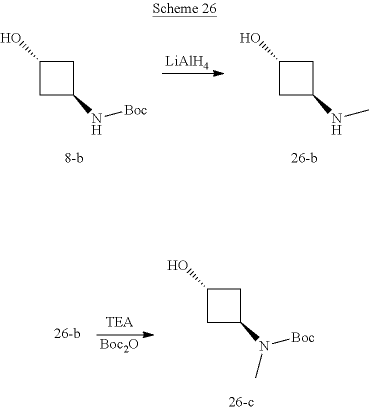

Step 2:

Intermediate 26-b

To a solution of intermediate 8-b (2.0 g, 10.7 mmol) in anhydrous THF (53 ml) cooled to 0° C. was slowly added a 1.0 M solution of LiAlH₄ in THF (32.0 ml, 31.0 mmol). After the addition was completed, the reaction was warmed to room temperature, stirred at 65° C. for 2 hours and then cooled to 0° C. 15% aqueous NaOH was then added and after stirring for 15 minutes the reaction was filtered. The filtrate was concentrated under reduced pressure. Purification by silica gel chromatography provided intermediate 26-b as a white solid.

Step 3:

Intermediate 26-c

To a solution of in intermediate 26-b (800 mg, 7.9 mmol) in ethanol (5.0 ml) were sequentially added TEA (4.9 ml) and Boc₂O (1.7 g, 7.9 mmol) and the reaction was stirred for 4 days at room temperature. Volatiles were removed under reduced pressure, dichloromethane and water were added to the residue, the organic layer was separated, the aqueous layer was extracted twice with dichloromethane, the combined organic extracts were washed with a saturated aqueous solution of NaHCO₃ and brine, dried over MgSO₄, filtered and concentrated under reduced pressure to provide intermediate 26-c as a colorless oil.

Synthesis of Intermediate 27-a:

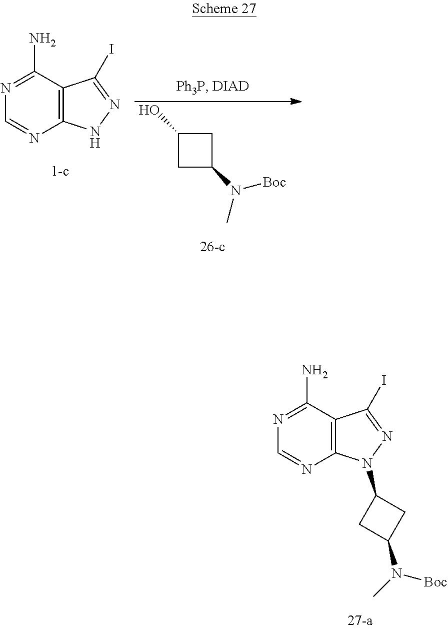

To a solution of intermediate 1-c (259 mg, 1.0 mmol), intermediate 26-c (200 mg, 1.0 mmol) and triphenylphosphine polymer-bound (1.0 g, ˜3 mmol/g triphenyl phosphine loading) in THF cooled to 0° C. was added DIAD (580 μl, 3.0 mmol) dropwise. After the addition was completed, the reaction was stirred at room temperature for 5 minutes, at 60° C. for 2 hours and then cooled to room temperature. The reaction was filtered and the filtrate was adsorbed on silica gel. Purification by silica gel chromatography provided intermediate 27-a as a white solid.

Synthesis of Intermediate 28-b:

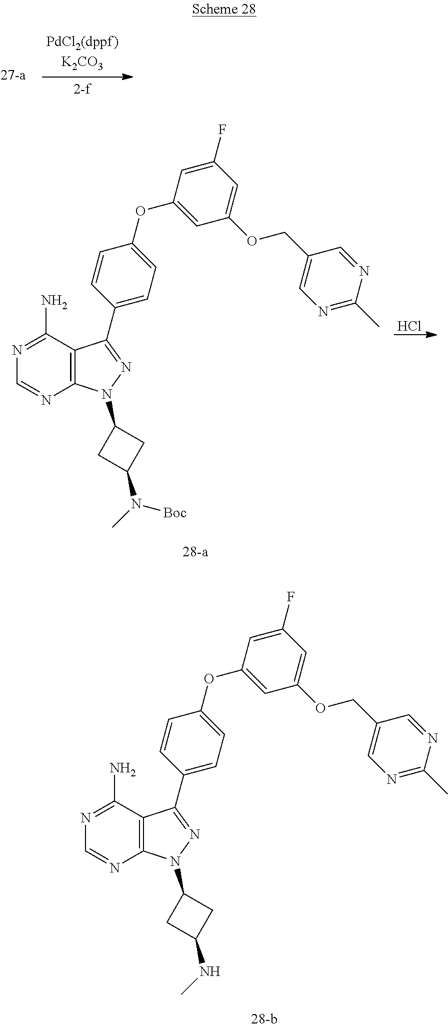 [SMILES: Cc1ncc(COc2cc(F)cc(Oc3ccc(-c4nn([C@H]5C[C@@H](N(C)C(=O)OC(C)(C)C)C5)c5ncnc(N)c45)cc3)c2)cn1 ; Cl ; CN[C@H]1C[C@@H](n2nc(-c3ccc(Oc4cc(F)cc(OCc5cnc(C)nc5)c4)cc3)c3c(N)ncnc32)C1]

Step 1:

Intermediate 28-a

To a degassed solution of intermediate 27-a (150 mg, 0.4 mmol), intermediate 2-f (162 mg, 0.4 mmol) and potassium carbonate (140 mg, 1.0 mmol) in DME (1.8 ml) and water (450 μl) was added PdCl₂ (dppf) (25 mg, 0.03 mmol) and the reaction was heated in a pressure vessel at 105° C. for 2 hours and then cooled to room temperature. Ethyl acetate was added and the reaction was filtered over celite. A saturated aqueous solution of ammonium chloride was added to the filtrate, the organic layer was separated, washed with brine, dried over MgSO₄, filtered and concentrated under reduced pressure. Purification by silica gel chromatography provided intermediate 28-a as a white foam.

Step 2:

Intermediate 28-b

To a solution of intermediate 28-a (150 mg, 0.2 mmol) in 1,4-dioxane (5 ml) and methanol (1 ml) cooled to 0° C. was added a solution of 4N HCl in 1,4-dioxane (1.2 ml, 4.8 mmol). After the addition was completed the reaction was stirred for 1 hour at 0° C. Volatiles were removed under reduced pressure. Diethyl ether was added, a precipitate formed and was collected by filtration to provide intermediate 28-b.3HCl as a white solid.

Synthesis of Intermediate 29-b:

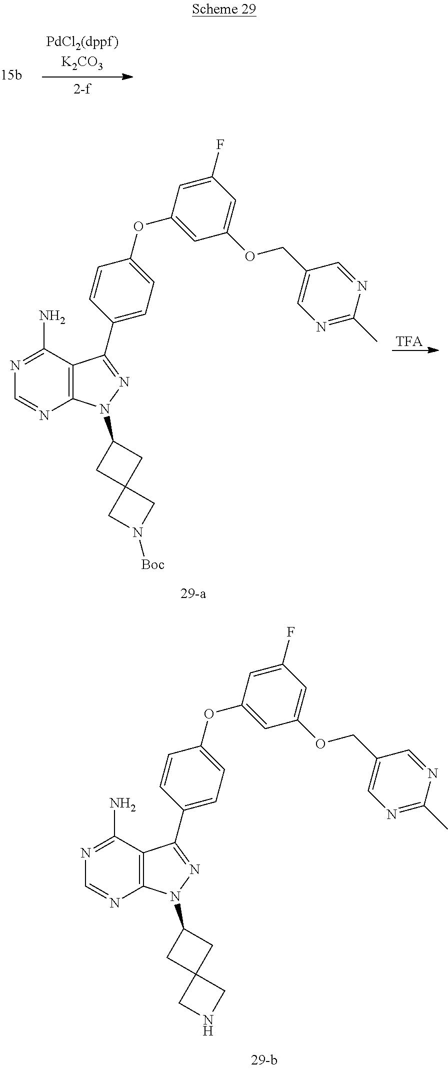

Step 1:

Intermediate 29-a

To a degassed solution of intermediate 15-b (281 mg, 0.6 mmol), intermediate 2-f (281 mg, 0.6 mmol) and potassium carbonate (243 mg, 1.7 mmol) in DME (3.2 ml) and water (780 μl) was added PdCl₂(dppf) (43 mg, 0.06 mmol) and the reaction was heated in a pressure vessel at 105° C. for 2 hours and then cooled to room temperature. Ethyl acetate was added and the reaction was filtered over celite. A saturated aqueous solution of ammonium chloride was added to the filtrate, the organic layer was separated, washed with brine, dried over MgSO₄, filtered and concentrated under reduced pressure. Purification by silica gel chromatography provided intermediate 29-a as a white solid.

Step 2:

Intermediate 29-b

To a solution of intermediate 30-a (350 mg, 0.5 mmol) in dichloromethane (5 ml) was added TFA (42 μl, 0.5 mmol) and the reaction was stiired at room temperature for 1 hour. Volatiles were removed under reduced pressure. Diethyl ether was added to the residue; a precipitate formed and was collected by filtration to provide intermediate 29-b3TFA as a beige solid.

TABLE 1
Example Compounds of Formula I

| Compound | Structure | MS (m/z) |
|---|---|---|
| 1 | 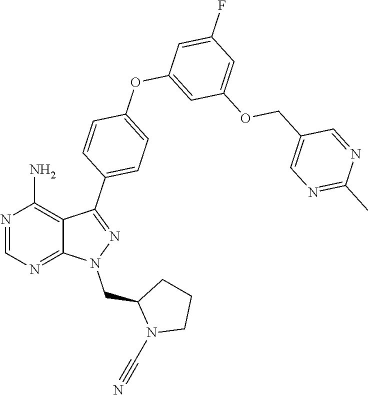 [SMILES: Cc1ncc(COc2cc(F)cc(Oc3ccc(-c4nn(C[C@H]5CCCN5C#N)c5ncnc(N)c45)cc3)c2)cn1] | [M + H]⁺ = 552.3 |
| 2 | 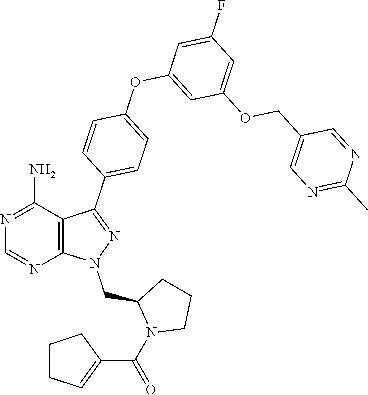 [SMILES: Cc1ncc(COc2cc(F)cc(Oc3ccc(-c4nn(C[C@H]5CCCN5C(=O)C5=CCCC5)c5ncnc(N)c45)cc3)c2)cn1] | [M + H]⁺ = 621.3 |
| 3 | 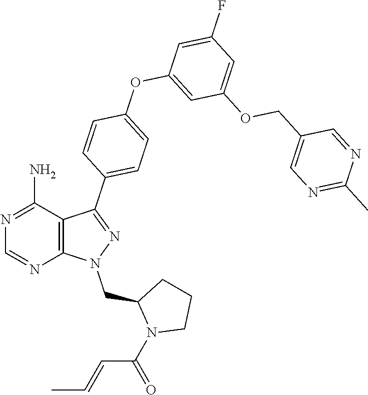 [SMILES: C/C=C/C(=O)N1CCC[C@@H]1Cn1nc(-c2ccc(Oc3cc(F)cc(OCc4cnc(C)nc4)c3)cc2)c2c(N)ncnc21] | [M + H]⁺ = 595.3 |
| 4 | 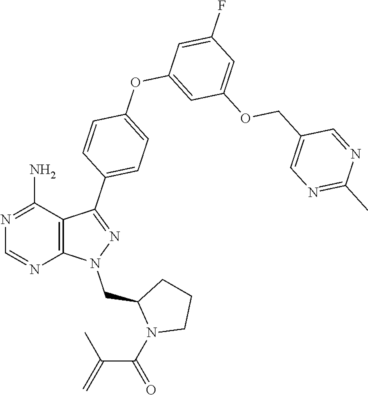 [SMILES: C=C(C)C(=O)N1CCC[C@@H]1Cn1nc(-c2ccc(Oc3cc(F)cc(OCc4cnc(C)nc4)c3)cc2)c2c(N)ncnc21] | [M + H]⁺ = 595.3 |
| 5 | 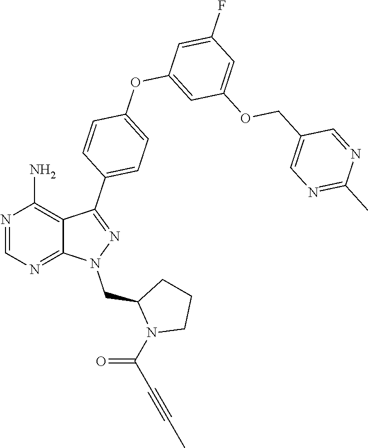 [SMILES: CC#CC(=O)N1CCC[C@@H]1Cn1nc(-c2ccc(Oc3cc(F)cc(OCc4cnc(C)nc4)c3)cc2)c2c(N)ncnc21] | [M + H]⁺ = 593.3 |
| 6 | 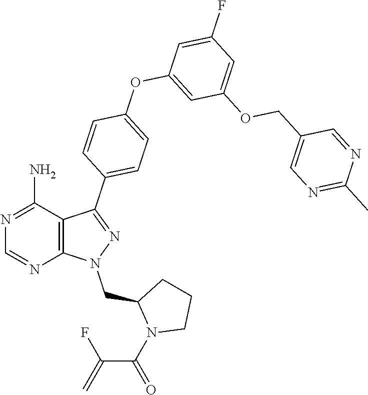 [SMILES: C=C(F)C(=O)N1CCC[C@@H]1Cn1nc(-c2ccc(Oc3cc(F)cc(OCc4cnc(C)nc4)c3)cc2)c2c(N)ncnc21] | [M + H]⁺ = 599.2 |
| 7 | 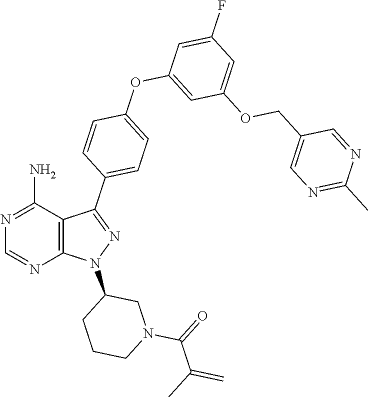 [SMILES: C=C(C)C(=O)N1CCC[C@@H](n2nc(-c3ccc(Oc4cc(F)cc(OCc5cnc(C)nc5)c4)cc3)c3c(N)ncnc32)C1] | [M + H]⁺ = 595.3 |
| 8 | 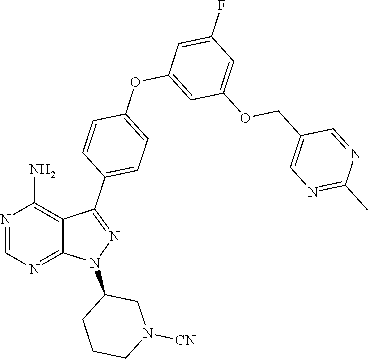 [SMILES: Cc1ncc(COc2cc(F)cc(Oc3ccc(-c4nn([C@@H]5CCCN(C#N)C5)c5ncnc(N)c45)cc3)c2)cn1] | [M + H]⁺ = 552.2 |
| 9 | 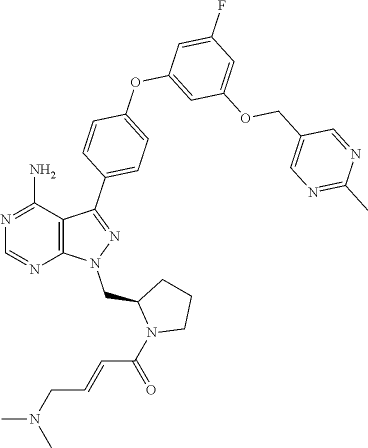 | [M + H]⁺ = 638.2 |
| 10 | 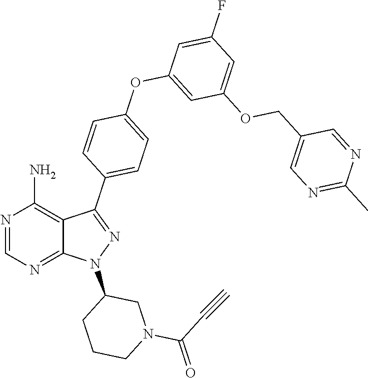 [SMILES: C#CC(=O)N1CCC[C@@H](n2nc(-c3ccc(Oc4cc(F)cc(OCc5cnc(C)nc5)c4)cc3)c3c(N)ncnc32)C1] | [M + H]⁺ = 579.3 |
| 11 | 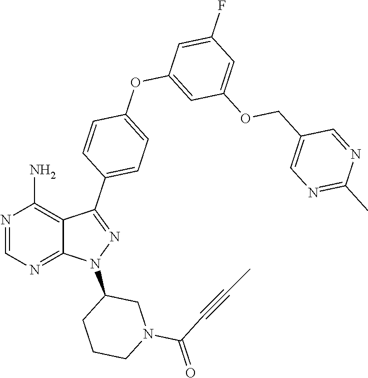 [SMILES: CC#CC(=O)N1CCC[C@@H](n2nc(-c3ccc(Oc4cc(F)cc(OCc5cnc(C)nc5)c4)cc3)c3c(N)ncnc32)C1] | [M + H]⁺ = 593.3 |
| 12 | 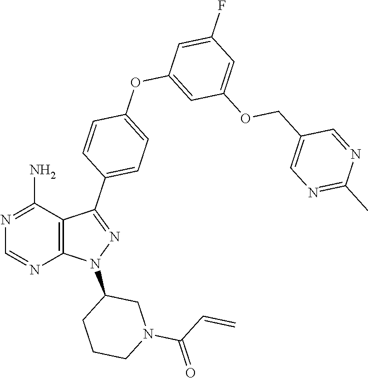 [SMILES: C=CC(=O)N1CCC[C@@H](n2nc(-c3ccc(Oc4cc(F)cc(OCc5cnc(C)nc5)c4)cc3)c3c(N)ncnc32)C1] | [M + H]⁺ = 581.2 |
| 13 | 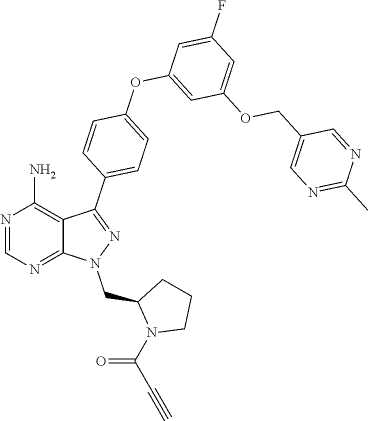 [SMILES: C#CC(=O)N1CCC[C@@H]1Cn1nc(-c2ccc(Oc3cc(F)cc(OCc4cnc(C)nc4)c3)cc2)c2c(N)ncnc21] | [M + H]⁺ = 579.2 |
| 14 | 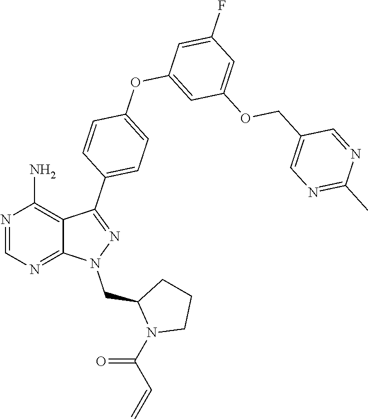 [SMILES: C=CC(=O)N1CCC[C@@H]1Cn1nc(-c2ccc(Oc3cc(F)cc(OCc4cnc(C)nc4)c3)cc2)c2c(N)ncnc21] | [M + H]⁺ = 581.2 |
| 15 | 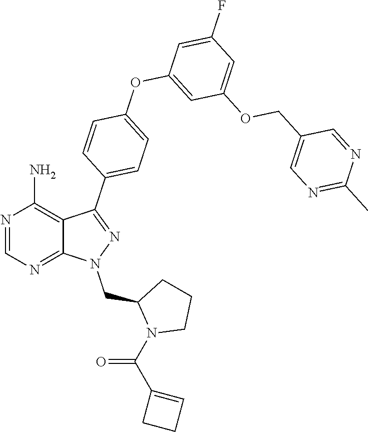 [SMILES: Cc1ncc(COc2cc(F)cc(Oc3ccc(-c4nn(C[C@H]5CCCN5C(=O)C5=CCC5)c5ncnc(N)c45)cc3)c2)cn1] | [M + H]⁺ = 607.2 |
| 16 | 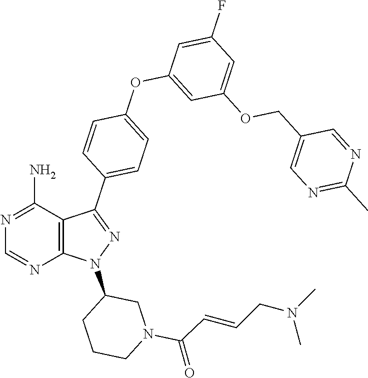 [SMILES: Cc1ncc(COc2cc(F)cc(Oc3ccc(-c4nn([C@@H]5CCCN(C(=O)/C=C/CN(C)C)C5)c5ncnc(N)c45)cc3)c2)cn1] | [M + H]⁺ = 638.2 |
| 17 | 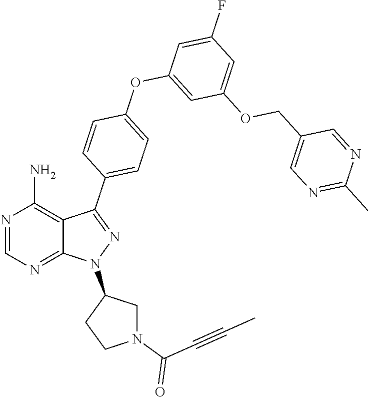 [SMILES: CC#CC(=O)N1CC[C@@H](n2nc(-c3ccc(Oc4cc(F)cc(OCc5cnc(C)nc5)c4)cc3)c3c(N)ncnc32)C1] | [M + H]⁺ = 579.3 |
| 18 | 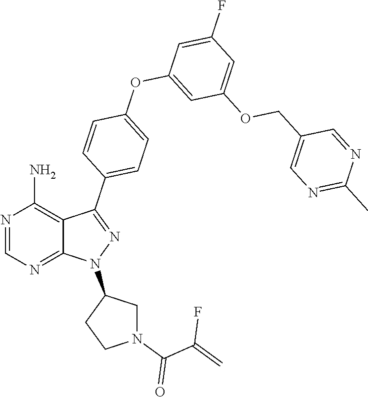 [SMILES: C=C(F)C(=O)N1CC[C@@H](n2nc(-c3ccc(Oc4cc(F)cc(OCc5cnc(C)nc5)c4)cc3)c3c(N)ncnc32)C1] | [M + H]⁺ = 585.2 |
| 19 | 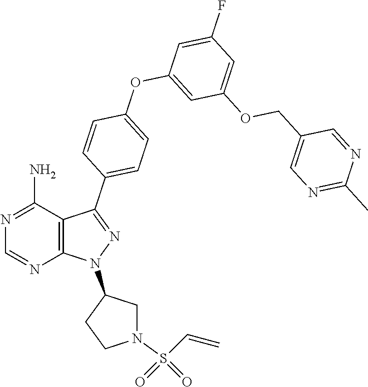 [SMILES: C=CS(=O)(=O)N1CC[C@@H](n2nc(-c3ccc(Oc4cc(F)cc(OCc5cnc(C)nc5)c4)cc3)c3c(N)ncnc32)C1] | [M + H]⁺ = 603.2 |
| 20 | 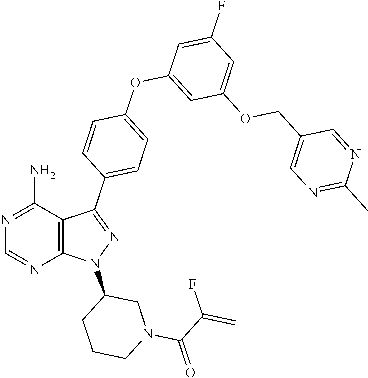 [SMILES: C=C(F)C(=O)N1CCC[C@@H](n2nc(-c3ccc(Oc4cc(F)cc(OCc5cnc(C)nc5)c4)cc3)c3c(N)ncnc32)C1] | [M + H]⁺ = 599.2 |
| 21 | 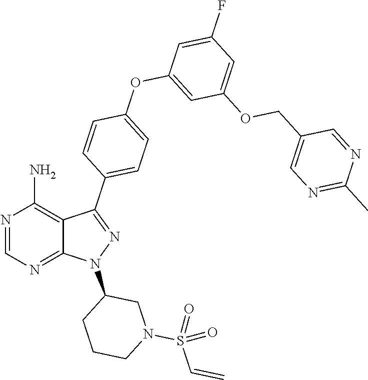 [SMILES: C=CS(=O)(=O)N1CCC[C@@H](n2nc(-c3ccc(Oc4cc(F)cc(OCc5cnc(C)nc5)c4)cc3)c3c(N)ncnc32)C1] | [M + H]⁺ = 617.1 |
| 22 | 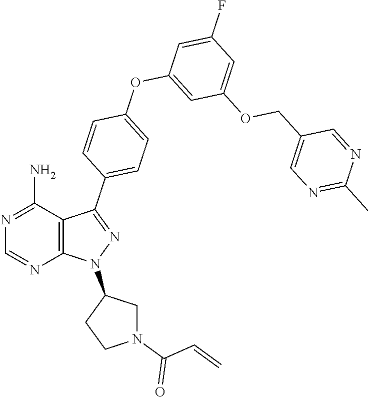 [SMILES: C=CC(=O)N1CC[C@@H](n2nc(-c3ccc(Oc4cc(F)cc(OCc5cnc(C)nc5)c4)cc3)c3c(N)ncnc32)C1] | [M + H]⁺ = 567.3 |
| 23 | 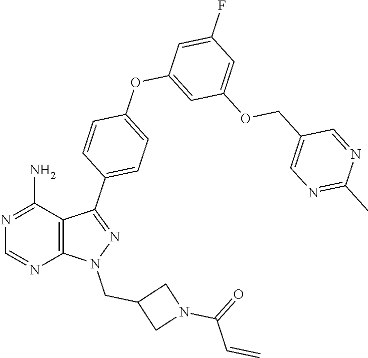 [SMILES: C=CC(=O)N1CC(Cn2nc(-c3ccc(Oc4cc(F)cc(OCc5cnc(C)nc5)c4)cc3)c3c(N)ncnc32)C1] | [M + H]⁺ = 567.3 |
| 3339924 | 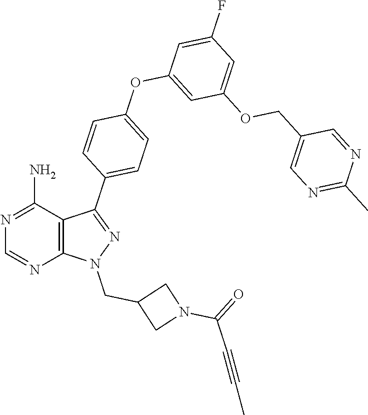 [SMILES: CC#CC(=O)N1CC(Cn2nc(-c3ccc(Oc4cc(F)cc(OCc5cnc(C)nc5)c4)cc3)c3c(N)ncnc32)C1] | [M + H]⁺ = 579.4 |
| 25 | 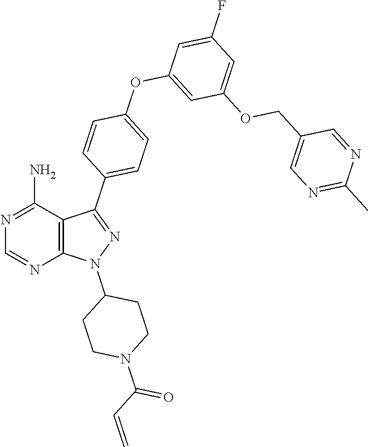 [SMILES: C=CC(=O)N1CCC(n2nc(-c3ccc(Oc4cc(F)cc(OCc5cnc(C)nc5)c4)cc3)c3c(N)ncnc32)CC1] | [M + H]⁺ = 581.3 |
| 26 | 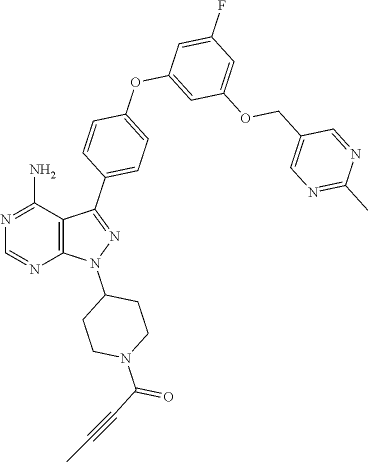 [SMILES: CC#CC(=O)N1CCC(n2nc(-c3ccc(Oc4cc(F)cc(OCc5cnc(C)nc5)c4)cc3)c3c(N)ncnc32)CC1] | [M + H]⁺ = 593.3 |
| 27 | 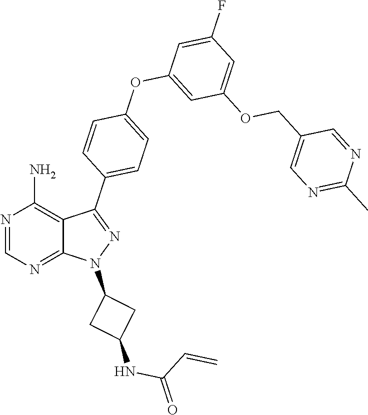 [SMILES: C=CC(=O)N[C@H]1C[C@@H](n2nc(-c3ccc(Oc4cc(F)cc(OCc5cnc(C)nc5)c4)cc3)c3c(N)ncnc32)C1] | [M + H]⁺ = 566.3 |
| 28 | 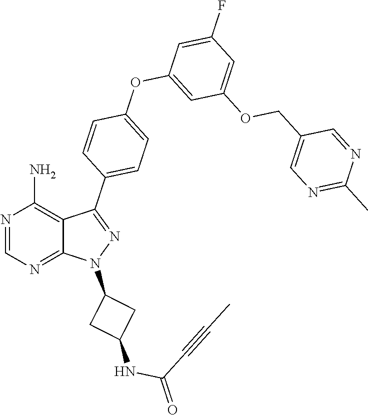 [SMILES: CC#CC(=O)N[C@H]1C[C@@H](n2nc(-c3ccc(Oc4cc(F)cc(OCc5cnc(C)nc5)c4)cc3)c3c(N)ncnc32)C1] | [M + H]⁺ = 579.3 |
| 29 | 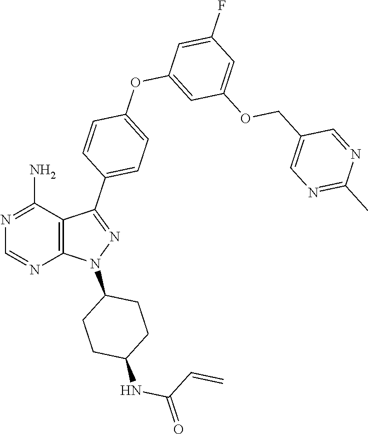 [SMILES: C=CC(=O)N[C@H]1CC[C@@H](n2nc(-c3ccc(Oc4cc(F)cc(OCc5cnc(C)nc5)c4)cc3)c3c(N)ncnc32)CC1] | [M + H]⁺ = 595.3 |
| 30 | 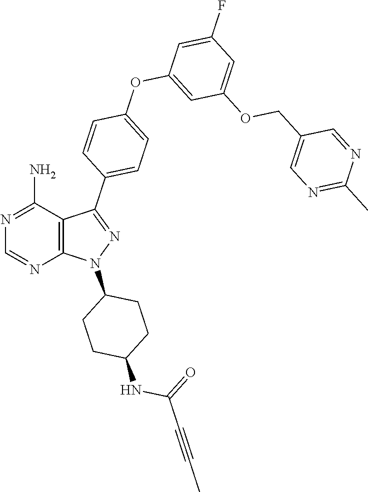 [SMILES: CC#CC(=O)N[C@H]1CC[C@@H](n2nc(-c3ccc(Oc4cc(F)cc(OCc5cnc(C)nc5)c4)cc3)c3c(N)ncnc32)CC1] | [M + H]⁺ = 607.3 |
| 31 | 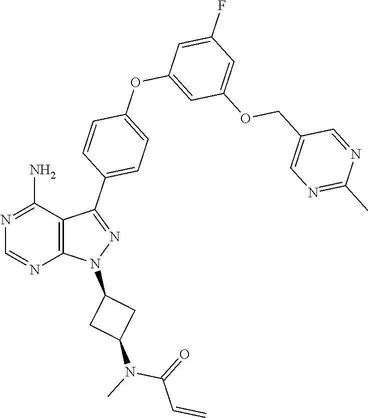 [SMILES: C=CC(=O)N(C)[C@H]1C[C@@H](n2nc(-c3ccc(Oc4cc(F)cc(OCc5cnc(C)nc5)c4)cc3)c3c(N)ncnc32)C1] | [M + H]⁺ = 581.4 |
| 32 | 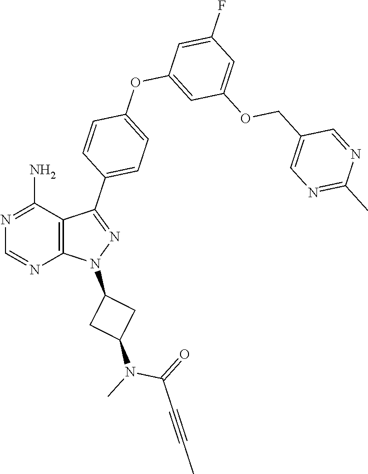 [SMILES: CC#CC(=O)N(C)[C@H]1C[C@@H](n2nc(-c3ccc(Oc4cc(F)cc(OCc5cnc(C)nc5)c4)cc3)c3c(N)ncnc32)C1] | [M + H]⁺ = 593.3 |
| 33 | 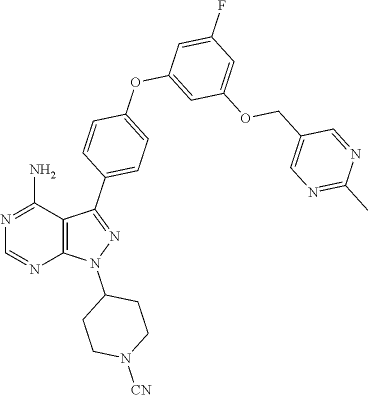 [SMILES: Cc1ncc(COc2cc(F)cc(Oc3ccc(-c4nn(C5CCN(C#N)CC5)c5ncnc(N)c45)cc3)c2)cn1] | [M + H]⁺ = 552.2 |
| 34 | 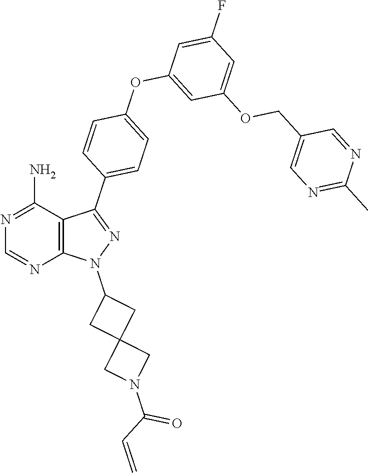 [SMILES: C=CC(=O)N1CC2(CC(n3nc(-c4ccc(Oc5cc(F)cc(OCc6cnc(C)nc6)c5)cc4)c4c(N)ncnc43)C2)C1] | [M + H]⁺ = 593.4 |
| 35 | 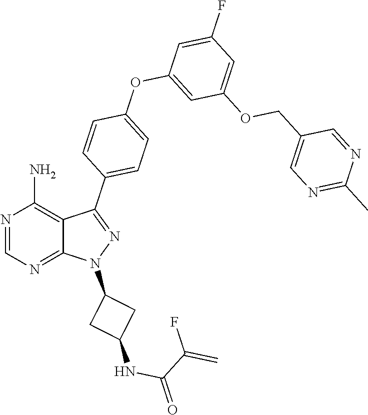 [SMILES: C=C(F)C(=O)N[C@H]1C[C@@H](n2nc(-c3ccc(Oc4cc(F)cc(OCc5cnc(C)nc5)c4)cc3)c3c(N)ncnc32)C1] | [M + H]⁺ = 585.2 |

The following section describes general synthetic method(s) which may be useful in the preparation of compounds of the instant invention and are not intended to be limiting.

Compounds of the present invention, where A is

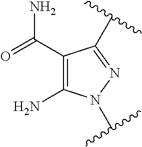 [SMILES: *c1nn(*)c(N)c1C(N)=O]

are prepared according to the following procedure:

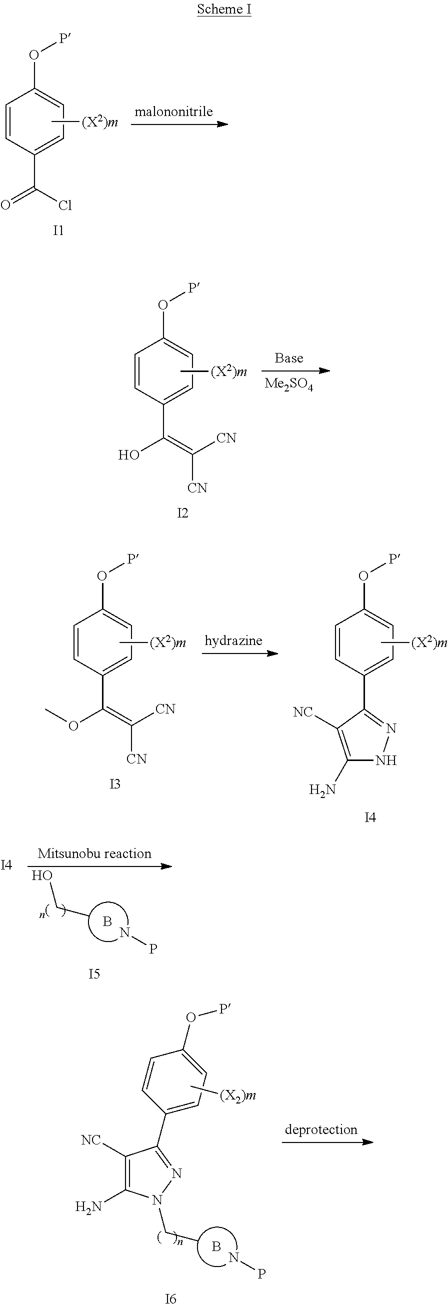

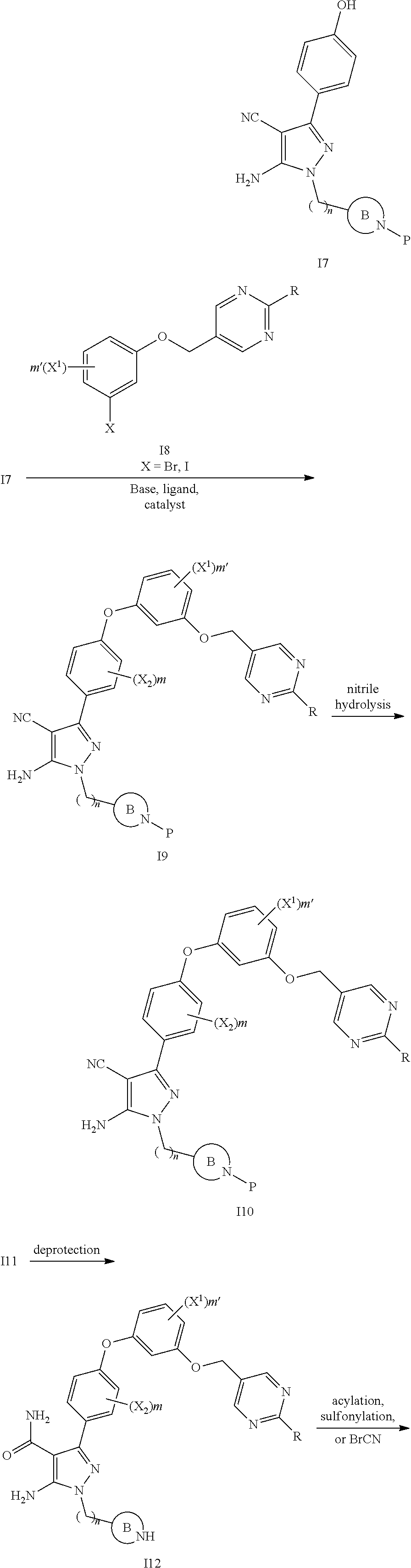

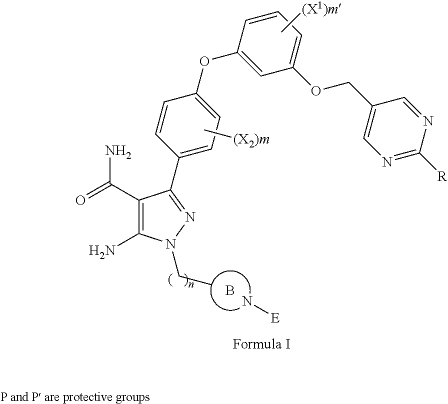

Compounds of the present invention, where A is

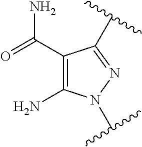 [SMILES: *c1nn(*)c(N)c1C(N)=O]

and

L-E is

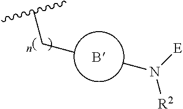

are prepared in a similar manner described above by substituting

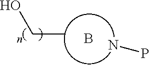

with

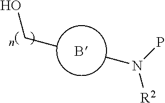

where rings B and B′, n, R, R² and E are as defined above.

Representative examples are listed below:

| Examples | |
|---|---|
| No. | Structure |
| 36 | 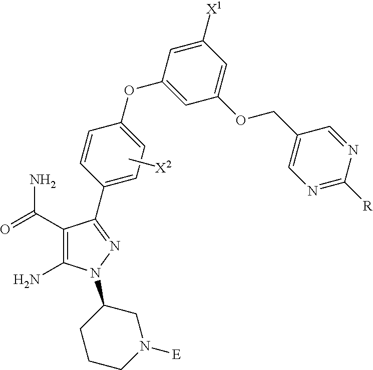 |
| 37 | 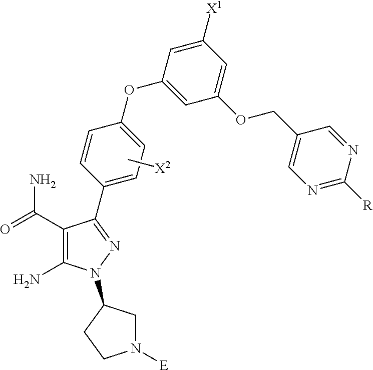 |
| 38 | 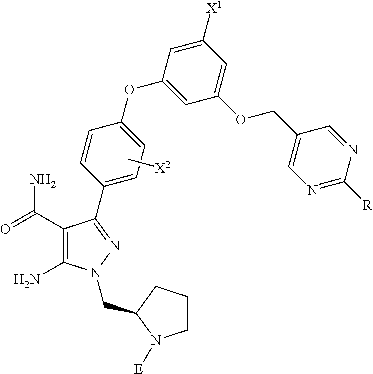 |
| 39 | 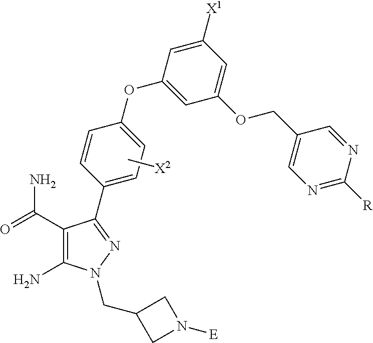 |
| 40 | 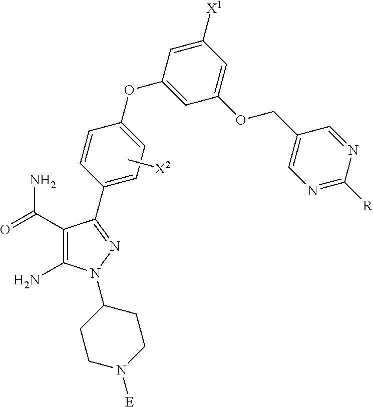 |
| 41 | 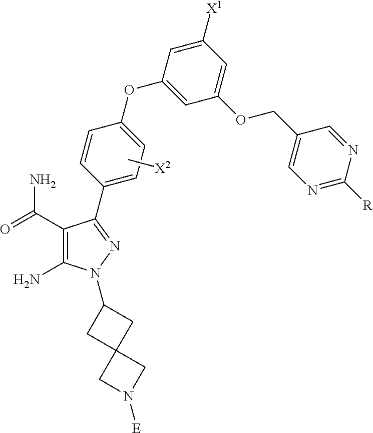 |
| 42 | 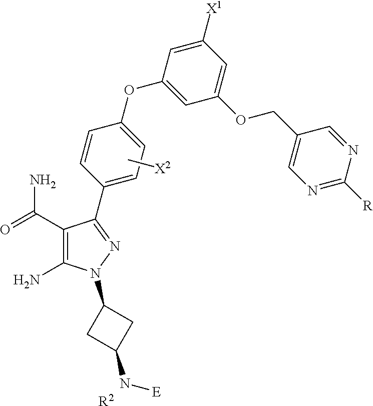 |
| 43 | 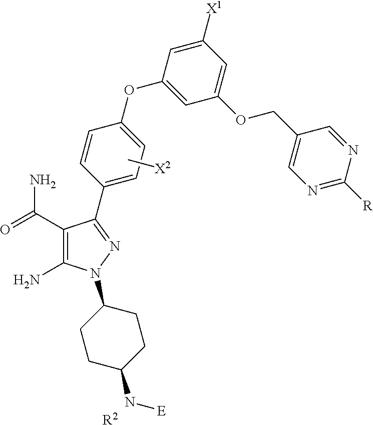 |

wherein:
X¹ and X² are independently selected from hydrogen and fluorine;
R² is selected from hydrogen and methyl;
E is selected from

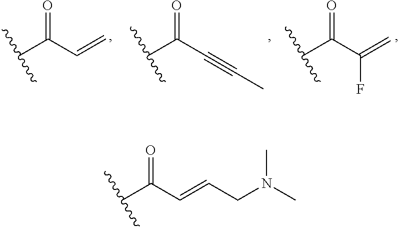

### and —CN, and

R is selected from hydrogen and methyl.

Compounds of the instant invention, where A is

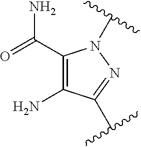 [SMILES: *c1nn(*)c(C(N)=O)c1N]

are prepared according to the following procedure:

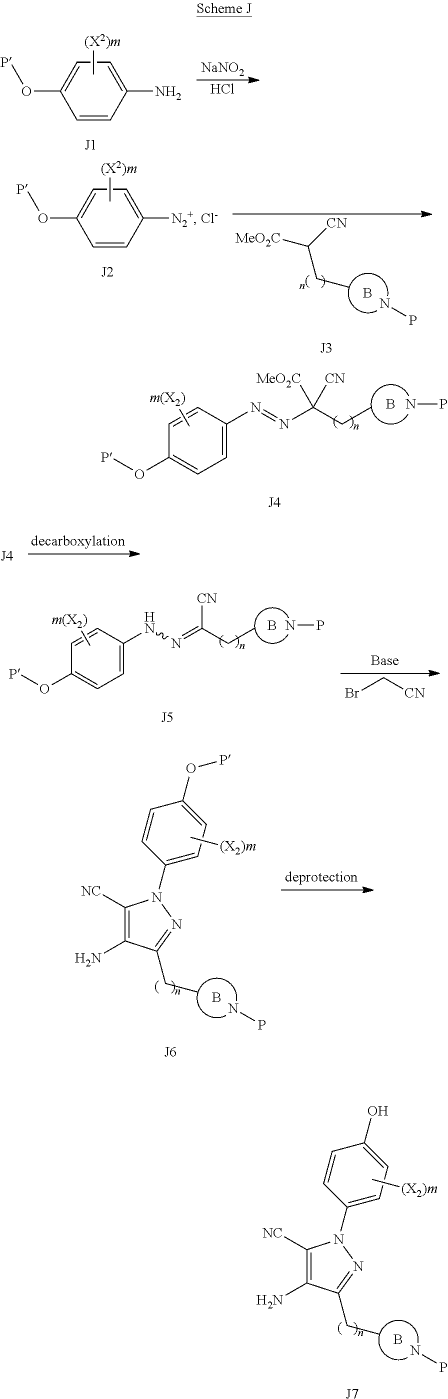

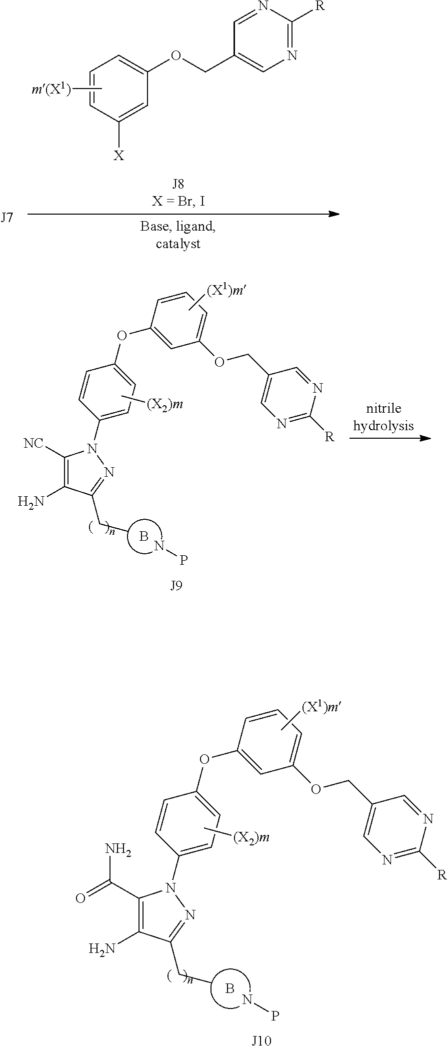

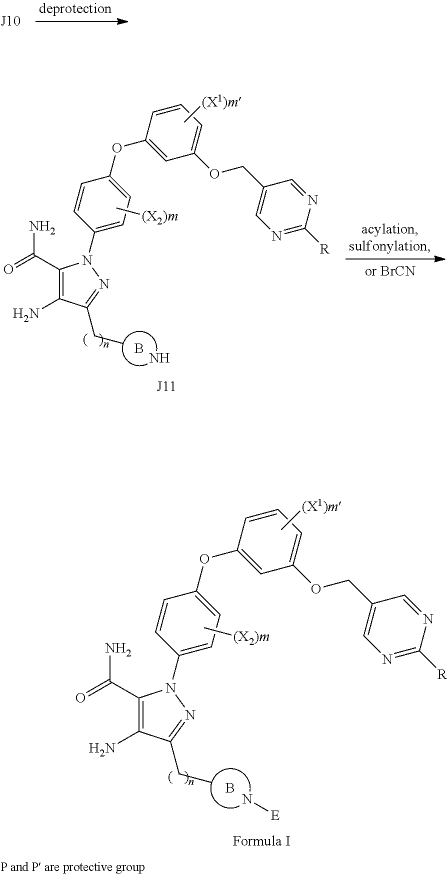

Compounds of the instant invention, where A is

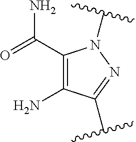 [SMILES: *c1nn(*)c(C(N)=O)c1N]

and

L-E is

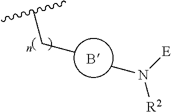

are prepared in a similar manner by substituting

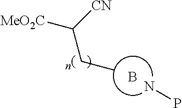

with

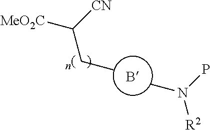

where rings B and B′, n, R, R² and E are as defined above.

Representative examples are listed below:

| Examples | |
|---|---|
| No. | Structure |
| 44 | 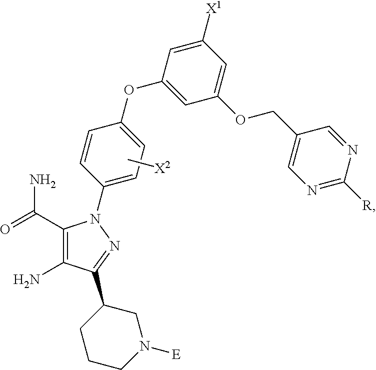 |
| 45 | 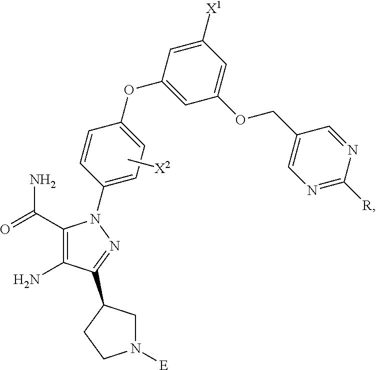 |
| 46 | 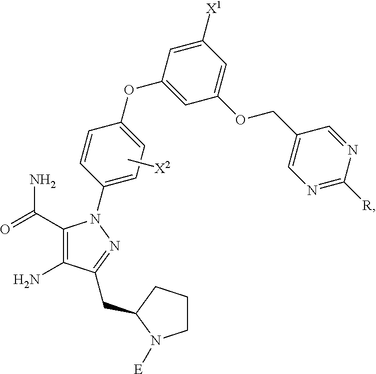 |
| 47 | 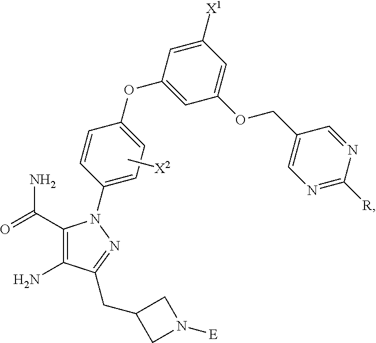 |
| 48 | 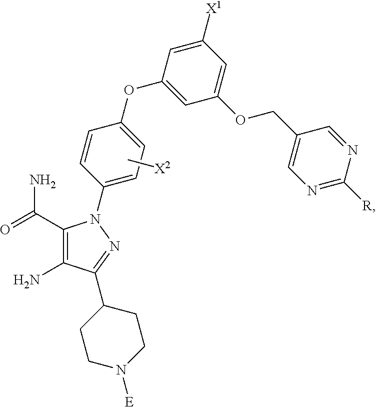 |
| 49 | 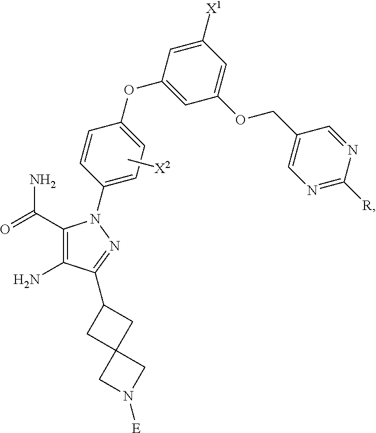 |
| 50 | 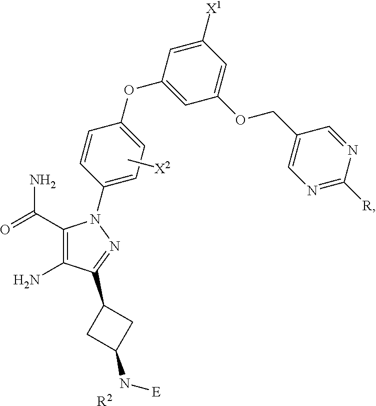 |
| 51 | 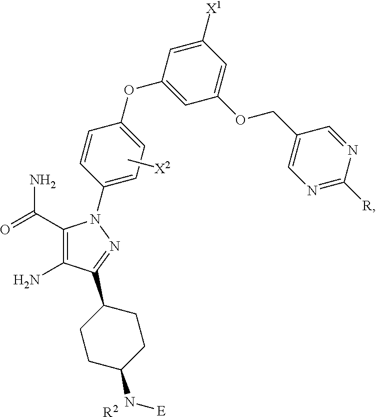 |

wherein:
X¹ and X² are independently selected from hydrogen and fluorine;
R² is selected from hydrogen and methyl;
E is selected from

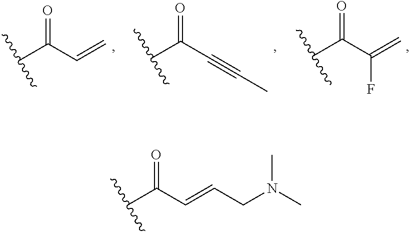

### and —CN, and

R is selected from hydrogen and methyl.

Compounds of the instant invention, where A is

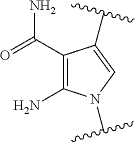 [SMILES: *c1cn(*)c(N)c1C(N)=O]

can be prepared according to the following procedure:

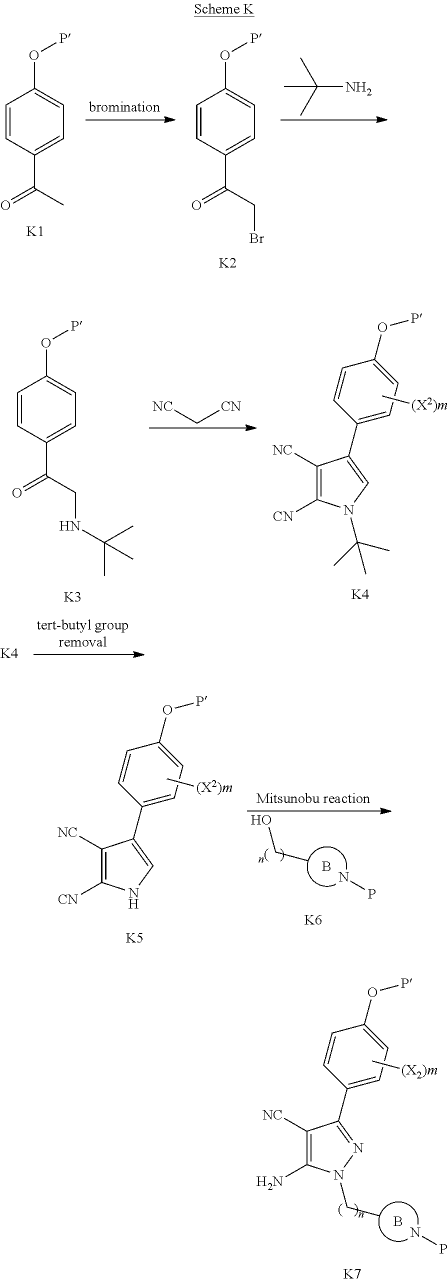

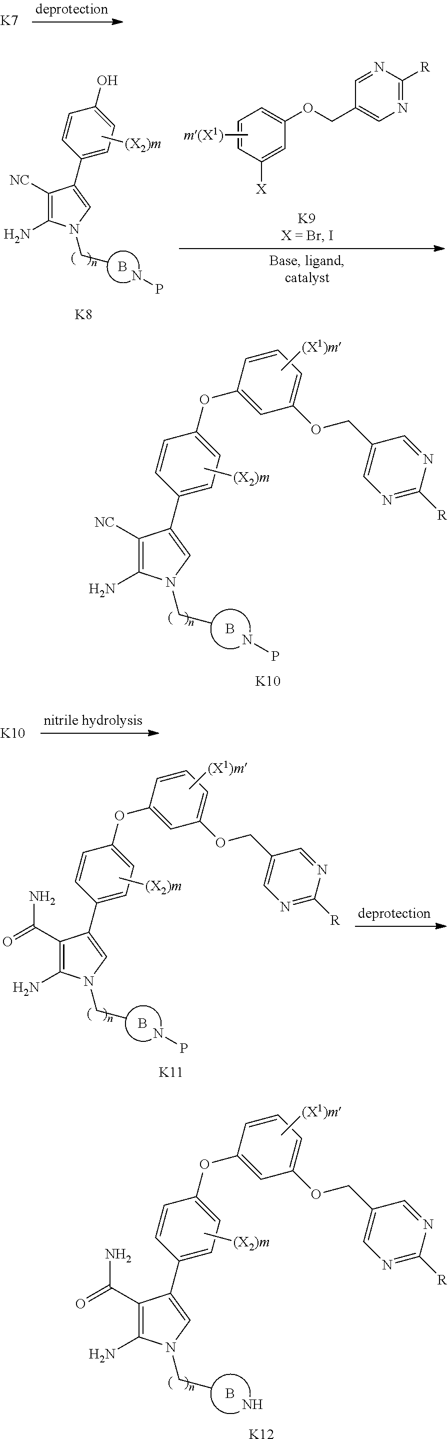

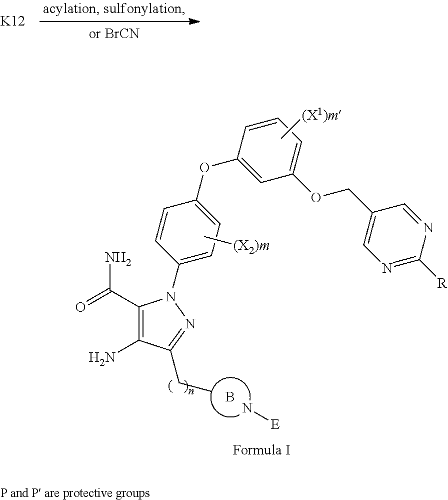

Compounds of the instant invention, where A is

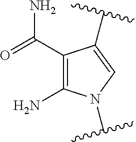 [SMILES: *c1cn(*)c(N)c1C(N)=O]

and L-E is

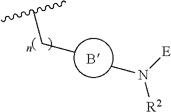

are prepared in a similar manner by substituting

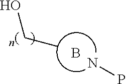

with

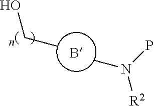

where rings B and B′, n, R, R² and E are as defined above.

Representative examples are listed below:

| Examples | |
|---|---|
| No | Structure |
| 52 | 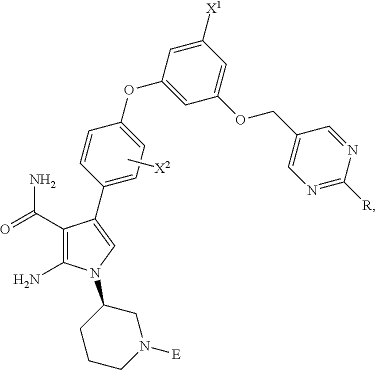 |
| 53 | 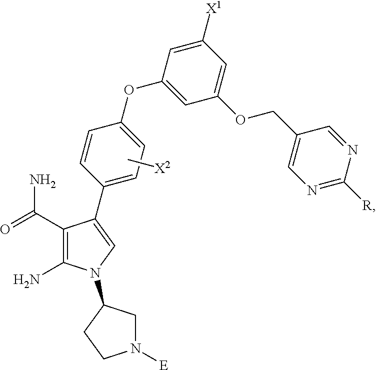 |
| 54 | 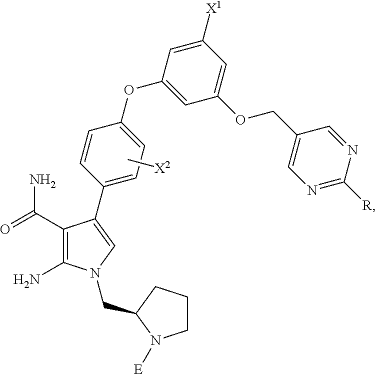 |
| 55 | 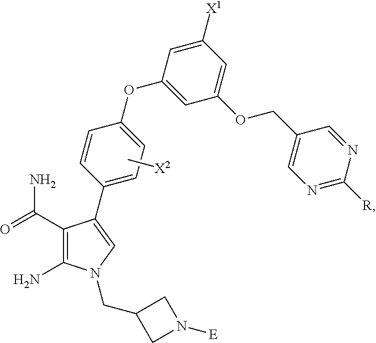 |
| 56 | 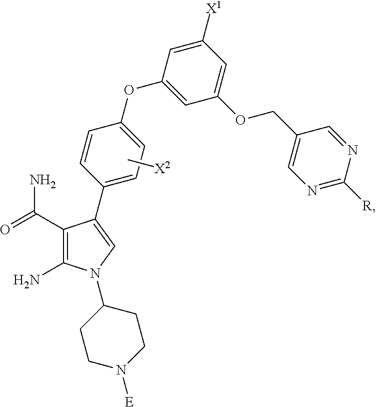 |
| 57 | 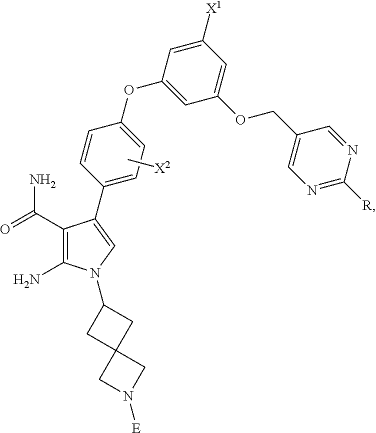 |
| 58 | 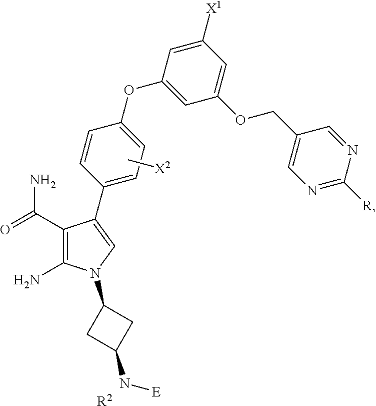 |
| 53 | 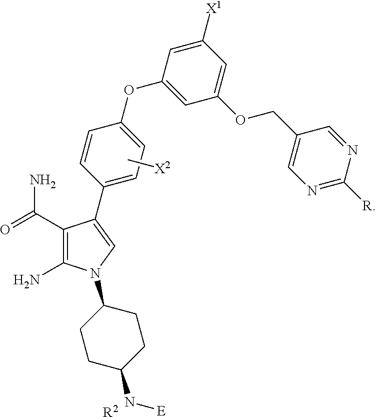 |

wherein:
X¹ and X² are independently selected from hydrogen and fluorine;
R² is selected from hydrogen and methyl;
E is selected from

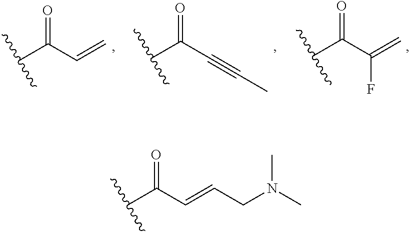

### and —CN, and

R is selected from hydrogen and methyl.

Compounds of the instant invention, where A is

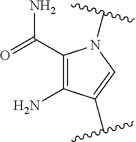 [SMILES: *c1cn(*)c(C(N)=O)c1N]

are prepared according to the following procedure:

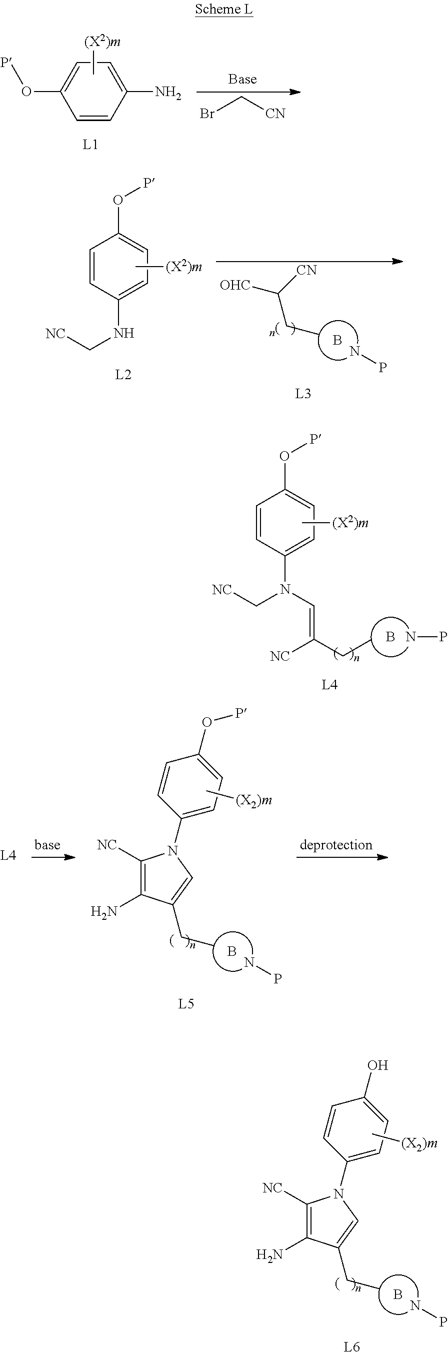

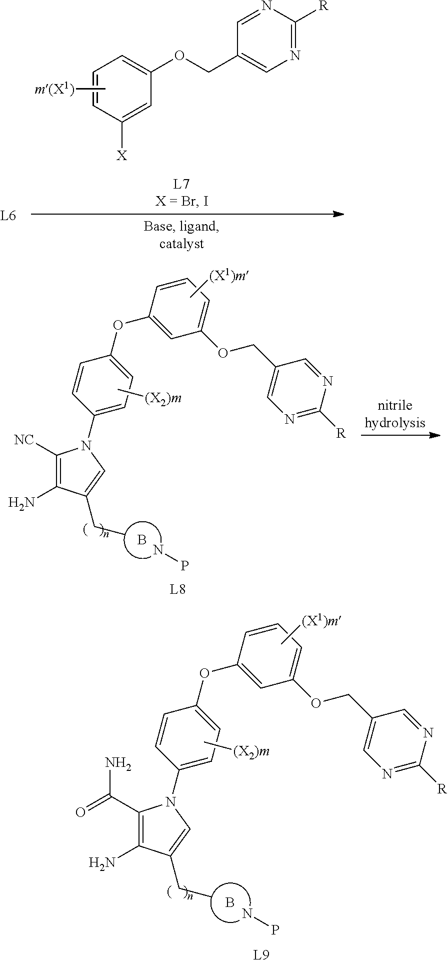

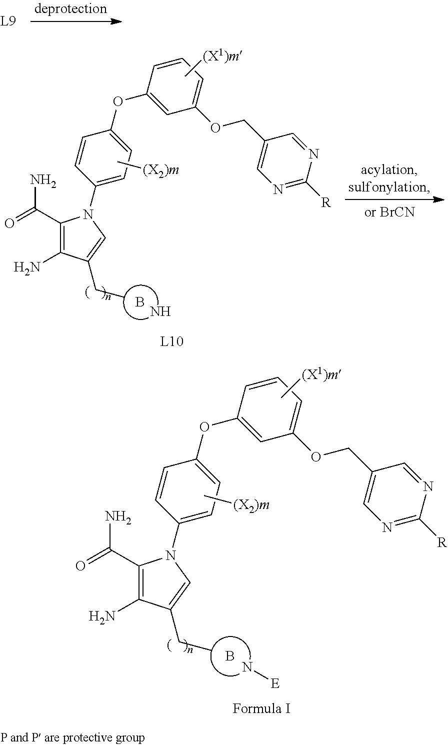

Compounds of the instant invention, where A is

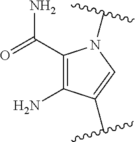 [SMILES: *c1cn(*)c(C(N)=O)c1N]

and

L-E is

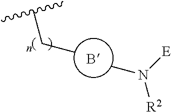

are prepared in a similar manner by substituting

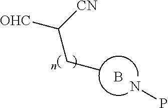

with

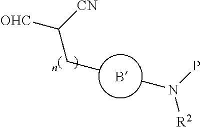

where rings B and B′, n, R, R² and E are as defined above.

Representative examples are listed below:

| Examples | |
|---|---|
| No. | Structure |
| 54 | 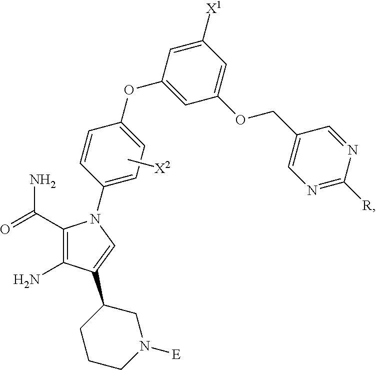 |
| 55 | 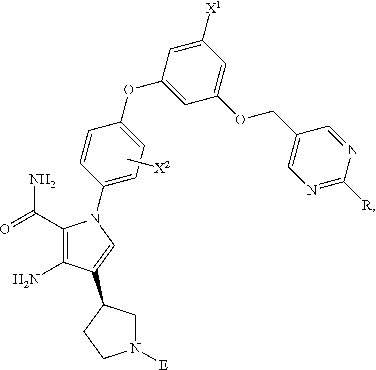 |
| 56 | 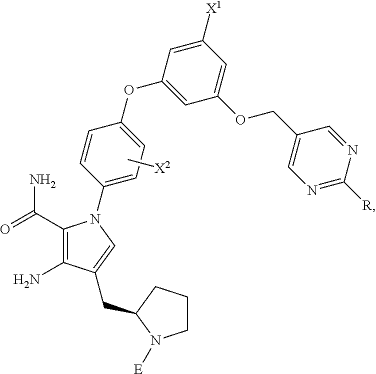 |
| 57 | 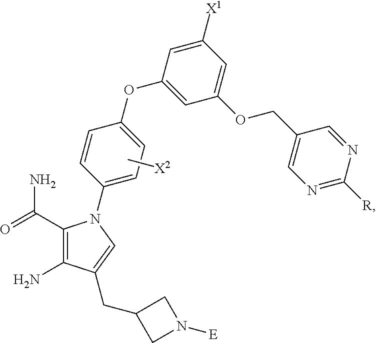 |
| 58 | 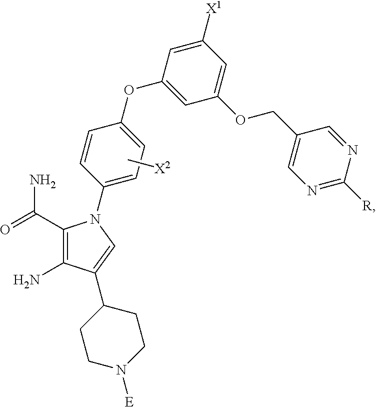 |
| 59 | 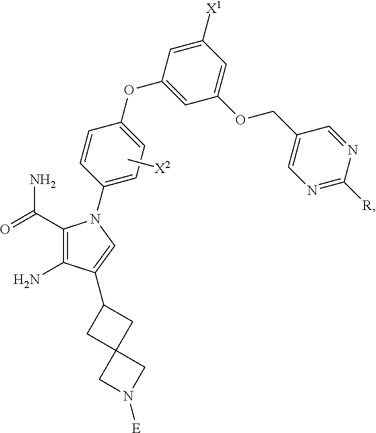 |
| 61 | 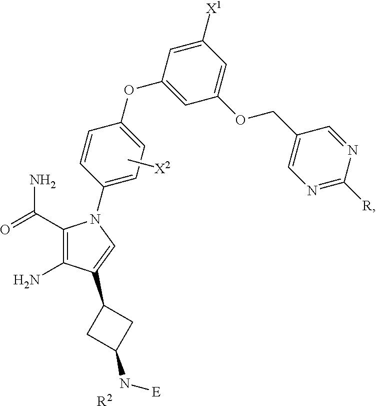 |
| 62 | 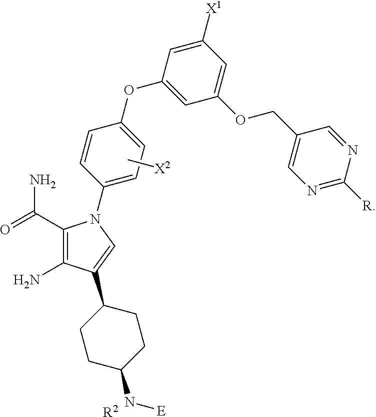 |

wherein:
X¹ and X²are independently selected from hydrogen and fluorine;
R² is selected from hydrogen and methyl;
E is selected from

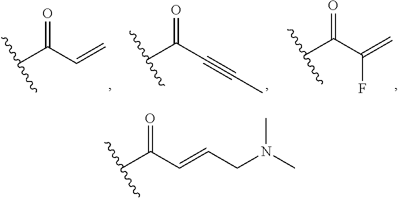

### and —CN, and

R is selected from hydrogen and methyl.

Compounds of the instant invention, where A is

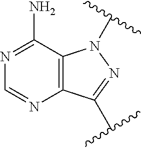 [SMILES: *c1nn(*)c2c(N)ncnc12]

are prepared according to the following procedure:

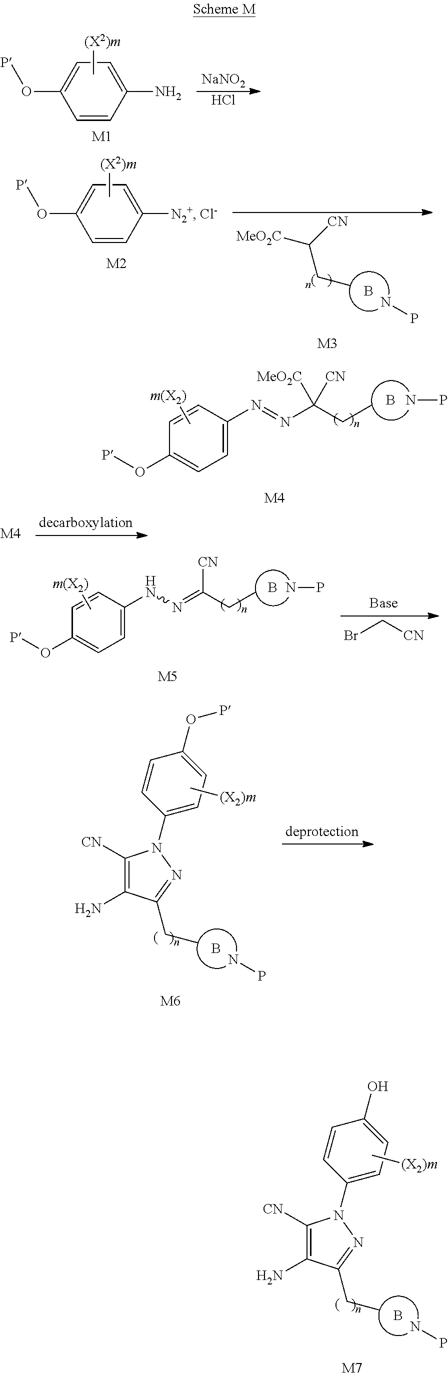

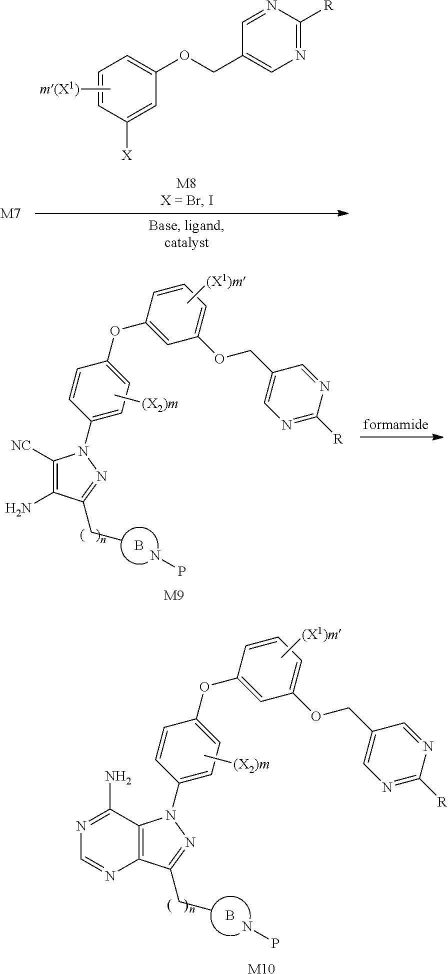

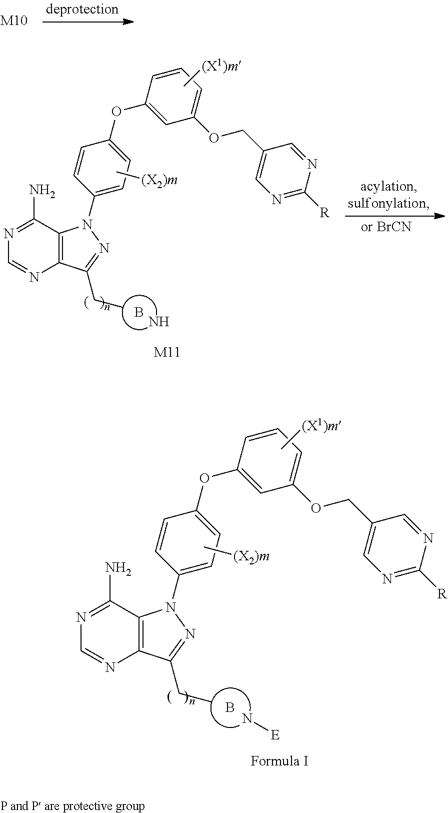

Compounds of the instant invention, where A is

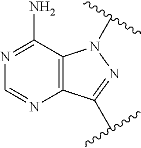 [SMILES: *c1nn(*)c2c(N)ncnc12]

and

L-E is

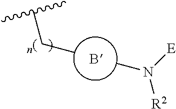

are prepared in a similar manner by substituting

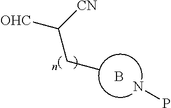

with

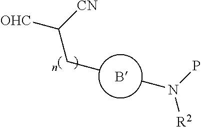

where rings B and B′, n, R, R² and E are as defined above.

Representative examples are listed below:

| Examples | |
|---|---|
| No. | Structure |
| 63 | 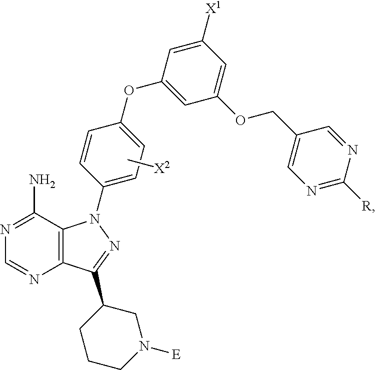 |
| 64 | 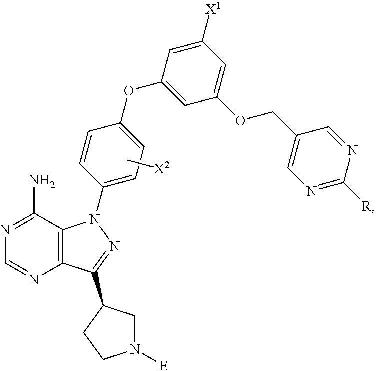 |
| 65 | 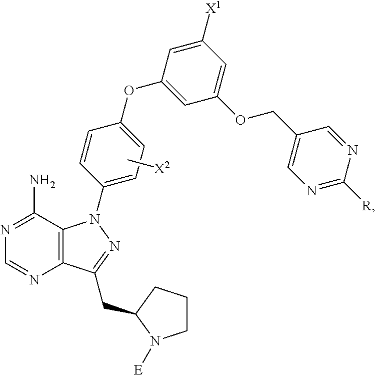 |
| 66 | 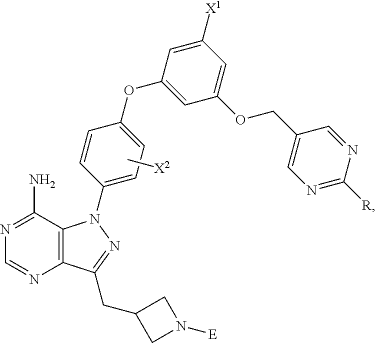 |
| 67 | 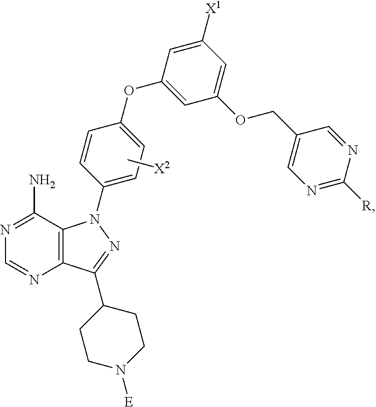 |
| 68 | 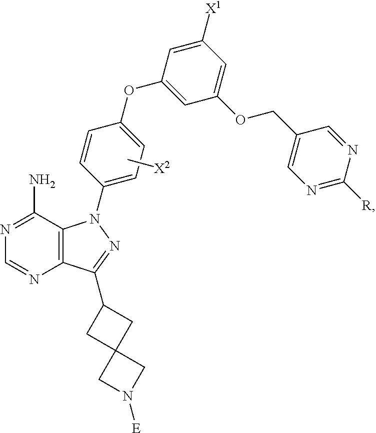 |
| 69 | 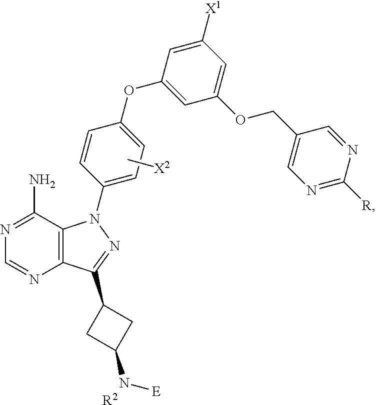 |
| 70 | 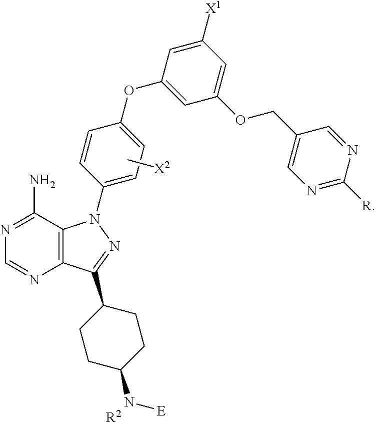 |

wherein:
X¹ and X² are independently selected from hydrogen and fluorine;
R² is selected from hydrogen and methyl;
E is selected from

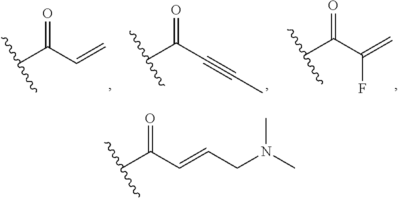

### and —CN, and

R is selected from hydrogen and methyl.

Compounds of the instant invention where A is

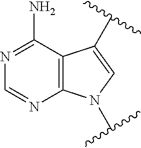 [SMILES: *c1cn(*)c2ncnc(N)c12]

can be prepared according to the following procedures:

Method A:

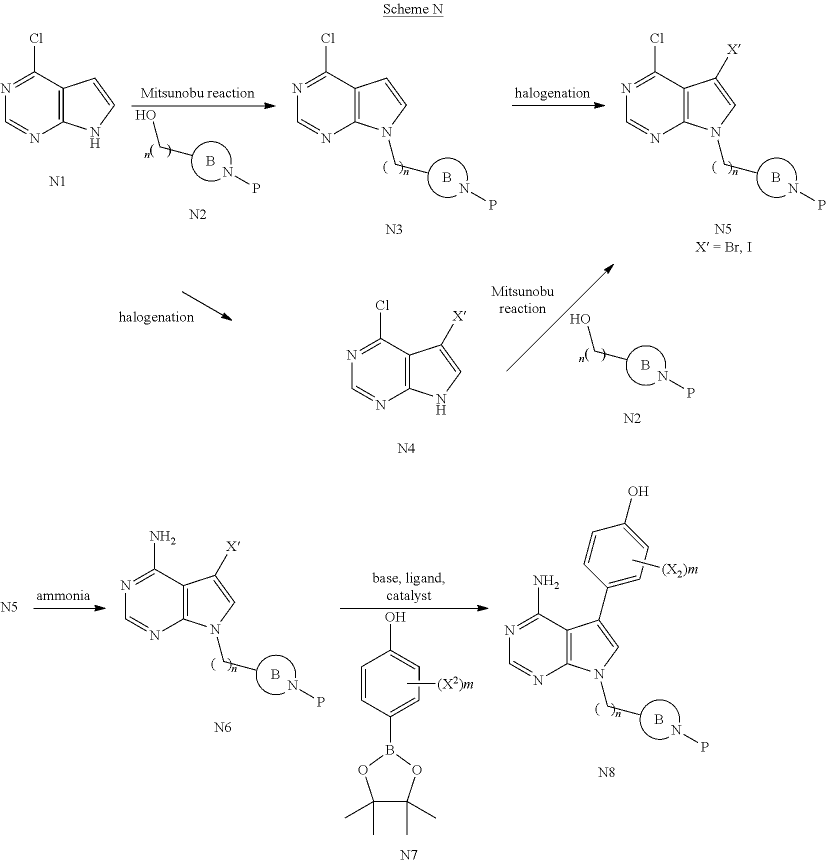

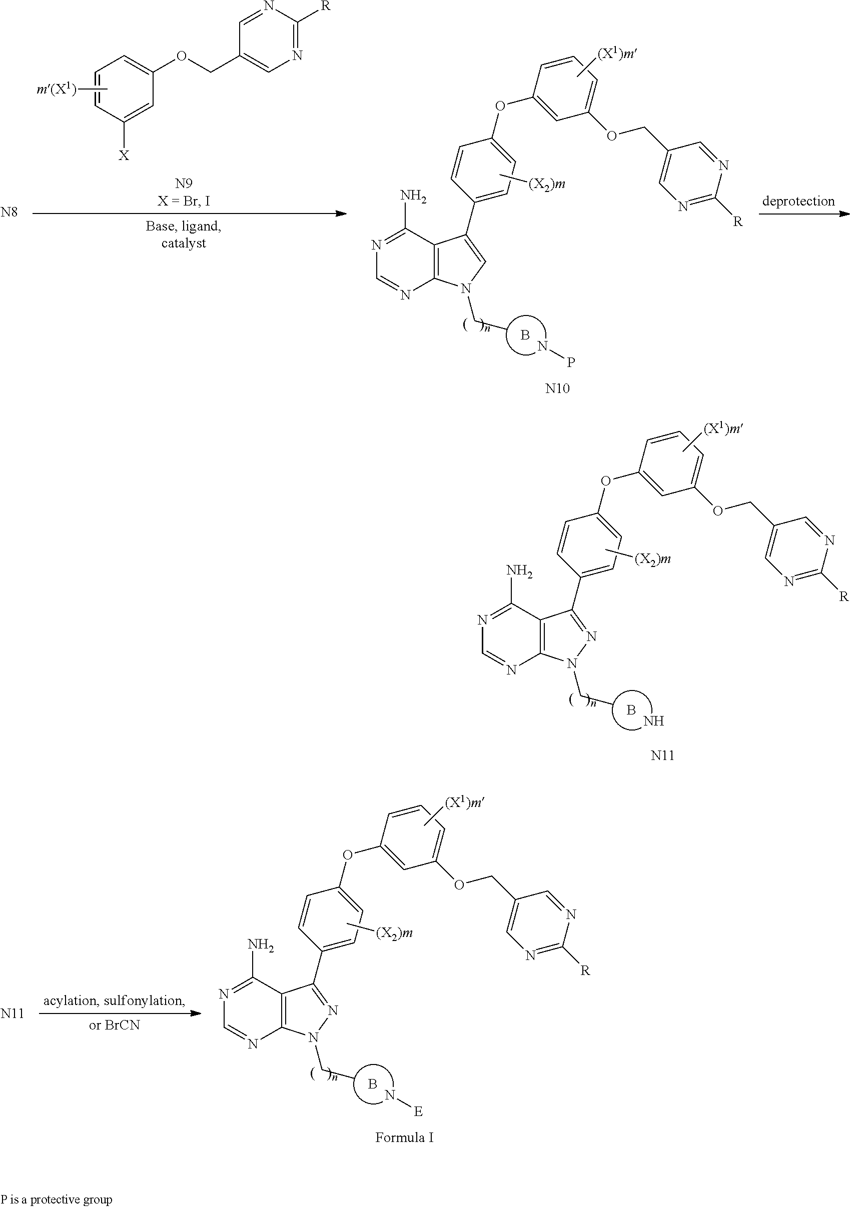

Method B:

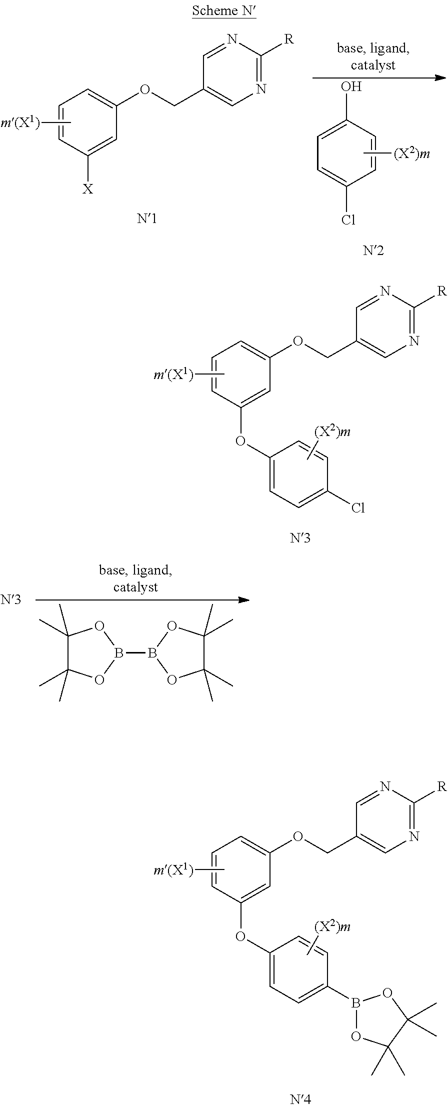

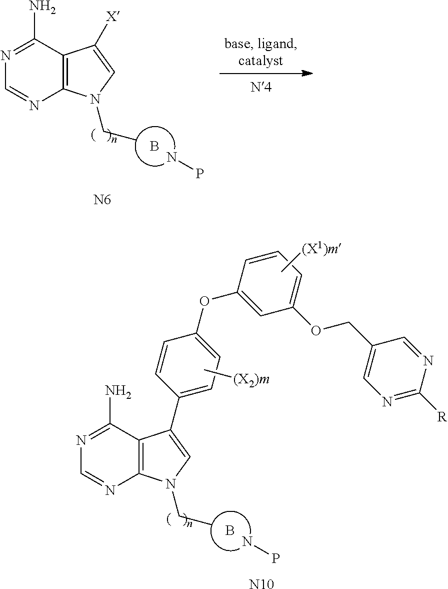

Compounds of the instant invention, where A is

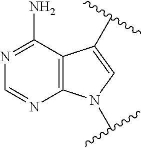 [SMILES: *c1cn(*)c2ncnc(N)c12]

and L-E is

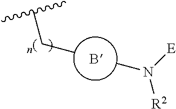

are prepared in a similar manner by substituting

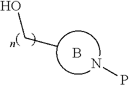

with

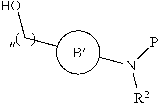

where rings B and B′, n, R, R² and E are as defined above.

Representative examples are listed below:

| Examples | |
|---|---|
| No. | Structure |
| 71 | 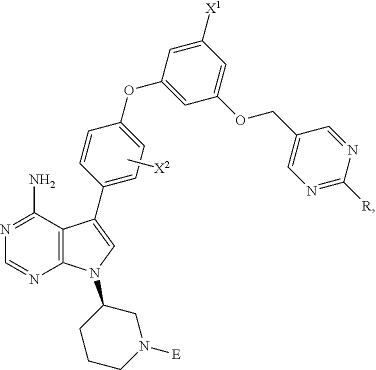 |
| 72 | 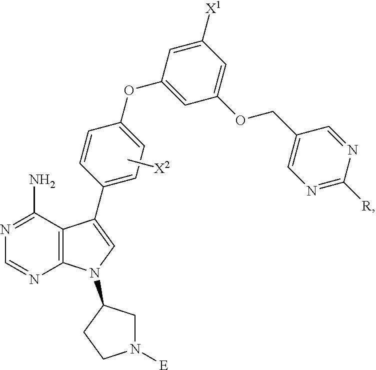 |
| 73 | 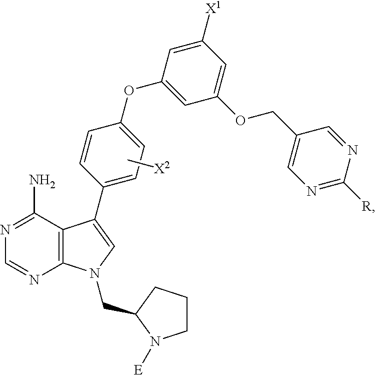 |
| 74 | 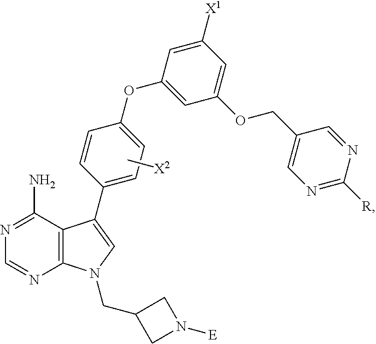 |
| 75 | 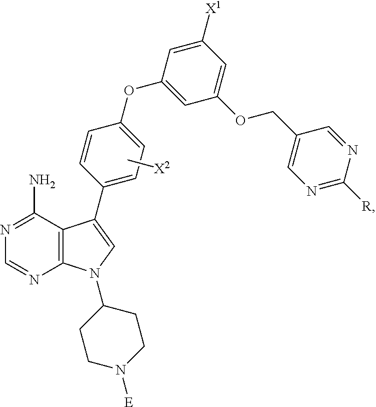 |
| 76 | 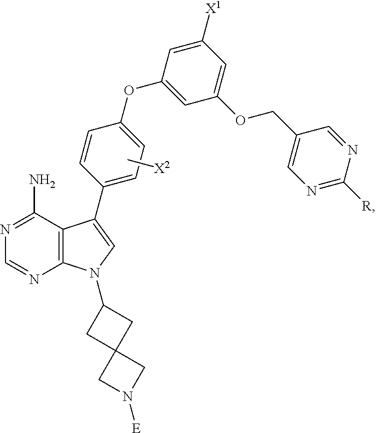 |
| 77 | 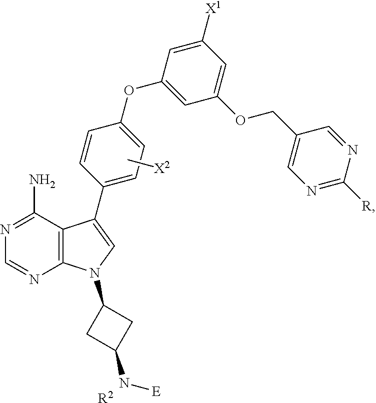 |
| 78 | 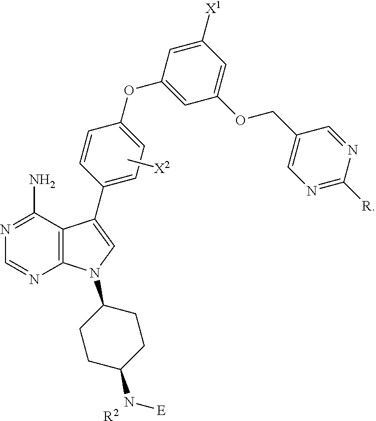 |

wherein:
X¹ and X² are independently selected from hydrogen and fluorine;
R² is selected from hydrogen and methyl;
E is selected from

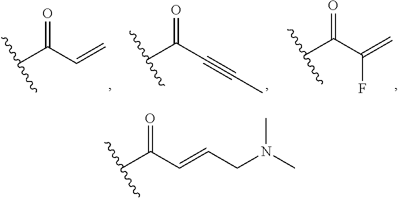

### and —CN, and

R is selected from hydrogen and methyl.

Compounds of the instant invention, where A is

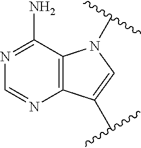 [SMILES: *c1cn(*)c2c(N)ncnc12]

are prepared according to the following procedures:

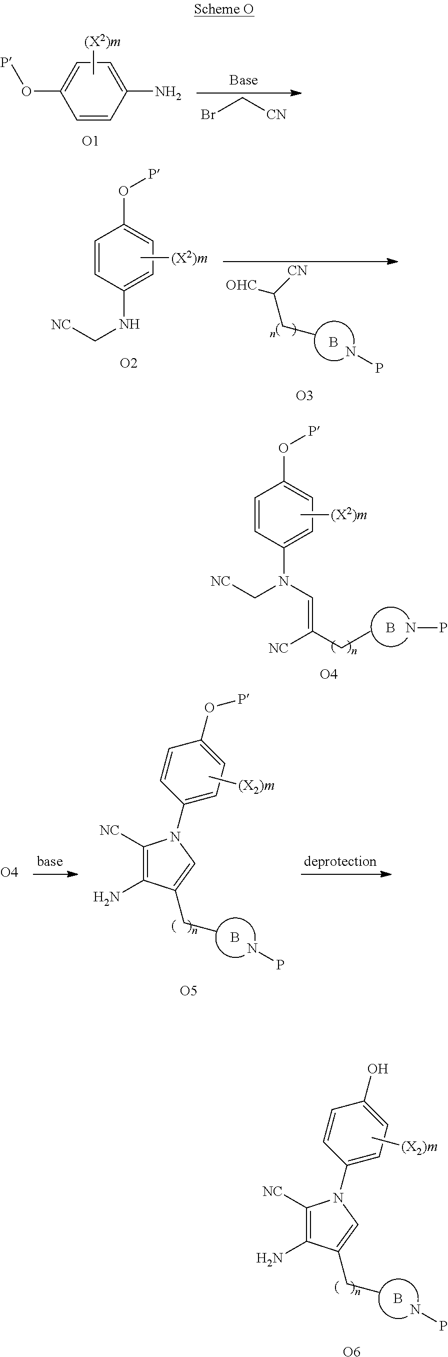

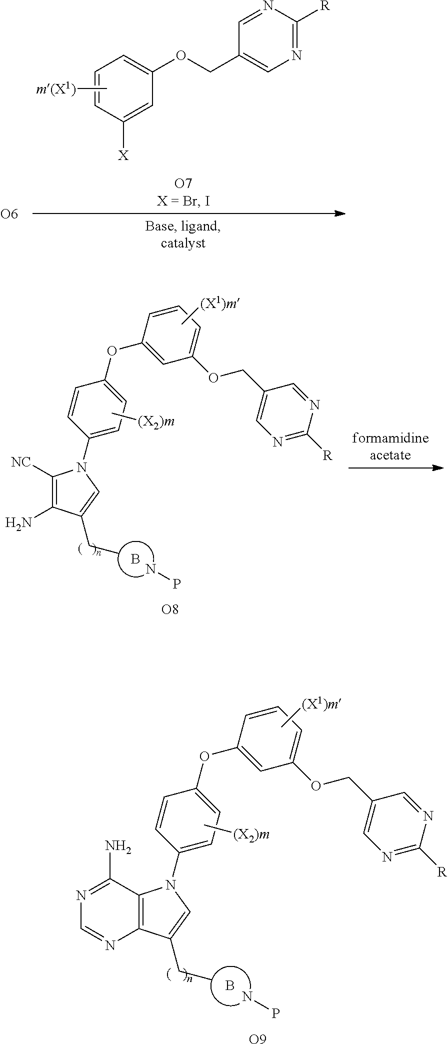

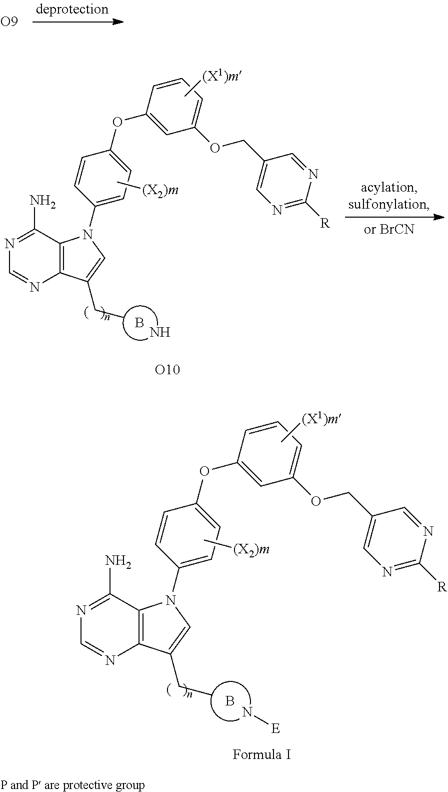

Compounds of the instant invention, where A is

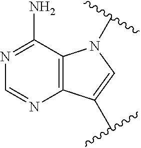 [SMILES: *c1cn(*)c2c(N)ncnc12]

and L-E is

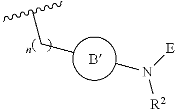

are prepared in a similar manner by substituting

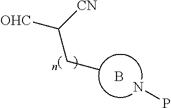

with

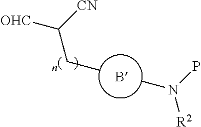

where rings B and B′, n, R, R² and E are as defined above.

Representative examples are listed below:

| Exam- | |
|---|---|
| ples | |
| No. | Structure |
| 79 | 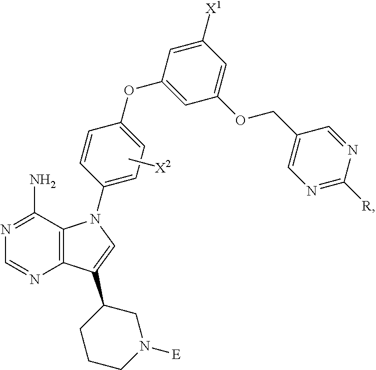 |
| 80 | 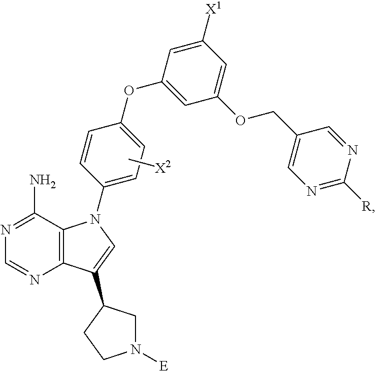 |
| 81 | 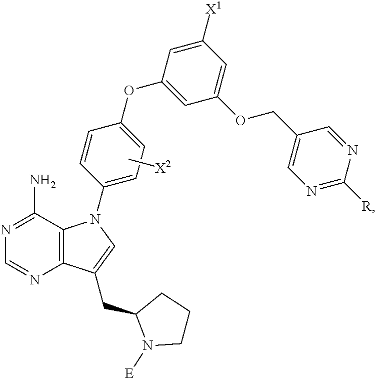 |
| 82 | 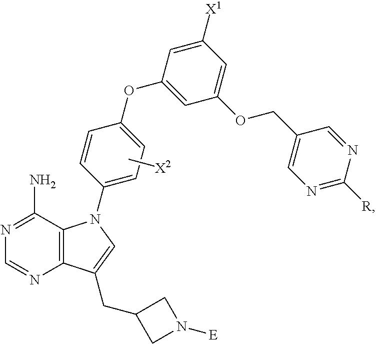 |
| 83 | 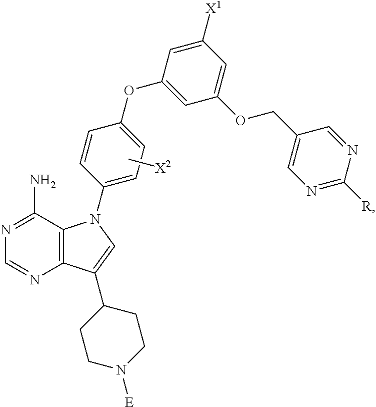 |
| 84 | 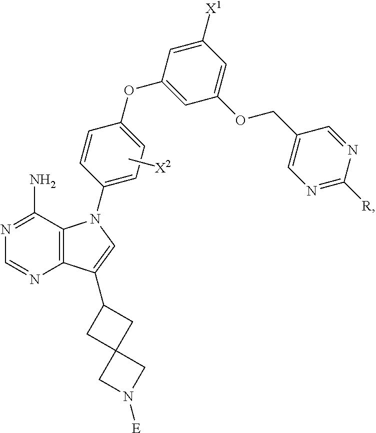 |
| 85 | 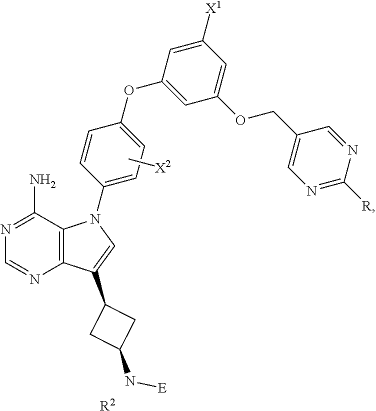 |
| 86 | 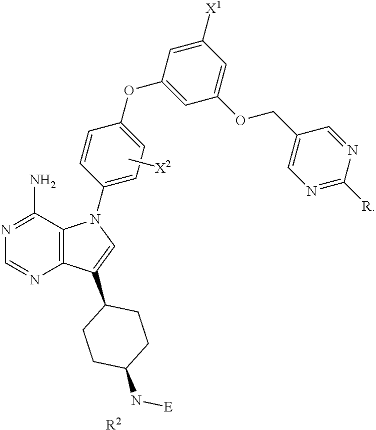 |

wherein:
X¹ and X²are independently selected from hydrogen and fluorine;
R² is selected from hydrogen and methyl;
E is selected from

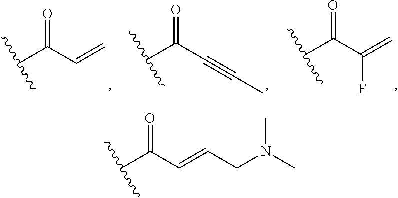

### and —CN, and

R is selected from hydrogen and methyl.

Compounds of the instant invention, where A is

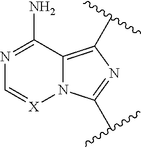

and X═CH are prepared according to the following procedures:

Method A:

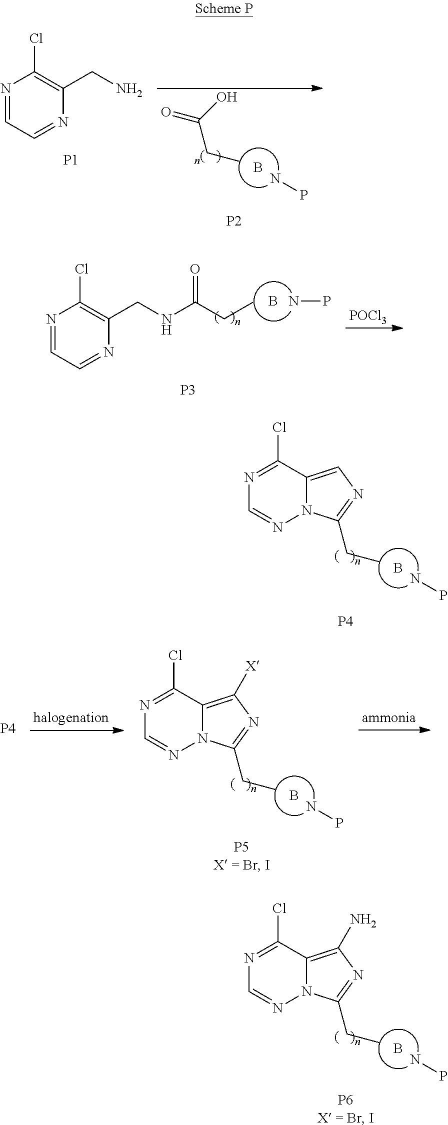

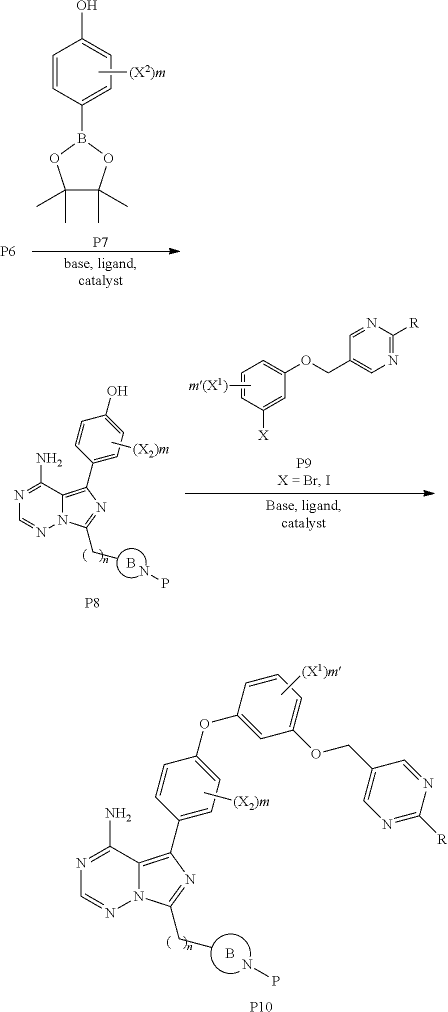

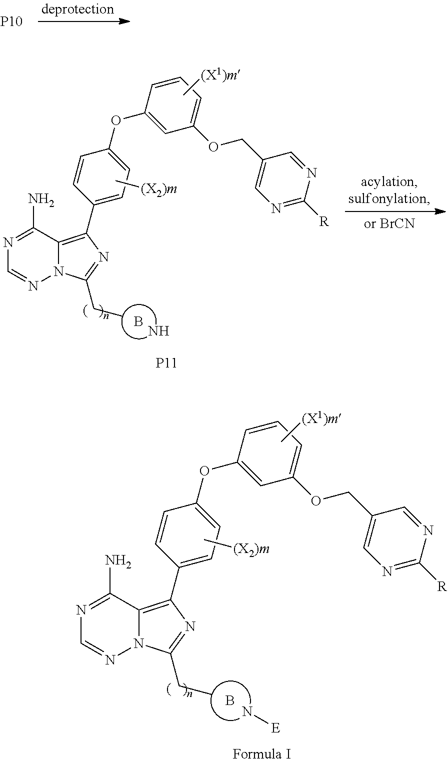

Method B:

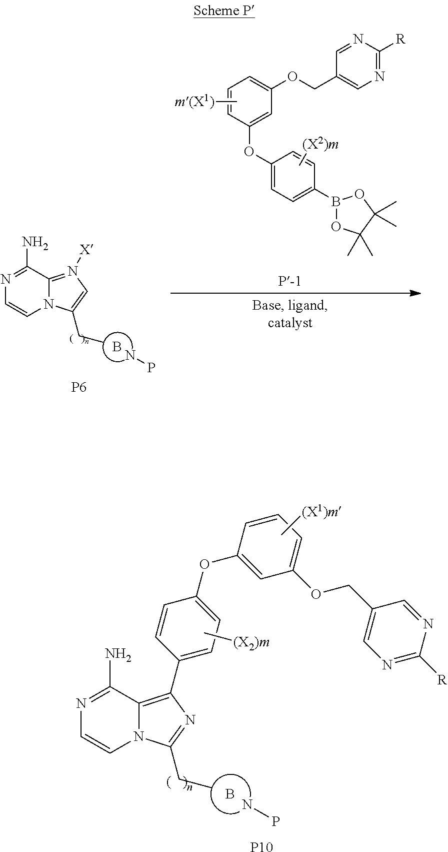

Compounds of the instant invention, where A is

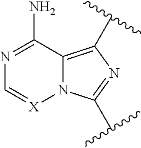

and L-E is

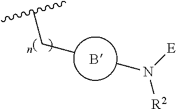

are prepared in a similar manner by substituting

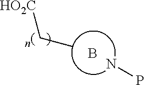

with

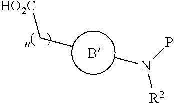

where rings B and B′, n, R, R² and E are as defined above.

Compounds of the instant invention, where A is

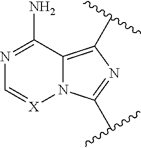

and X═N are prepared in a similar manner by substituting

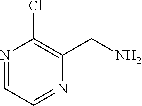

with

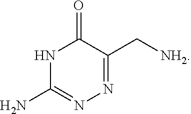

Representative examples are listed below:

| Ex- | |
|---|---|
| amples | |
| No. | Structure |
| 87 | 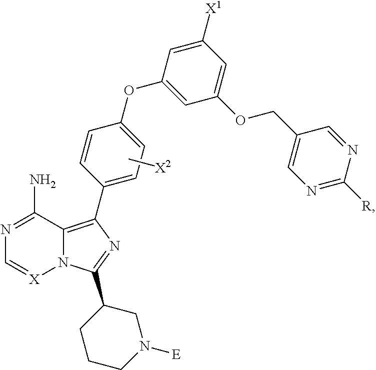 |
| 88 | 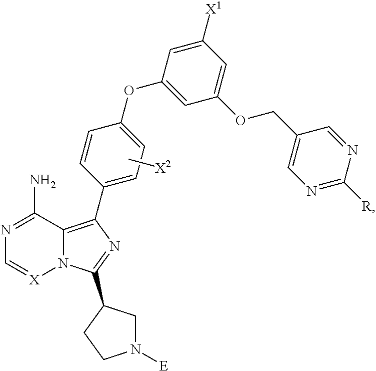 |
| 89 | 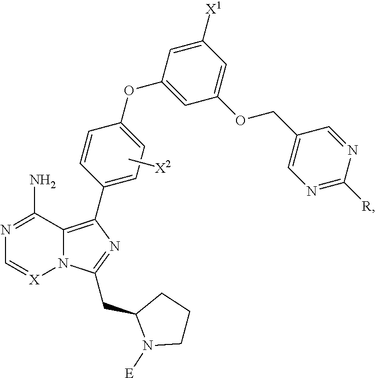 |
| 90 | 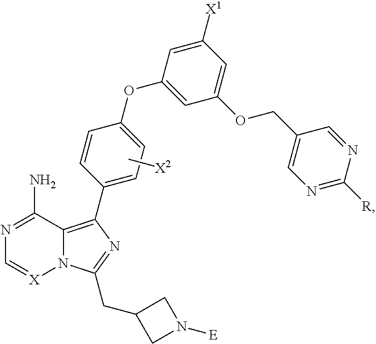 |
| 91 | 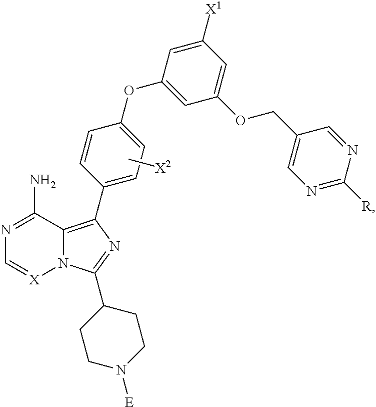 |
| 92 | 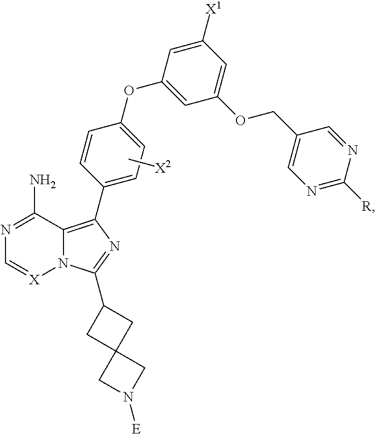 |
| 93 | 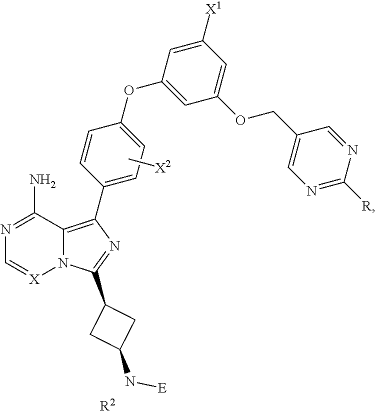 |
| 94 | 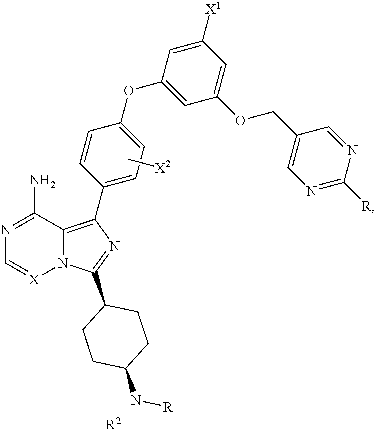 |

wherein:
X¹ and X² are independently selected from hydrogen and fluorine;
R² is selected from hydrogen and methyl;
X is selected from CH and N;
E is selected from

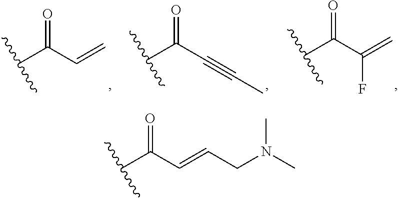

### and —CN, and

R is selected from hydrogen and methyl.

Compounds of the instant invention, where A is

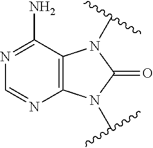 [SMILES: *n1c(=O)n(*)c2c(N)ncnc21]

are prepared according to the following procedures:

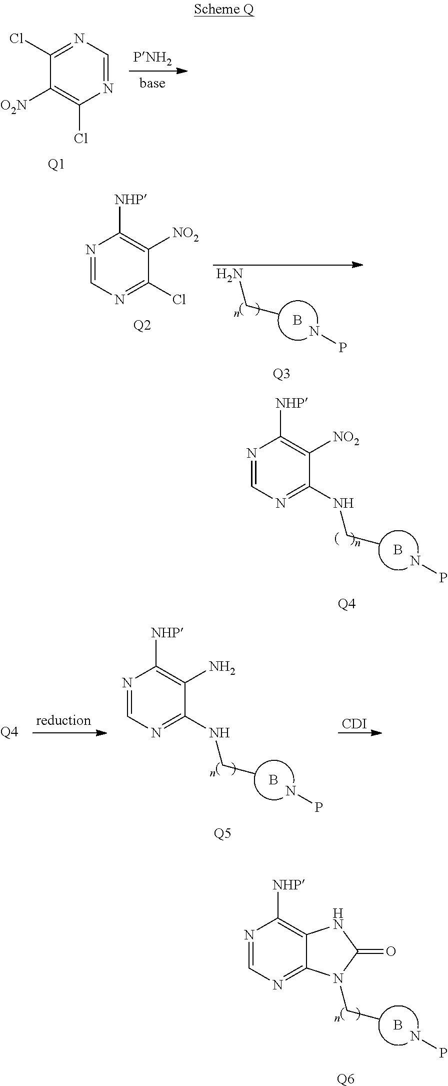

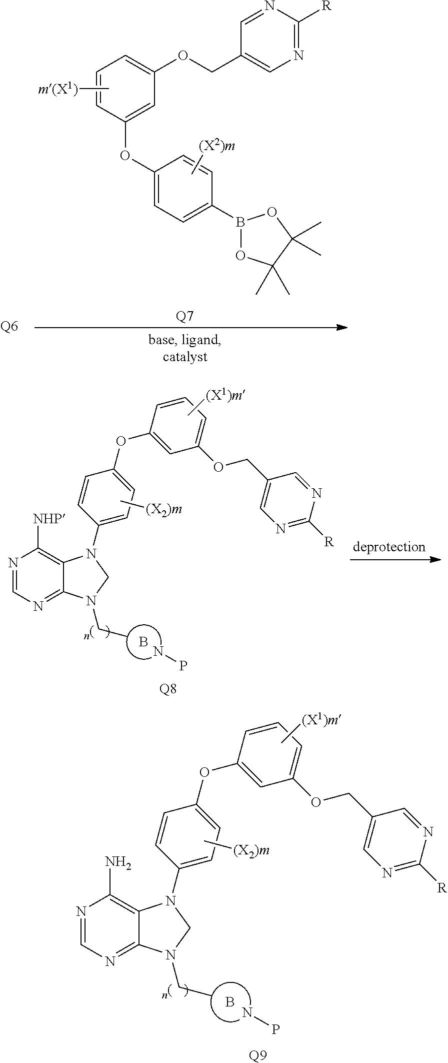

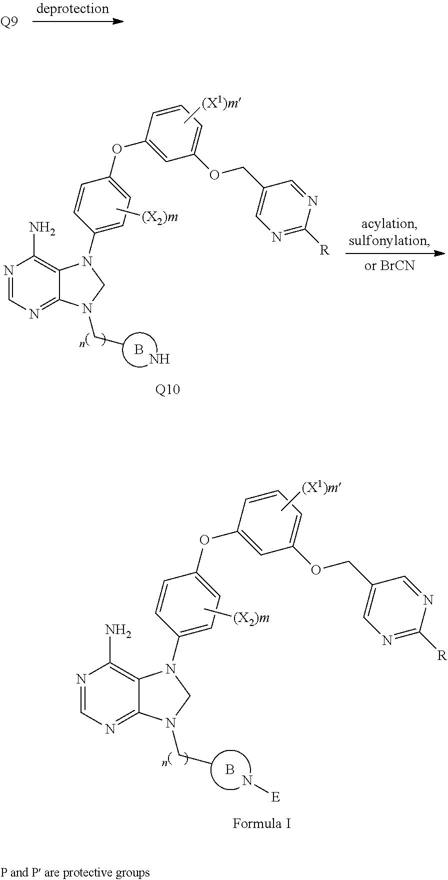

Compounds of the instant invention, where A is

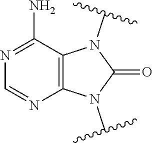 [SMILES: *n1c(=O)n(*)c2c(N)ncnc21]

and L-E is

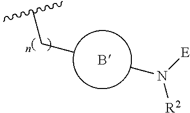

are prepared in a similar manner by substituting

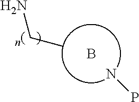

with

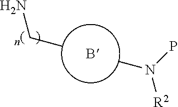

where rings B and B′, n, R, R² and E are as defined above.

Representative examples are listed below:

| Ex- | |
|---|---|
| amples | |
| No | Structure |
| 95 | 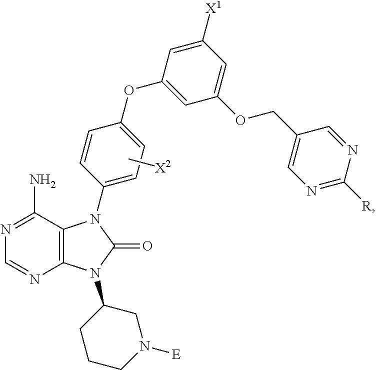 |
| 96 | 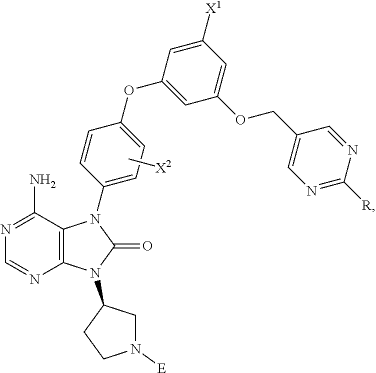 |
| 97 | 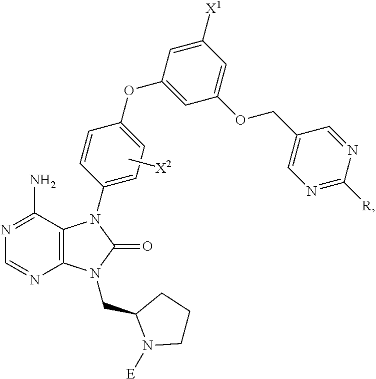 |
| 98 | 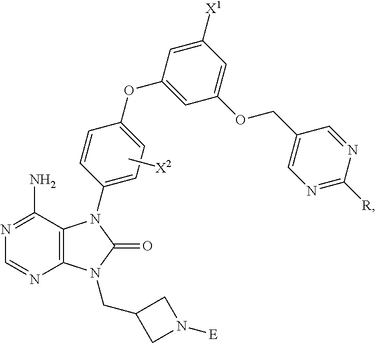 |
| 99 | 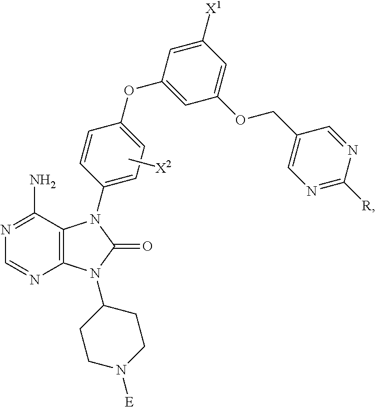 |
| 100 | 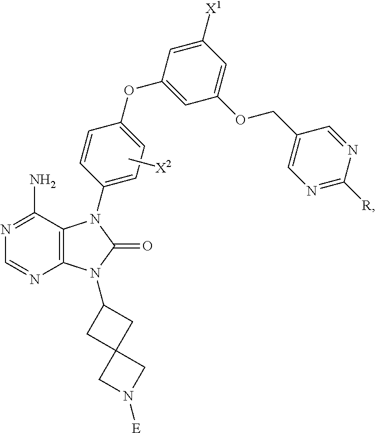 |
| 101 | 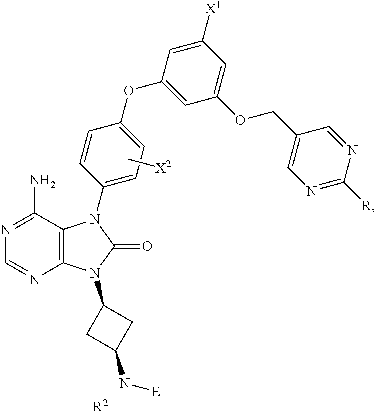 |
| 102 | 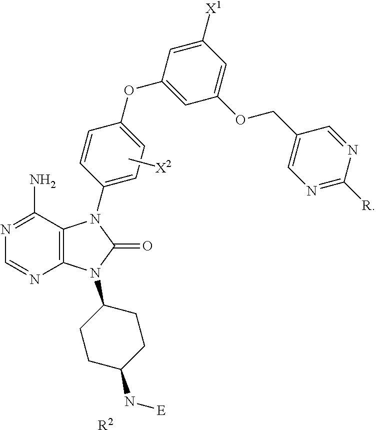 |

wherein:
X¹ and X² are independently selected from hydrogen and fluorine;
R² is selected from hydrogen and methyl;
E is selected from

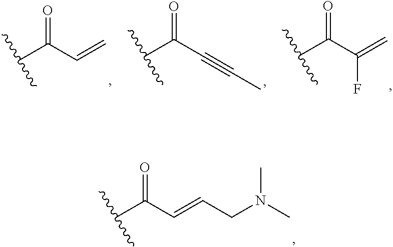

### and —CN, and

R is selected from hydrogen and methyl.

Biological Assays

Assays for determining the in vitro activity of kinase inhibitors are described in more detail in the accompanying examples.

Kinase Inhibition

BTK Kinase Inhibition Assays

In vitro potency of selected compound was defined against human BTK kinase (hBTK) using Kinase Profiler radiometric protein kinase assays performed at Eurofins Pharma Discovery Services UK Limited.

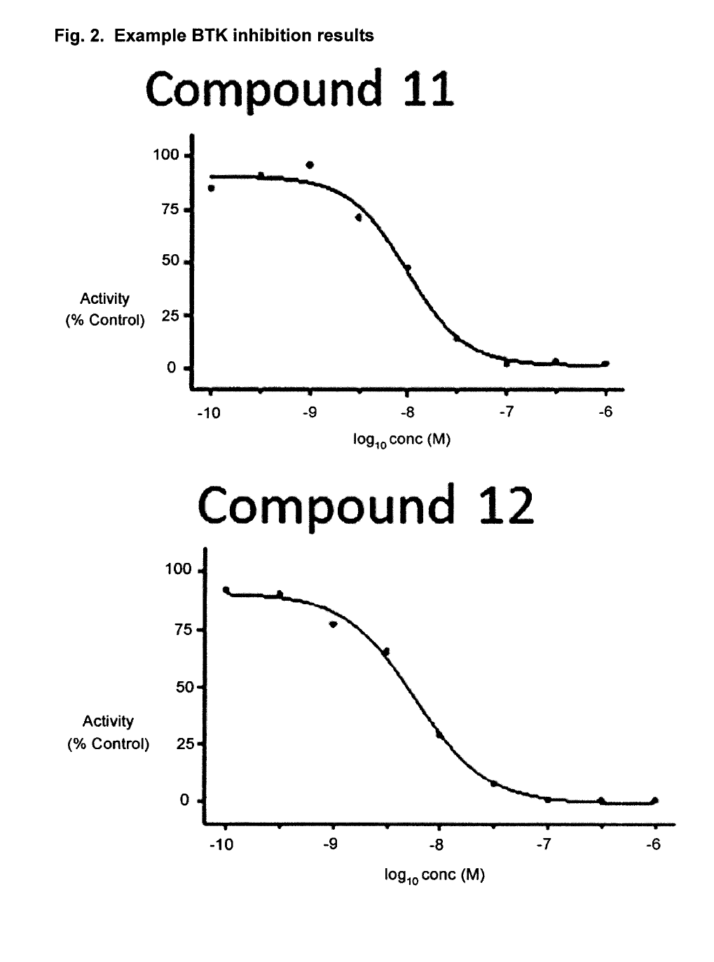

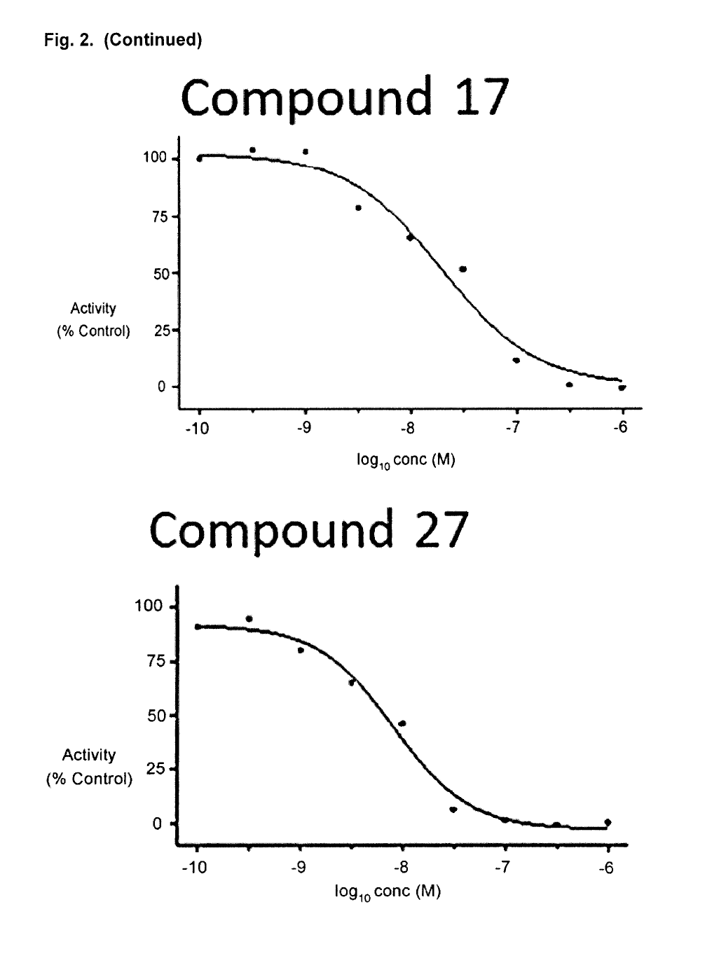

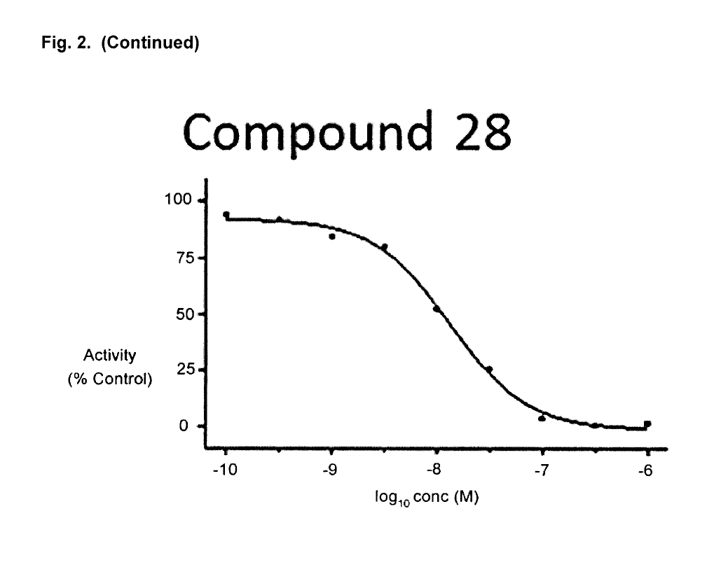

hBTK kinase is diluted in buffer and all compounds were prepared to 50× final assay concentration in 100% DMSO. This working stock of the compound was added to the assay well as the first component in the reaction, followed by the remaining components as detailed in the assay protocol listed above. The reaction was initiated by the addition of the MgATP mix. The kinase reaction was performed at room temperature for 40 minutes in presence of 250 μM substrate, 10 mM MgAcetate, [γ-33P-ATP] (specific activity approx. 500 cpm/pmol, concentration as required) and variable test article concentrations. The ATP concentrations in the assays were with 15 μM of the apparent. The reaction was stopped by the addition of 3% phosphoric acid solution. 10 μL of the reaction is then spotted onto a P30 filtermat and washed three times for 5 minutes in 75 mM phosphoric acid and once in methanol prior to drying and scintillation counting. In addition positive control wells contain all components of the reaction, except the compound of interest; however, DMSO (at a final concentration of 2%) were included in these wells to control for solvent effects as well as blank wells contain all components of the reaction, with a reference inhibitor replacing the compound of interest. This abolishes kinase activity and establishes the base-line (0% kinase activity remaining). Example results are presented in FIG. 2. The potency of each compound was reported by estimating the IC₅₀and shown in Table 2.

TABLE 2
Results of BTK inhibition

| Compound | IC50 (nM) |
|---|---|
| 1 | 76 |
| 2 | 119 |
| 3 | 129 |
| 4 | 125 |
| 5 | 126 |
| 6 | 55 |
| 7 | 16 |
| 8 | 11 |
| 9 | 40 |
| 10 | 8 |
| 11 | 10 |
| 12 | 6 |
| 13 | 35 |
| 14 | 25 |
| 15 | 53 |
| 16 | 7 |
| 17 | 19 |
| 18 | 14 |
| 19 | 7 |
| 20 | 6 |
| 21 | 13 |
| 22 | 6 |
| 23 | 9 |
| 24 | 26 |
| 25 | 17 |
| 26 | 24 |
| 27 | 8 |
| 28 | 13 |
| 29 | 8 |
| 30 | 9 |
| 31 | 10 |
| 32 | 8 |

BTK Enzyme Off-Rate Determination

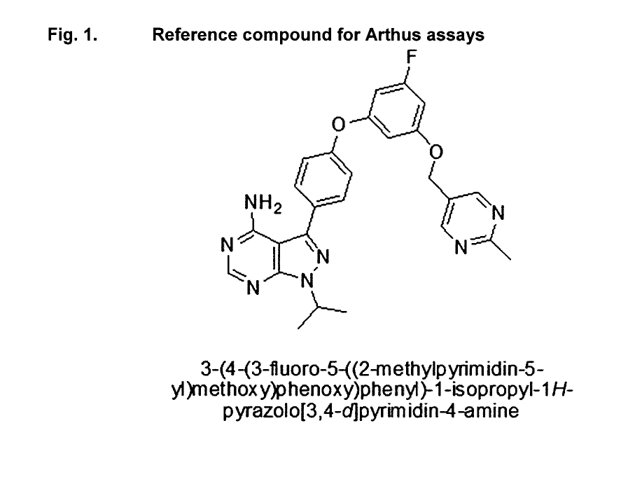

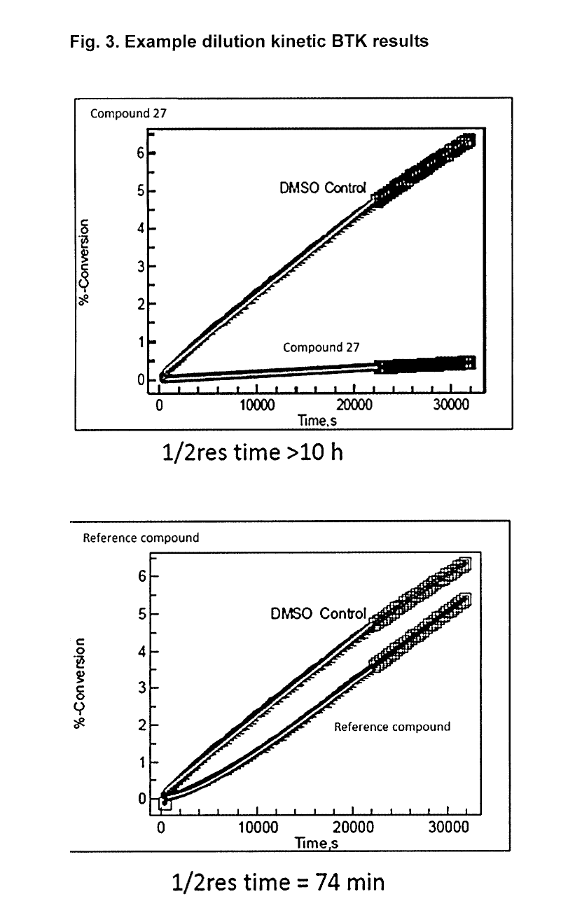

10-30 nM compound was pre-incubated with 7 nM enzyme in the presence of 1000 uM ATP for 1 h. The [I]-[E] complex was diluted 250× into an assay buffer with 1000 uM ATP and 2uM substrate peptide. Enzyme activity in the diluted samples was monitored in real time assay over period of 8 h. The progress curves were fit with time dependent equation to determine apparent Kobs and residence time of the compound. The reference compound (FIG. 1) has a residence half life of 74 mintues on BTK. Consistent with covalent inhibition of BTK. Data show that compound 27 has a residence half life of greater than 10 hours (FIG. 3). These data are consistent with covalent inhibition of BTK by compound 27.

TABLE 3
Results of dilution kinetic experiments

| Compound | Residence time half life (1/Kobs) | |
|---|---|---|
| Reference compound | 74 | minutes |
| Compound 27 | >10 | hours |

Cellular Assays Splenic Cell Proliferation Assay

Proliferation of splenocytes in response to anti-IgM can be blocked by inhibition of Btk.

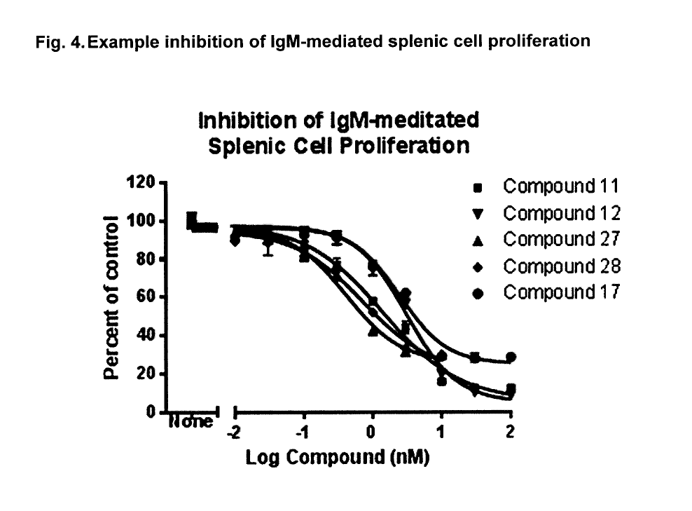

Splenocytes were obtained from 6 week old male CD1 mice (Charles River Laboratories Inc.). Mouse spleens were manually disrupted in PBS and filtered using a 70 um cell strainer followed by ammonium chloride red blood cell lysis. Cells were washed, resuspended in Splenocyte Medium (HyClone RPMI supplemented with 10% heat-inactivated FBS, 0.5× non-essential amino acids, 10 mM HEPES, 50 uM beta mercaptoethanol) and incubated at 37° C., 5% CO₂ for 2 h to remove adherent cells. Suspension cells were seeded in 96 well plates at 50,000 cells per well and incubated at 37° C., 5% CO₂ for 1 h. Splenocytes were pre-treated in triplicate with 10,000 nM curves of Formula 1 compounds for 1 h, followed by stimulation of cell proliferation with 2.5 ug/ml anti-IgM F(ab′)₂ (Jackson ImmunoResearch) for 72 h. Cell proliferation was measured by Cell Titer-Glo Luminescent Assay (Promega). Example results are presented in FIG. 4. EC₅₀ values (50% proliferation in the presence of compound as compared to vehicle treated controls) were calculated from dose response compound curves using GraphPad Prism Software EC₅₀ values are reported in Table 3:

TABLE 3
Results of inhibition of splenic cell proliferation

| Compound | EC₅₀ (nM) |
|---|---|
| 1 | 12.2 |
| 2 | a |
| 3 | 12.8 |
| 4 | a |
| 5 | 7.8 |
| 6 | 11.5 |
| 7 | 48.7 |
| 8 | 4.6 |
| 9 | 6.4 |
| 10 | 9.8 |
| 11 | 1.5 |
| 12 | 3.1 |
| 13 | 48.7 |
| 14 | 2.5 |
| 15 | 8.7 |
| 16 | 4.1 |
| 17 | 6.9 |
| 18 | 5.8 |
| 19 | 13.4 |
| 20 | 2.0 |
| 21 | 32.9 |
| 22 | 2.7 |
| 23 | 4.9 |
| 24 | 10.8 |
| 25 | 4.3 |
| 26 | 4.6 |
| 27 | 0.8 |
| 28 | 1.1 |
| 29 | 1.0 |
| 30 | 1.0 |
| 31 | 1.4 |
| 32 | 1.5 |
| 33 | 47.3 |
| 34 | 1.5 |
| 35 | 1.0 |

a = EC50 > 100 nM and not determined

TMD-8 Survival Assay

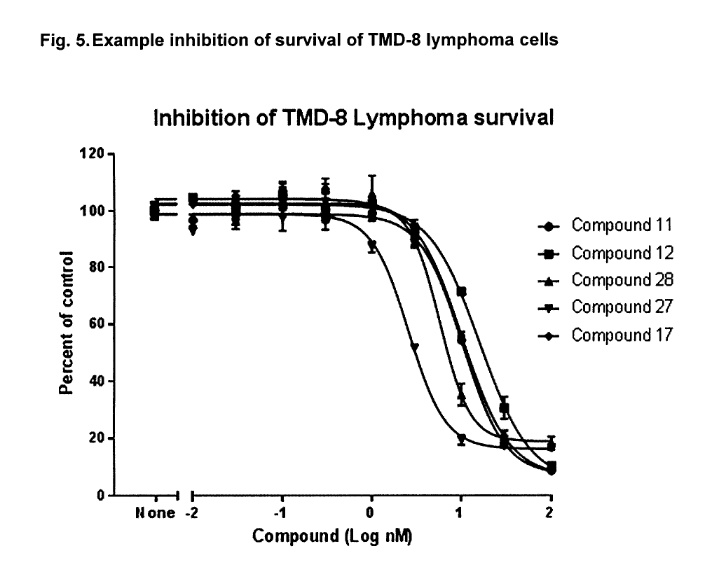

BTK inhibitors affect the survival of B-cell lymphomas. TMD-8 human activated B cell diffuse large B cell lymphoma cells were seeded in 96-well plates at a density of 20,000 cells/well in HyClone RPMI supplemented with 10% FBS (Fisher)/1% Penicillin/Streptomycin (HyClone) and incubated at 37° C., 5% CO₂. Cells were treated in triplicate with 1,000 nM or 100 nM curves of compounds for 72 h. Cell survival was measured by Cell Titer-Glo Luminescent Assay (Promega). Example results are presented in FIG. 5. EC₅₀ values (50% proliferation in the presence of compound as compared to vehicle treated controls) were calculated from dose response compound curves using GraphPad Prism Software.

TABLE 4
Results of TMD-8 survival assay

| Compound | EC₅₀ (nM) |
|---|---|
| 1 | 34.7 |
| 2 | a |
| 3 | 45.3 |
| 4 | a |
| 5 | 48.8 |
| 6 | 36.1 |
| 7 | a |
| 8 | 13.5 |
| 9 | 18.7 |
| 10 | 75.5 |
| 11 | 13.1 |
| 12 | 16.5 |
| 13 | 79.3 |
| 14 | 13.8 |
| 15 | 56.1 |
| 16 | 12.1 |
| 17 | 14.5 |
| 18 | 14.4 |
| 19 | 53.3 |
| 20 | 8.6 |
| 21 | 64.5 |
| 22 | 13.4 |
| 23 | 13.9 |
| 24 | 20.6 |
| 25 | 19.8 |
| 26 | 18.1 |
| 27 | 4.7 |
| 28 | 8.4 |
| 29 | 6.6 |
| 30 | 5.1 |
| 31 | 8.9 |
| 32 | 8.5 |
| 33 | a |

a = EC50 > 100 nM and not determined

In Vivo Assays

Arthus Reverse Passive Anaphylaxis

BTK is an important component of the signalling pathways activated downstream of Fc receptor activation of immune complex acute vasculitis in the Arthus reverse passive anaphylaxis assay.

Female Balb/c mice (6-7 weeks on arrival) were habituated to the animal facility for at least 4 days. On the day of the experiment, animals were pre-treated (t=minus 1 h) with test article (30 mg/kg) or vehicle alone by gavage (PO). At t=0, animals were injected intravenously (IV; 0.1 mL/mouse) with saline containing chicken ovalbumin and Evan's blue (10 mg/mL of each). Ten minutes later (t=10 min), animals were anesthesized with isoflurane, the dorsal surface was shaved and rabbit anti-chicken ovalbumin antibody was then injected intradermally at one site on the right side of the animal (25 μg in 30 μL). The same amount of isotype control antibody was then injected on the left side.

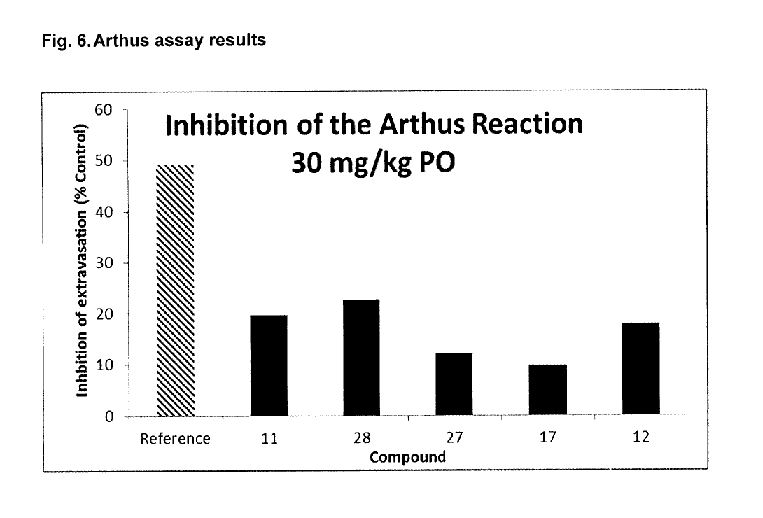

The animals were then returned to their home cage and skin punches (8 mm) were collected from each injection site four hours later. The samples were placed in 1 mL formamide overnight at 80 degrees C. (1 skin biopsy per 1 mL formamide in a glass tube). The amount of Evan's blue in the formamide solution was then assessed by spectrophotometry (630 nm) as a measure of serum extravasation into the dermis. Compound activity was compared with a reference compound FIG. 1. Inhibition of the arthus reaction relative to vehicle treated control is presented in FIG. 6.

1. A compound of Formula I: 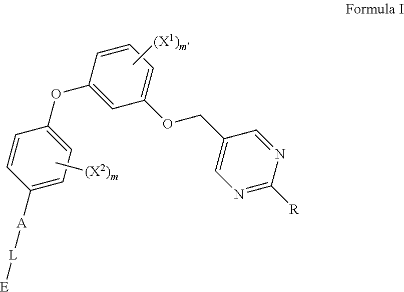
or a pharmaceutically acceptable salt, tautomer, prodrug, complex or biologically active metabolite thereof,
wherein
X¹ and X² are independently selected from hydrogen and halogen;
m is an integer from 0 to 4;
m′ is an integer from 0 to 4;
R is hydrogen or methyl;
A is either: 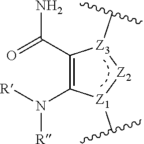
wherein the dashed line is independently an optional bond;
R′ and R″ are independently selected from hydrogen, substituted or unsubstituted alkyl, substituted or unsubstituted heteroalkyl, substituted or unsubstituted cycloalkyl, substituted or unsubstituted heterocyclyl, substituted or unsubstituted aryl, substituted or unsubstituted heteroaryl, substituted or unsubstituted aralkyl, or substituted or unsubstituted heteroaralkyl;
Z₁ and Z₃ are independently selected from C or N; and
Z₂ is selected from N or CR¹;
provided that at least one and no more than two of Z₁, Z₂ and Z₃ are simultaneously N;
or 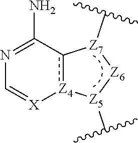
wherein the dashed lines are independently an optional bond;
Z₄, Z₅, and Z₇ are independently selected from C or N;
Z₆ is selected from N, C(O) or CR¹;
X is selected from N or CH;
provided that at least one and no more than two of Z₄, Z₅, Z₆ and Z₇ are simultaneously N; and
R¹ is selected from the group consisting of hydrogen, halogen, substituted or unsubstituted alkyl, substituted or unsubstituted heteroalkyl, substituted or unsubstituted cycloalkyl, substituted or unsubstituted heterocyclyl, substituted or unsubstituted aryl, substituted or unsubstituted heteroaryl, substituted or unsubstituted aralkyl, and substituted or unsubstituted heteroaralkyl;
L is selected from the group consisting of substituted or unsubstituted alkyl, substituted or unsubstituted heteroalkyl, substituted or unsubstituted cycloalkyl, substituted or unsubstituted heterocyclyl, substituted or unsubstituted aryl, substituted or unsubstituted heteroaryl, substituted or unsubstituted aralkyl, substituted or unsubstituted heteroaralkyl, 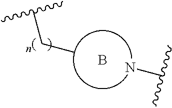
wherein
B is substituted or unsubstituted 3- to 8-membered nitrogen containing heterocyclic ring; and
n is an integer from 0 to 1;
or 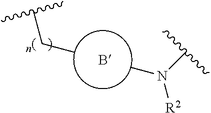
wherein
B′ is substituted or unsubstituted 3- to 8-membered cycloalkyl ring;
n is an integer from 0 to 1; and
R² is selected from hydrogen and lower alkyl;
E is selected from the group consisting of: 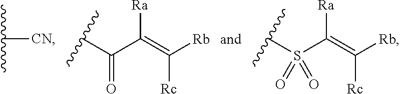
wherein
Ra, Rb and Rc are independently selected from hydrogen, halogen, —CN, substituted or unsubstituted alkyl, substituted or unsubstituted heteroalkyl, substituted or unsubstituted cycloalkyl, or substituted or unsubstituted heterocyclyl; or
Ra and Rb taken together with the carbon atoms to which they are attached form a 3- to 8-membered substituted or unsubstituted cycloalkyl ring, or form a 3- to 8-membered substituted or unsubstituted heterocyclic ring, and Rc is selected as above; or
Rb and Rc taken together with the carbon atom to which they are attached form a 3- to 8-membered substituted or unsubstituted cycloalkyl ring, or form a 3- to 8-membered heterocyclic ring, and Ra is selected as above; or
Ra and Rb taken together with the carbon atoms to which they are attached form a triple bond and Rc is selected as above.
provided A-L-E is

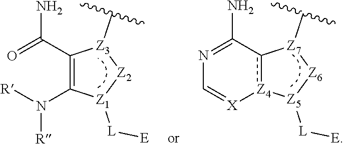

2. The compound according to claim 1, wherein A is selected from a group consisting of: 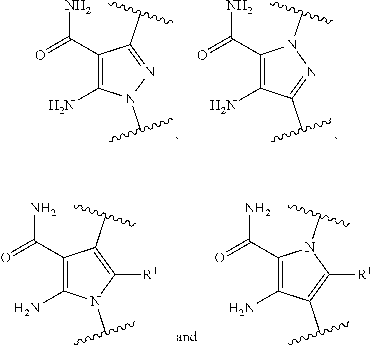
wherein R¹ is selected from hydrogen, halogen, substituted or unsubstituted alkyl, substituted or unsubstituted heteroalkyl, substituted or unsubstituted cycloalkyl, substituted or unsubstituted heterocyclyl, substituted or unsubstituted aryl, substituted or unsubstituted heteroaryl, substituted or unsubstituted aralkyl, or substituted or unsubstituted heteroaralkyl.

3. The compound according to claim 2, wherein R¹ is hydrogen.

4. The compound according to claim 1, wherein A is selected from the group consisting of: 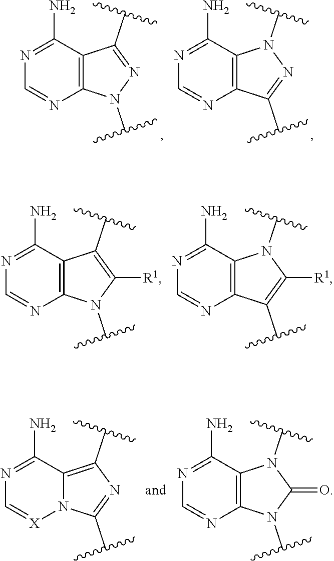
wherein
R¹ is selected from hydrogen, halogen, substituted or unsubstituted alkyl, substituted or unsubstituted heteroalkyl, substituted or unsubstituted cycloalkyl, substituted or unsubstituted heterocyclyl, substituted or unsubstituted aryl, substituted or unsubstituted heteroaryl, substituted or unsubstituted aralkyl, or substituted or unsubstituted heteroaralkyl; and
X is N or CH.

5. The compound according to claim 4, wherein R¹ is hydrogen.

6. The compound according to claim 1, wherein R is methyl.

7. The compound according to claim 1, wherein X¹ is fluorine and m′=1.

8. The compound according to claim 1, wherein X² is hydrogen.

9. The compound according to claim 1, wherein L is selected from: 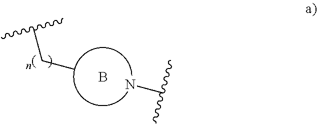
wherein
B is substituted or unsubstituted 3- to 8-membered nitrogen containing heterocyclic ring; and
n is an integer from 0 to 1;
or 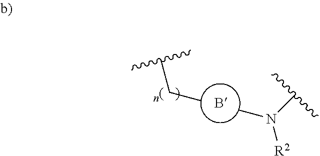
wherein
B′ is substituted or unsubstituted 3- to 8-membered cycloalkyl ring;
n is an integer from 0 to 1; and
R² is selected from hydrogen and lower alkyl.

10. The compound according to claim 1, wherein L-E is selected from the group consisting of:

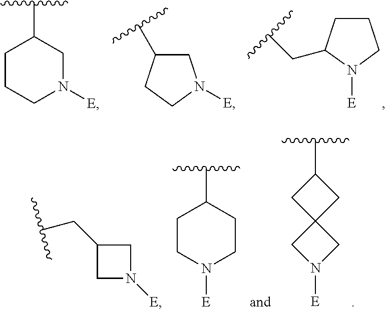

11. The compound according to claim 10, wherein L-E is selected from the group consisting of:

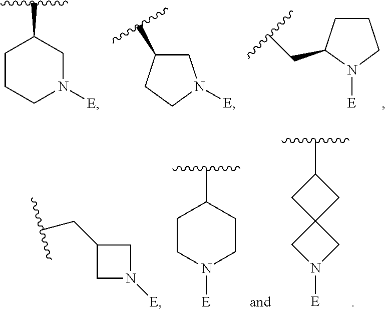

12. The compound according to claim 1, wherein L-E is

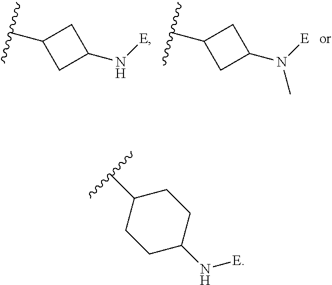

13. The compound according to claim 12, wherein L-E is

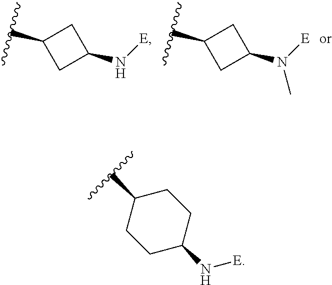

14. The compound according to any one of claims 1 to 13, wherein E is —CN.

15. The compound according to any one of claims 1 to 13, wherein E is selected from the group consisting of:

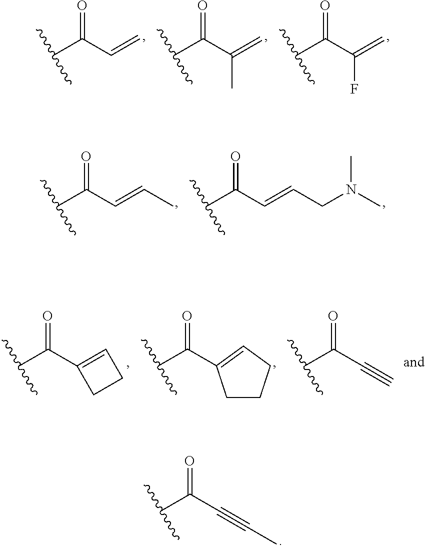

16. The compound according to any one of claims 1 to 13, wherein E is

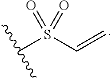 [SMILES: *S(=O)(=O)C=C]

17. The compound of claim 1 wherein Formula I is

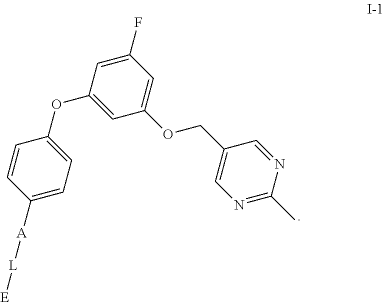

18. The compound of claim 17 wherein A is

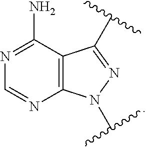 [SMILES: *c1nn(*)c2ncnc(N)c12]

19. A compound of Formula II selected from the group consisting of 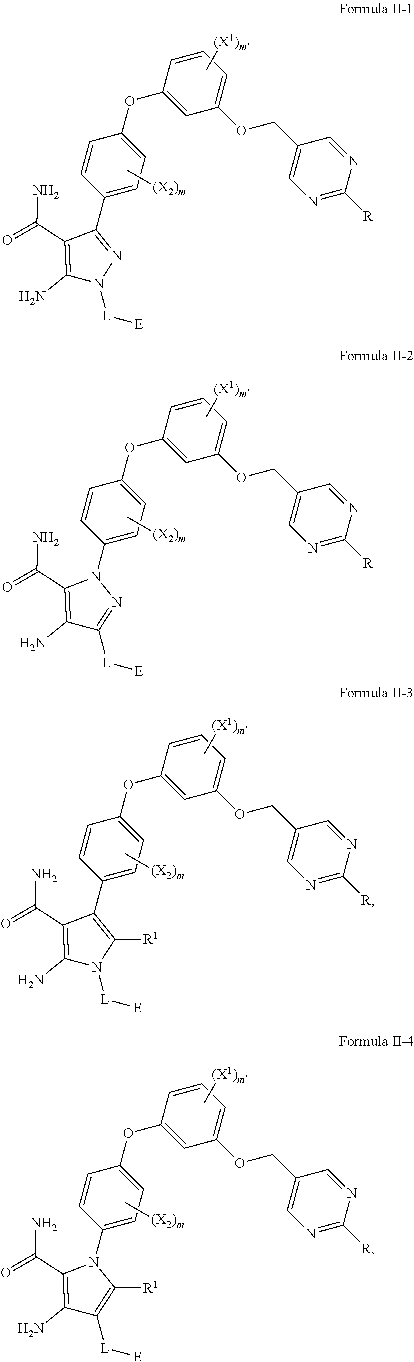 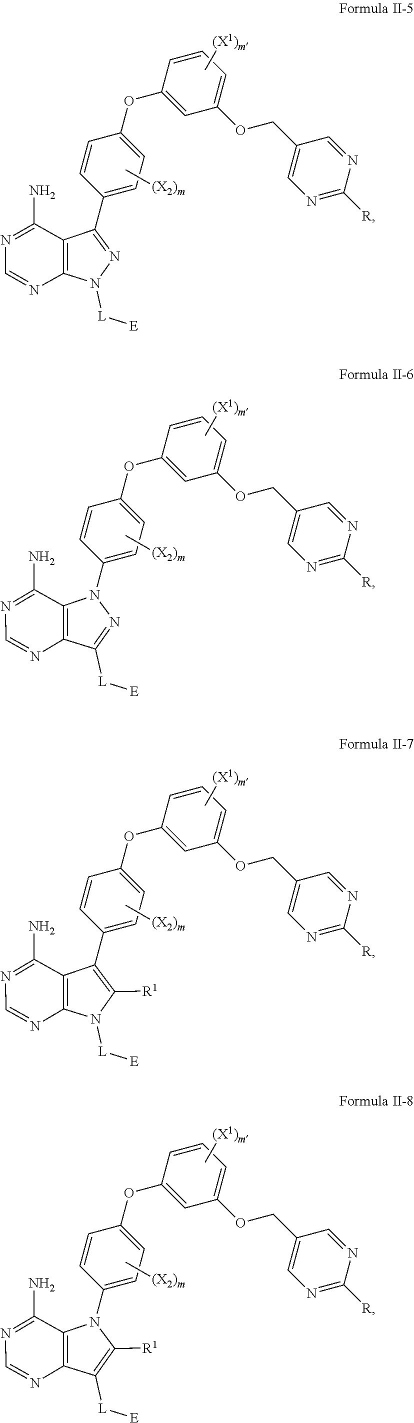 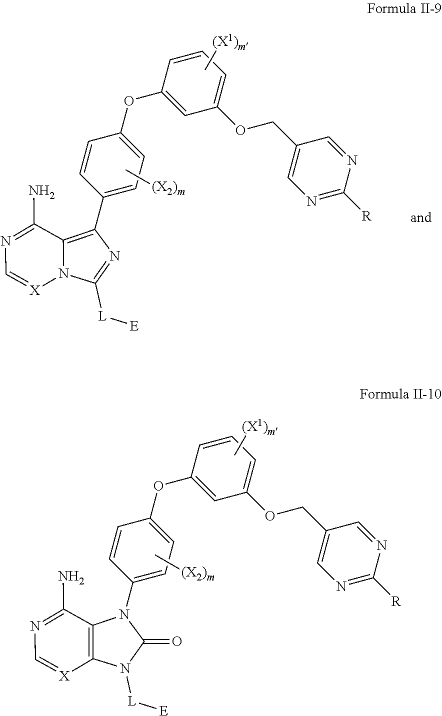
or a pharmaceutically acceptable salt, solvate, solvate of salt, stereoisomer, tautomer, isotope, prodrug, complex or biologically active metabolite thereof, wherein
X¹ and X² are independently selected from hydrogen and halogen;
m is an integer from 0 to 4;
m′ is an integer from 0 to 4;
R is selected from hydrogen and methyl;
L is selected from the group consisting of substituted or unsubstituted alkyl, substituted or unsubstituted heteroalkyl, substituted or unsubstituted cycloalkyl, substituted or unsubstituted heterocyclyl, substituted or unsubstituted aryl, substituted or unsubstituted heteroaryl, substituted or unsubstituted aralkyl, substituted or unsubstituted heteroaralkyl, 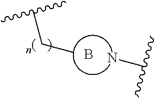
wherein
B is substituted or unsubstituted 3- to 8-membered nitrogen containing heterocyclic ring; and
n is an integer from 0 to 1;
or 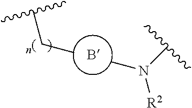
wherein
B′ is substituted or unsubstituted 3- to 8-membered cycloalkyl ring;
n is an integer from 0 to 1; and
R² is selected from hydrogen and lower alkyl;
E is selected from the group consisting of: 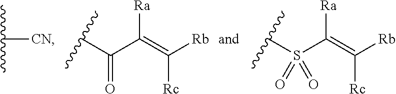
wherein
Ra, Rb and Rc are independently selected from hydrogen, halogen, —CN, substituted or unsubstituted alkyl, substituted or unsubstituted heteroalkyl, substituted or unsubstituted cycloalkyl, or substituted or unsubstituted heterocyclyl; or
Ra and Rb taken together with the carbon atoms to which they are attached form a 3- to 8-membered substituted or unsubstituted cycloalkyl ring, or form a 3-to 8-membered substituted or unsubstituted heterocyclic ring, and Rc is selected as above; or
Rb and Rc taken together with the carbon atom to which they are attached form a 3- to 8-membered substituted or unsubstituted cycloalkyl ring, or form a 3- to 8-membered heterocyclic ring, and Ra is selected as above; or
Ra and Rb taken together with the carbon atoms to which they are attached form a triple bond, and Rc is selected as above.

20. The compound according to claim 19, wherein R is methyl.

21. The compound according to claim 19, wherein X¹ is fluorine and m′=1.

22. The compound according to claim 19, wherein X² is hydrogen.

23. The compound according to claim 19, wherein L is: 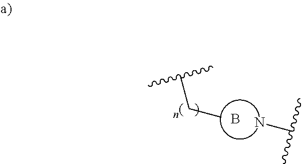
wherein
B is substituted or unsubstituted 3- to 8-membered nitrogen containing heterocyclic ring; and
n is an integer from 0 to 1; or 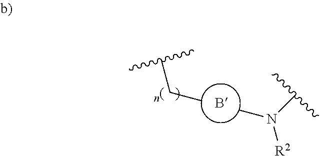
wherein
B′ is substituted or unsubstituted 3- to 8-membered cycloalkyl ring;
n is an integer from 0 to 1; and
R² is selected from hydrogen or methyl.

24. The compound according to claim 19, wherein L-E is selected the group consisting of:

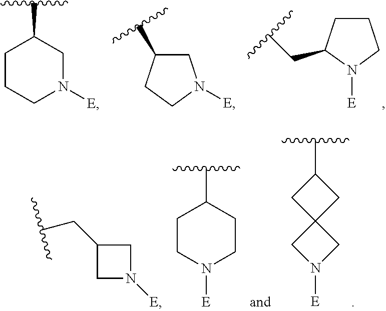

25. The compound according to claim 19, wherein L-E is:

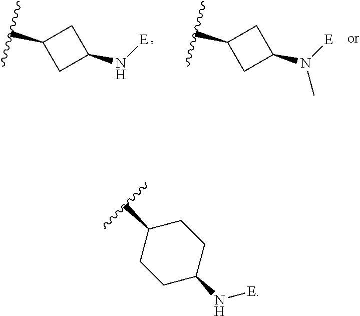

26. The compound according to claim 19, wherein E is —CN.

27. The compound according to claim 19, wherein E is:

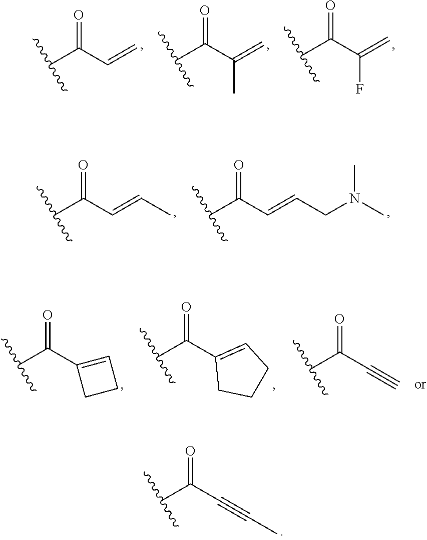

28. The compound according to claim 19, wherein E is

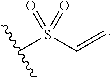 [SMILES: *S(=O)(=O)C=C]

29. The compound according to claim 19, wherein L-E is:

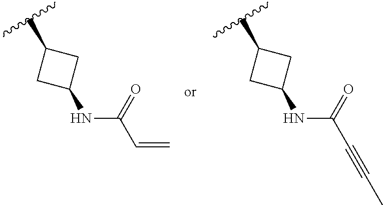

30. A compound of Formula II-5 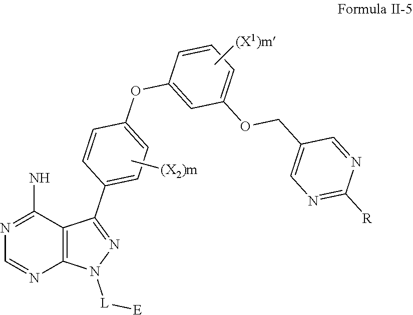
or a pharmaceutically acceptable salt, solvate, solvate of salt, stereoisomer, tautomer, isotope, prodrug, complex or biologically active metabolite thereof, wherein
R is hydrogen or methyl
X¹ and X² are independently selected from hydrogen and halogen;
m is an integer from 0 to 4;
m′ is an integer from 0 to 4;
L is selected from the group consisting of substituted or unsubstituted alkyl, substituted or unsubstituted heteroalkyl, substituted or unsubstituted cycloalkyl, substituted or unsubstituted heterocyclyl, substituted or unsubstituted aryl, substituted or unsubstituted heteroaryl, substituted or unsubstituted aralkyl, substituted or unsubstituted heteroaralkyl, 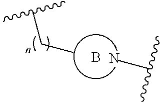
wherein
B is substituted or unsubstituted 3- to 8-membered nitrogen containing heterocyclic ring; and
n is an integer from 0 to 1;
or
wherein 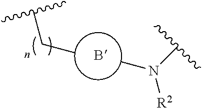
B′ is substituted or unsubstituted 3- to 8-membered cycloalkyl ring;
n is an integer from 0 to 1; and
R² is selected from hydrogen and lower alkyl;
E is selected from the group consisting of: 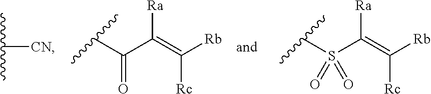
wherein
Ra, Rb and Rc are independently selected from hydrogen, halogen, —CN, substituted or unsubstituted alkyl, substituted or unsubstituted heteroalkyl, substituted or unsubstituted cycloalkyl, or substituted or unsubstituted heterocyclyl; or
Ra and Rb taken together with the carbon atoms to which they are attached form a 3- to 8-membered substituted or unsubstituted cycloalkyl ring, or form a 3-to 8-membered substituted or unsubstituted heterocyclic ring, and Rc is selected as above; or
Rb and Rc taken together with the carbon atom to which they are attached form a 3- to 8-membered substituted or unsubstituted cycloalkyl ring, or form a 3- to 8-membered heterocyclic ring, and Ra is selected as above; or
Ra and Rb taken together with the carbon atoms to which they are attached form a triple bond and Rc is selected as above.

31. The compound according to claim 30, wherein L-E is:

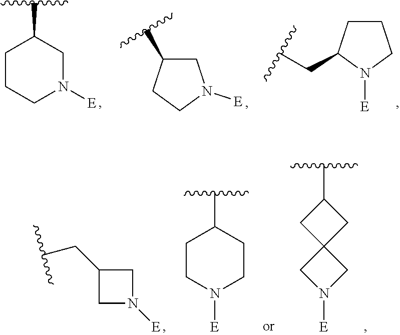

32. The compound according to claim 30, wherein L-E is:

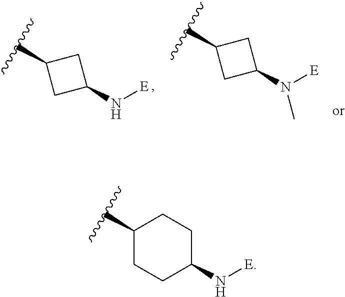

33. The compound according to claim 30, wherein E is —CN.

34. The compound according to claim 30, wherein E is:

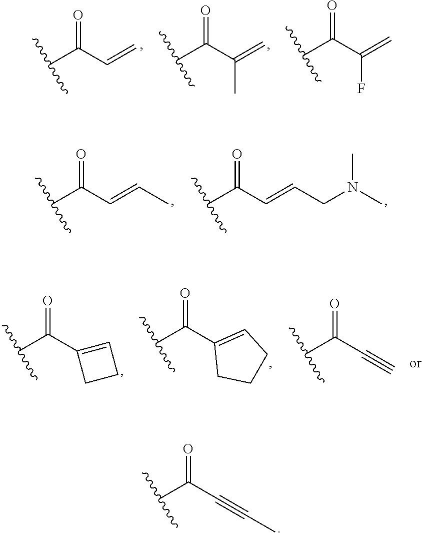

35. The compound according to claim 30, wherein E is

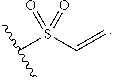 [SMILES: *S(=O)(=O)C=C]

36. The compound according to claim 30 wherein L-E is:

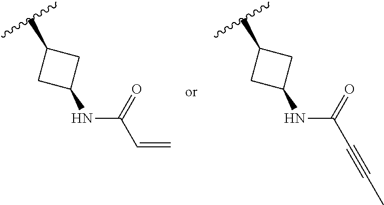

37. The compound according to claim 30, wherein R is methyl.

38. The compound according to claim 30, wherein X¹ is fluorine and m′=1.

39. The compound according to claim 30, wherein X² is hydrogen.

40. A compound selected from the group consisting of:

| Com- | |
|---|---|
| pound | Structure |
| 1 | 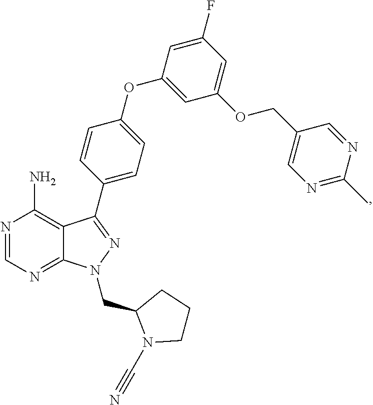 [SMILES: Cc1ncc(COc2cc(F)cc(Oc3ccc(-c4nn(C[C@H]5CCCN5C#N)c5ncnc(N)c45)cc3)c2)cn1] |
| 2 | 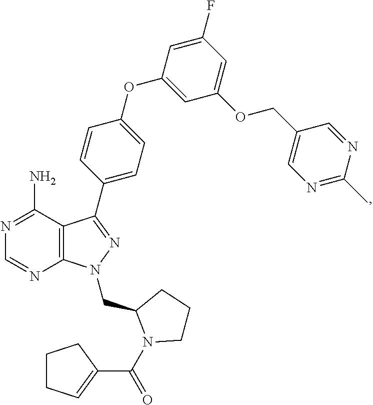 [SMILES: Cc1ncc(COc2cc(F)cc(Oc3ccc(-c4nn(C[C@H]5CCCN5C(=O)C5=CCCC5)c5ncnc(N)c45)cc3)c2)cn1] |
| 3 | 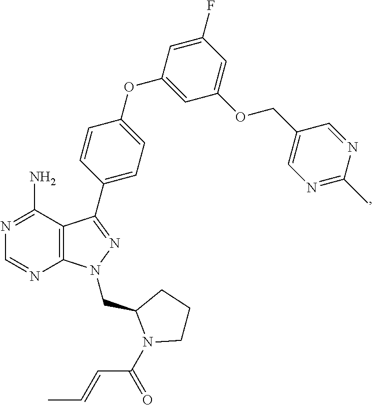 [SMILES: C/C=C/C(=O)N1CCC[C@@H]1Cn1nc(-c2ccc(Oc3cc(F)cc(OCc4cnc(C)nc4)c3)cc2)c2c(N)ncnc21] |
| 4 | 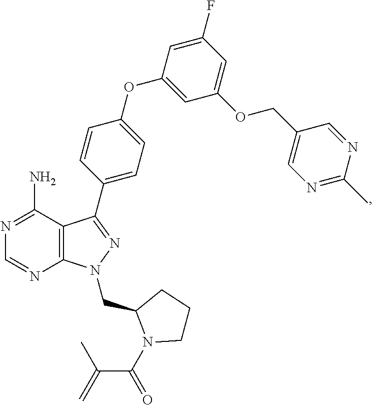 [SMILES: C=C(C)C(=O)N1CCC[C@@H]1Cn1nc(-c2ccc(Oc3cc(F)cc(OCc4cnc(C)nc4)c3)cc2)c2c(N)ncnc21] |
| 5 | 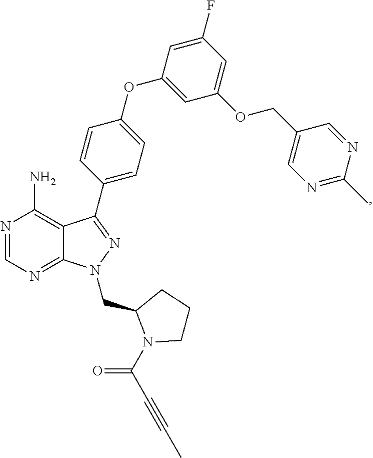 [SMILES: CC#CC(=O)N1CCC[C@@H]1Cn1nc(-c2ccc(Oc3cc(F)cc(OCc4cnc(C)nc4)c3)cc2)c2c(N)ncnc21] |
| 6 | 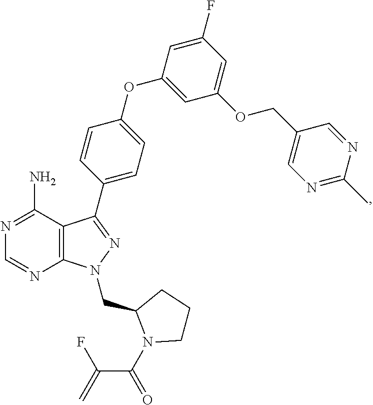 [SMILES: C=C(F)C(=O)N1CCC[C@@H]1Cn1nc(-c2ccc(Oc3cc(F)cc(OCc4cnc(C)nc4)c3)cc2)c2c(N)ncnc21] |
| 7 | 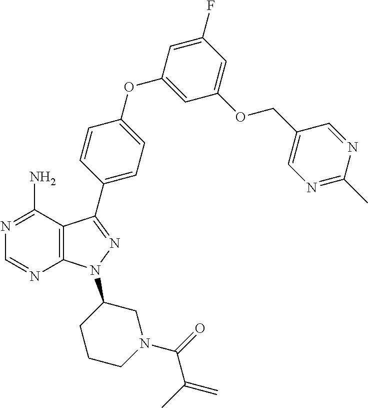 [SMILES: C=C(C)C(=O)N1CCC[C@@H](n2nc(-c3ccc(Oc4cc(F)cc(OCc5cnc(C)nc5)c4)cc3)c3c(N)ncnc32)C1] |
| 8 | 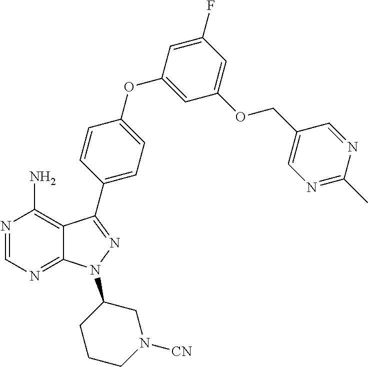 [SMILES: Cc1ncc(COc2cc(F)cc(Oc3ccc(-c4nn([C@@H]5CCCN(C#N)C5)c5ncnc(N)c45)cc3)c2)cn1] |
| 9 | 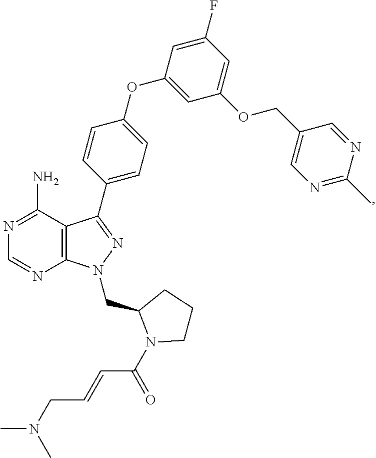 |
| 10 | 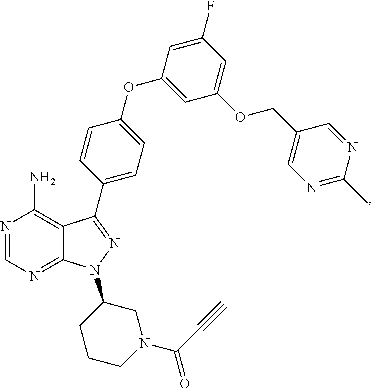 [SMILES: C#CC(=O)N1CCC[C@@H](n2nc(-c3ccc(Oc4cc(F)cc(OCc5cnc(C)nc5)c4)cc3)c3c(N)ncnc32)C1] |
| 11 | 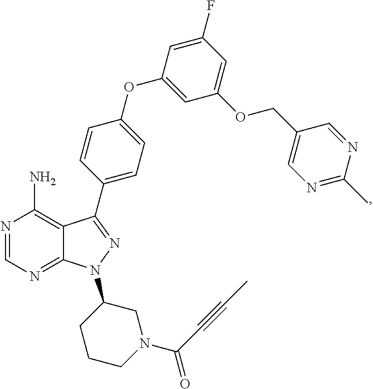 [SMILES: CC#CC(=O)N1CCC[C@@H](n2nc(-c3ccc(Oc4cc(F)cc(OCc5cnc(C)nc5)c4)cc3)c3c(N)ncnc32)C1] |
| 12 | 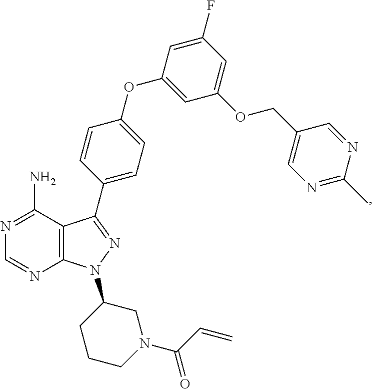 [SMILES: C=CC(=O)N1CCC[C@@H](n2nc(-c3ccc(Oc4cc(F)cc(OCc5cnc(C)nc5)c4)cc3)c3c(N)ncnc32)C1] |
| 13 | 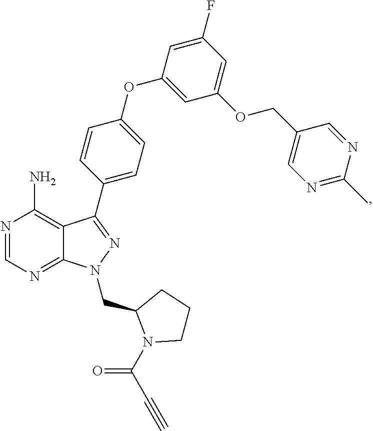 [SMILES: C#CC(=O)N1CCC[C@@H]1Cn1nc(-c2ccc(Oc3cc(F)cc(OCc4cnc(C)nc4)c3)cc2)c2c(N)ncnc21] |
| 14 | 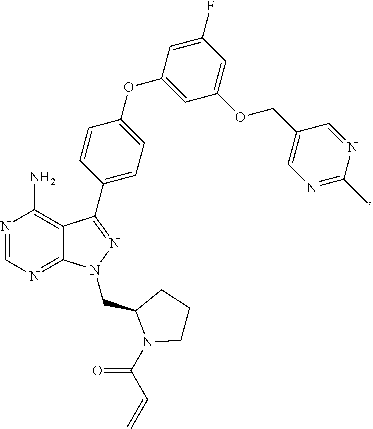 [SMILES: C=CC(=O)N1CCC[C@@H]1Cn1nc(-c2ccc(Oc3cc(F)cc(OCc4cnc(C)nc4)c3)cc2)c2c(N)ncnc21] |
| 15 | 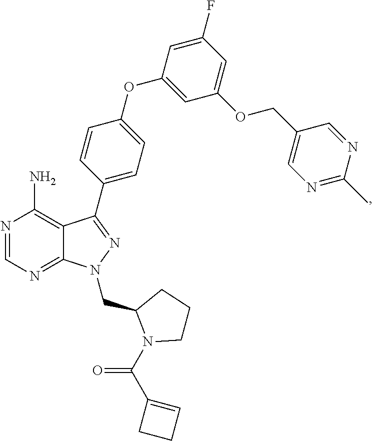 [SMILES: Cc1ncc(COc2cc(F)cc(Oc3ccc(-c4nn(C[C@H]5CCCN5C(=O)C5=CCC5)c5ncnc(N)c45)cc3)c2)cn1] |
| 16 | 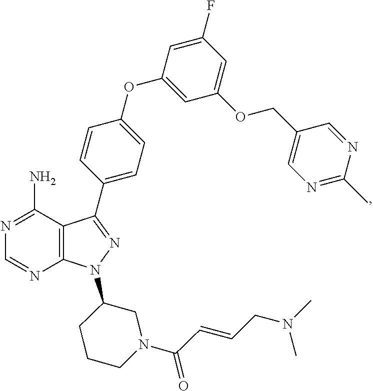 [SMILES: Cc1ncc(COc2cc(F)cc(Oc3ccc(-c4nn([C@@H]5CCCN(C(=O)/C=C/CN(C)C)C5)c5ncnc(N)c45)cc3)c2)cn1] |
| 17 | 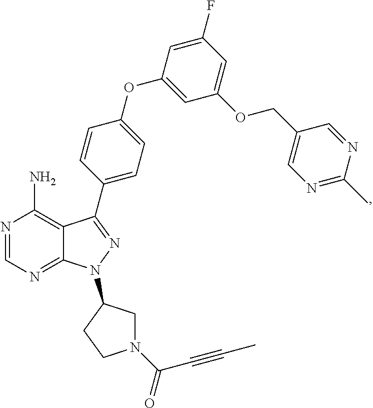 [SMILES: CC#CC(=O)N1CC[C@@H](n2nc(-c3ccc(Oc4cc(F)cc(OCc5cnc(C)nc5)c4)cc3)c3c(N)ncnc32)C1] |
| 18 | 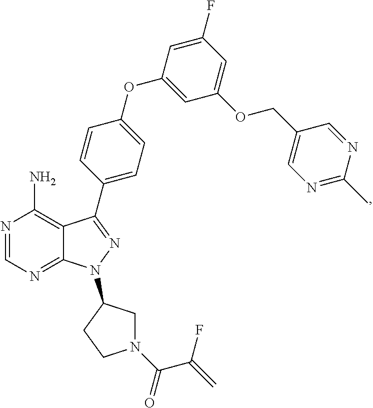 [SMILES: C=C(F)C(=O)N1CC[C@@H](n2nc(-c3ccc(Oc4cc(F)cc(OCc5cnc(C)nc5)c4)cc3)c3c(N)ncnc32)C1] |
| 19 | 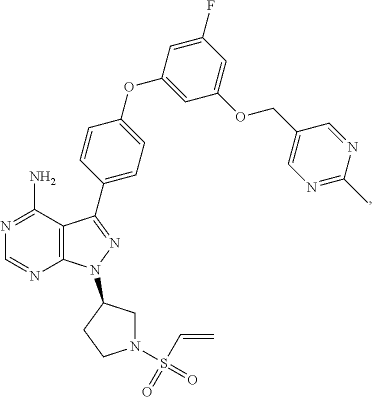 [SMILES: C=CS(=O)(=O)N1CC[C@@H](n2nc(-c3ccc(Oc4cc(F)cc(OCc5cnc(C)nc5)c4)cc3)c3c(N)ncnc32)C1] |
| 20 | 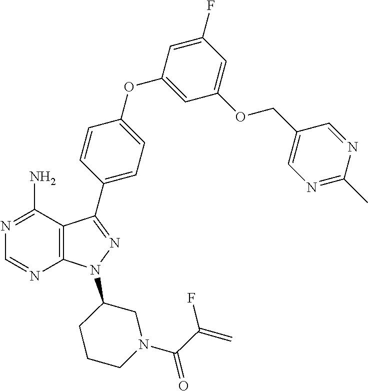 [SMILES: C=C(F)C(=O)N1CCC[C@@H](n2nc(-c3ccc(Oc4cc(F)cc(OCc5cnc(C)nc5)c4)cc3)c3c(N)ncnc32)C1] |
| 21 | 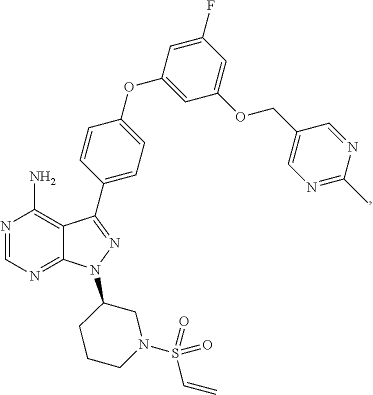 [SMILES: C=CS(=O)(=O)N1CCC[C@@H](n2nc(-c3ccc(Oc4cc(F)cc(OCc5cnc(C)nc5)c4)cc3)c3c(N)ncnc32)C1] |
| 22 | 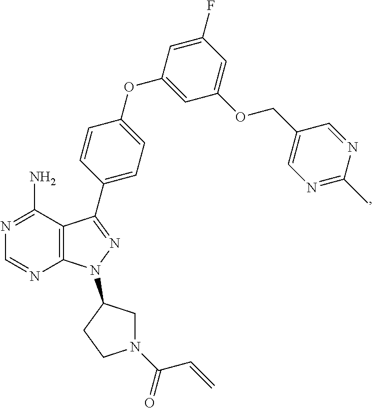 [SMILES: C=CC(=O)N1CC[C@@H](n2nc(-c3ccc(Oc4cc(F)cc(OCc5cnc(C)nc5)c4)cc3)c3c(N)ncnc32)C1] |
| 23 | 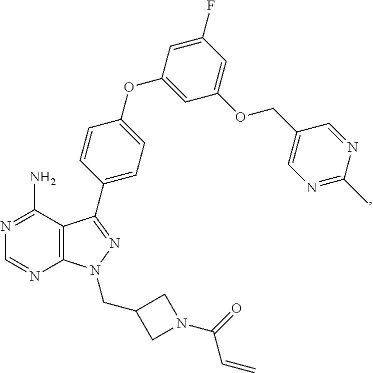 [SMILES: C=CC(=O)N1CC(Cn2nc(-c3ccc(Oc4cc(F)cc(OCc5cnc(C)nc5)c4)cc3)c3c(N)ncnc32)C1] |
| 24 | 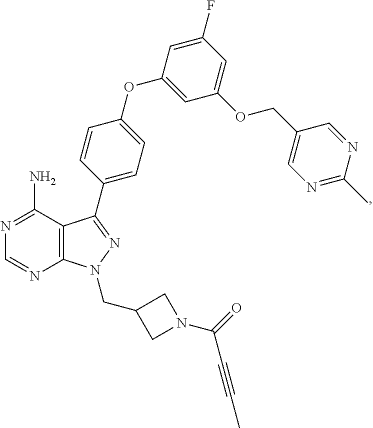 [SMILES: CC#CC(=O)N1CC(Cn2nc(-c3ccc(Oc4cc(F)cc(OCc5cnc(C)nc5)c4)cc3)c3c(N)ncnc32)C1] |
| 25 | 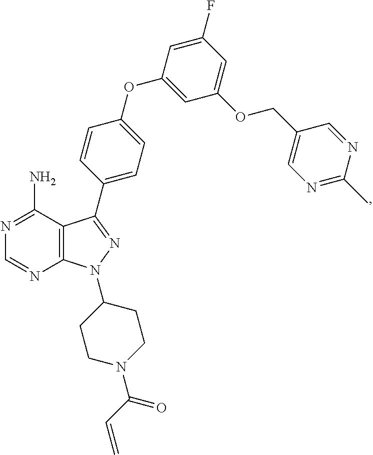 [SMILES: C=CC(=O)N1CCC(n2nc(-c3ccc(Oc4cc(F)cc(OCc5cnc(C)nc5)c4)cc3)c3c(N)ncnc32)CC1] |
| 26 | 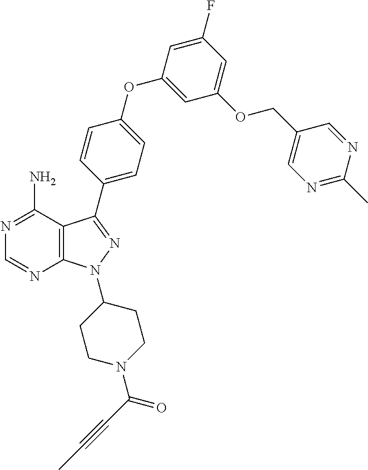 [SMILES: CC#CC(=O)N1CCC(n2nc(-c3ccc(Oc4cc(F)cc(OCc5cnc(C)nc5)c4)cc3)c3c(N)ncnc32)CC1] |
| 27 | 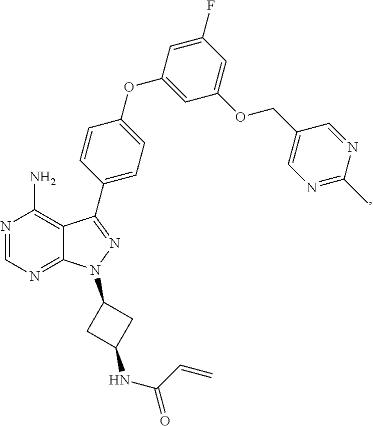 [SMILES: C=CC(=O)N[C@H]1C[C@@H](n2nc(-c3ccc(Oc4cc(F)cc(OCc5cnc(C)nc5)c4)cc3)c3c(N)ncnc32)C1] |
| 28 | 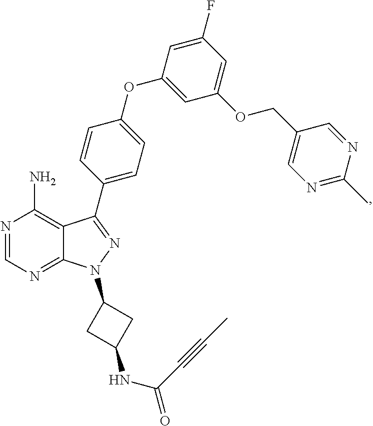 [SMILES: CC#CC(=O)N[C@H]1C[C@@H](n2nc(-c3ccc(Oc4cc(F)cc(OCc5cnc(C)nc5)c4)cc3)c3c(N)ncnc32)C1] |
| 29 | 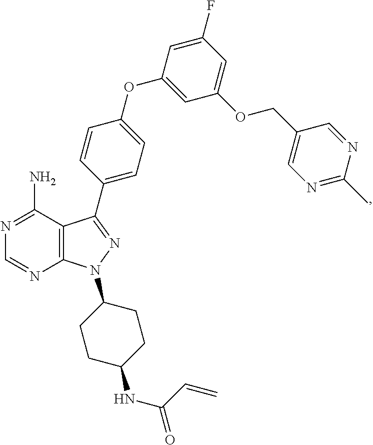 [SMILES: C=CC(=O)N[C@H]1CC[C@@H](n2nc(-c3ccc(Oc4cc(F)cc(OCc5cnc(C)nc5)c4)cc3)c3c(N)ncnc32)CC1] |
| 30 | 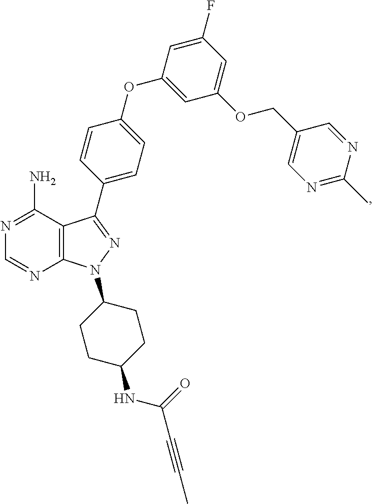 [SMILES: CC#CC(=O)N[C@H]1CC[C@@H](n2nc(-c3ccc(Oc4cc(F)cc(OCc5cnc(C)nc5)c4)cc3)c3c(N)ncnc32)CC1] |
| 31 | 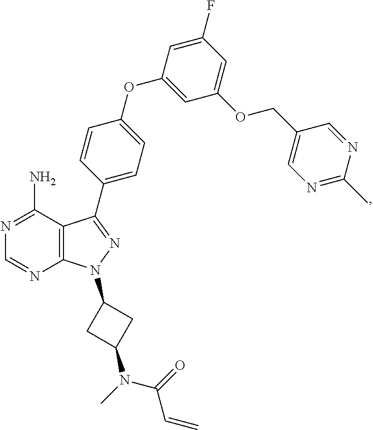 [SMILES: C=CC(=O)N(C)[C@H]1C[C@@H](n2nc(-c3ccc(Oc4cc(F)cc(OCc5cnc(C)nc5)c4)cc3)c3c(N)ncnc32)C1] |
| 32 | 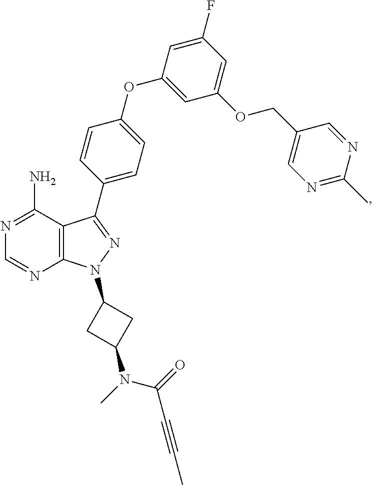 [SMILES: CC#CC(=O)N(C)[C@H]1C[C@@H](n2nc(-c3ccc(Oc4cc(F)cc(OCc5cnc(C)nc5)c4)cc3)c3c(N)ncnc32)C1] |
| 33 | 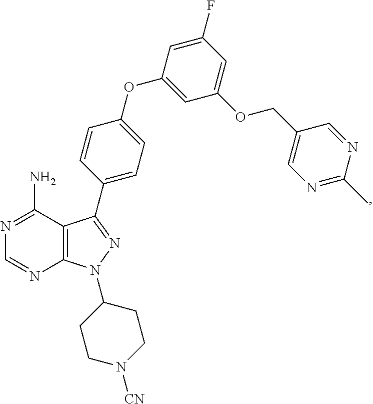 [SMILES: Cc1ncc(COc2cc(F)cc(Oc3ccc(-c4nn(C5CCN(C#N)CC5)c5ncnc(N)c45)cc3)c2)cn1] |
| 34 | 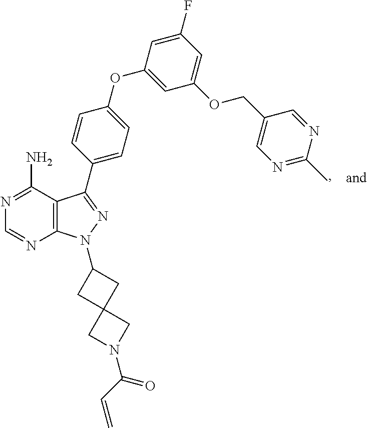 [SMILES: C=CC(=O)N1CC2(CC(n3nc(-c4ccc(Oc5cc(F)cc(OCc6cnc(C)nc6)c5)cc4)c4c(N)ncnc43)C2)C1] |
| 35 | 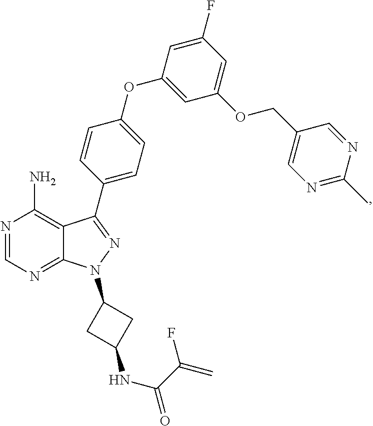 [SMILES: C=C(F)C(=O)N[C@H]1C[C@@H](n2nc(-c3ccc(Oc4cc(F)cc(OCc5cnc(C)nc5)c4)cc3)c3c(N)ncnc32)C1] |

or a pharmaceutically acceptable salt thereof.

41. A pharmaceutical composition comprising the compound of any one of claims 1 to 40 and at least one pharmaceutically acceptable carrier, excipient or diluent.

42. The pharmaceutical composition of claim 41, for use in prevention or treatment of cancer, autoimmune diseases, allergic diseases, inflammatory diseases, graft-versus-host disease, thromboembolic diseases, neurological disorders, viral infections, bone-related diseases or combinations thereof.

43. The compound of any one of claims 1 to 40 for use in therapy, wherein a subject is suffering of a disease, disorder or condition in which one or more Tec kinase family member, or BTK kinase activity is implicated.

44. The pharmaceutical composition according to claim 42 further comprising at least one additional active pharmaceutical ingredient for the treatment or prevention of cancer, autoimmune diseases, allergic diseases, inflammatory diseases, neurological disorders or viral infection in combination therapy.

45. The pharmaceutical composition according to claim 44, wherein the additional active pharmaceutical ingredient is selected from the group consisting of: steroids, leukotriene antagonists, anti-histamines, anti-cancer, anti-viral, anti-biotic agents, protein kinase inhibitors, immune modulators, checkpoint inhibitors or combinations thereof, and wherein additional active pharmaceutical ingredient is administered together with the compounds of Formula I (including Formula I-1) or Formula II (including compounds of Formula II-1 to II-10) or a pharmaceutically acceptable salt or solvate thereof, as a single dosage form, or separately as part of a multiple dosage form.

46. The compound of any one of claims 1 to 40 for use in the manufacture of a medicament or pharmaceutical composition suitable for the prevention or treatment of cancer, autoimmune diseases, allergic diseases, inflammatory diseases, graft-versus-host disease, thromboembolic diseases, neurological disorders, viral infections, bone-related diseases or combinations thereof.

47. A method for treating or preventing a protein kinase mediated disease or condition in a subject, comprising administering to the subject a therapeutically effective amount of a compound of Formula I (including Formula I-1) or Formula II (including compounds of Formula II-1 to II-10), or a pharmaceutically acceptable salt, or solvate thereof.

48. The method according to claim 47, wherein the disease, disorder or condition is associated with TEC family members, and BTK kinase activity.

49. The method according to claim 47 or 48, wherein the compound is used to treat or prevent cancer, autoimmune diseases, allergic diseases, inflammatory diseases, graft-versus-host disease, thromboembolic diseases, neurological disorders, viral infections, bone-related diseases and a combinations thereof.

50. The method of treating according to any one of claims 47 to 49, wherein the enzymatic activity of BTK is reduced by administering to the subject suffering from cancer, autoimmune diseases, allergic diseases, inflammatory diseases, viral infection or combinations thereof, a therapeutically effective amount of the compound of any one of claims 1 to 41, or a pharmaceutically acceptable salt, solvate, solvate of a salt, stereoisomer, tautomer, isotope, prodrug, complex or biologically active metabolite thereof.

51. A method of modulating kinase activity in a subject comprising administering a therapeutically effective amount of the compound of any one of claims 1 to 40, to said subject to modulate the enzymatic activity of a protein kinase.

52. A method of inhibiting protein kinase in a cell or tissue comprising contacting the cell or tissue with an effective amount of the compound of any one of claims 1 to 40, or a pharmaceutically acceptable salt or solvate thereof.

53. A method of inhibiting protein kinase activity, comprising administering to a human or animal subject an effective amount of the compound of any one of claims 1 to 40, or a pharmaceutically acceptable salt or solvate thereof.

54. The method according to claim 50 further comprising administering a therapeutically effective amount of at least one additional active pharmaceutical ingredient for the treatment of cancer, autoimmune diseases, allergic diseases, inflammatory diseases or viral infection in combination therapy, wherein additional active pharmaceutical ingredient is administered together with the compounds of Formula I (including Formula I-1) or Formula II (including compounds of Formula II-1 to II-10) or a pharmaceutically acceptable salt or solvate thereof, as a single dosage form or separately as part of a multiple dosage form.

55. The method according to claim 54, wherein the additional active pharmaceutical ingredient is selected from the group comprising steroids, leukotriene antagonists, anti-histamines, anti-cancer, anti-viral, anti-biotic agents, protein kinase inhibitors, immune modulators, checkpoint inhibitors and a combinations thereof.

56. A probe comprising the compound of any one of claims 1 to 40 covalently conjugated to a detectable label or affinity tag, wherein the detectable label is selected from the group consisting of: a fluorescent moiety, a chemiluminescent moiety, a paramagnetic contrast agent, a metal chelate, a radioactive isotope-containing moiety and biotin.

57. A process for preparing intermediate D1 comprising reacting intermediates of formula C2 and B6 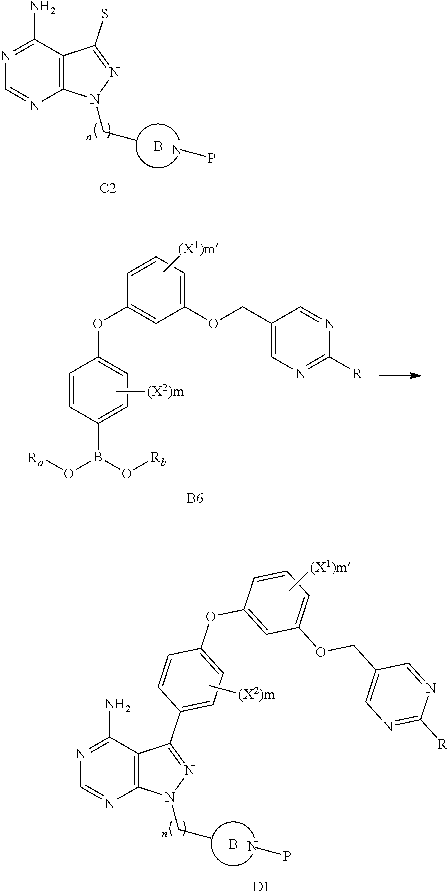
wherein
X is Br or I;
Ra and Rb are independently H, C₁-C₆ alkyl; or
Ra and Rb combine to form a cyclic boronic ester; and
a palladium catalyst mediated coupling conditions to provide Intermediate D1.

58. A process for preparing intermediate G1 comprising reacting intermediates of formula F2 and B6 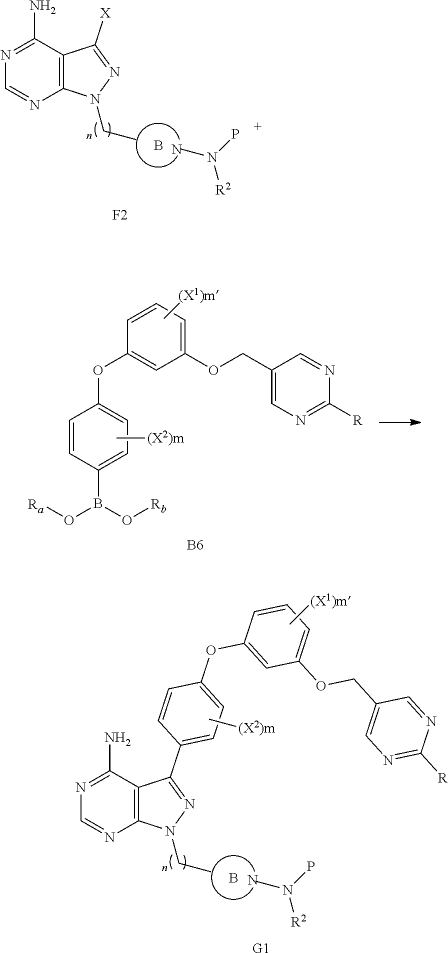
wherein
X is Br or I;
Ra and Rb are independently selected from H, C₁-C₆ alkyl; or
Ra and Rb combine to form a cyclic boronic ester; and
a palladium catalyst mediated coupling conditions to provide Intermediate G1.

59. Use of the compounds of any one of claims 1 to 40 for the treatment of a subject for the prevention or treatment of cancer, autoimmune diseases, allergic diseases, inflammatory diseases, graft-versus-host disease, thromboembolic diseases, neurological disorders, viral infections, bone-related diseases or combinations thereof.

60. The use according to claim 59 wherein the cancer is selected from: B-cell malignancy, B-cell lymphoma, diffuse large B cell lymphoma, chronic lymphocyte leukemia, non-Hodgkin lymphoma for example ABC-DLBCL, mantle cell lymphoma, follicular lymphoma, hairy cell leukemia B-cell non-Hodgkin lymphoma, Waldenstrom's macroglobulinemia, multiple myeloma, bone cancer, bone metastasis, or solid tumors.

61. The use according to claim 59 wherein the autoimmune disease is selected from: rheumatoid arthritis, juvenile rheumatoid arthritis, osteoarthritis, ankylosing spondylitis, psoriatic arthritis, psoriasis vulgaris, pemphigus vulgaris, bullous pemphigoid, Sjogren's syndrome, systemic lupus erythromatosus, discoid SLE, lupus nephritis, antiphospholipidosis, whipple, dermatomyositis, polymyositis, autoimmune thrombocytopenia, idiopathic thrombocytopenia purpura, thrombotic thrombocytopeni a purpura, autoimmune (cold) agglutinin disease, autoimmune hemolytic anemia, cryoglobulinemia, autoimmune vasculitis, ANCA-associated vasculitis, scleroderma, systemic sclerosis, multiple sclerosis, chronic focal encephalitis, Guillian-Barre syndrome, chronic fatigue syndrome, mononucleosis, neuromyelitis optica, autoimmune uveitis, Grave's disease, thyroid associated opthalmopathy, granulomatosis with microscopic polyangitis, Wegeners granulomatosis, idiopathic pulmonary fibrosis, sarcoidosis, idiopathic membranous nephropathy, IgA nephropathy, glomerulos clerosis, pancreatitis, type I diabetes or type II diabetes.

62. The use according to any one of claims 59 to 61 further comprising the co-administration of a therapeutically effective amount of at least one additional active pharmaceutical ingredient for the treatment of cancer, autoimmune diseases selected from: rheumatoid arthritis, juvenile rheumatoid arthritis, osteoarthritis, ankylosing spondylitis, psoriatic arthritis, psoriasis vulgaris, pemphigus vulgaris, bullous pemphigoid, Sjogren's syndrome, systemic lupus erythromatosus, discoid SLE, lupus nephritis, antiphospholipidosis, whipple, dermatomyositis, polymyositis, autoimmune thrombocytopenia, idiopathic thrombocytopenia purpura, thrombotic thrombocytopenia purpura, autoimmune (cold) agglutinin disease, autoimmune hemolytic anemia, cryoglobulinemia, autoimmune vasculitis, ANCA-associated vasculitis, scleroderma, systemic sclerosis, multiple sclerosis, chronic focal encephalitis, Guillian-Barre syndrome, chronic fatigue syndrome, mononucleosis, neuromyelitis optica, autoimmune uveitis, Grave's disease, thyroid associated opthalmopathy, granulomatosis with microscopic polyangitis, Wegeners granulomatosis, idiopathic pulmonary fibrosis, sarcoidosis, idiopathic membranous nephropathy, IgA nephropathy, glomerulos clerosis, pancreatitis, type I diabetes or type II diabetes, allergic diseases, inflammatory diseases, neurological disorders or viral infection in combination therapy, wherein additional active pharmaceutical ingredient is administered together with the compounds of Formula I (including Formula I-1) or Formula II (including compounds of Formula II-1 to II-10) or a pharmaceutically acceptable salt or solvate thereof, as a single dosage form or separately as part of a multiple dosage form.
